(19) United States
(12) Patent Application Publication
(10) Pub. No.: US 2022/0195059 A1
(43) Pub. Date: Jun. 23, 2022

(54) Rank Pathway Inhibitors in Combination with CDK Inhibitors

(71) Applicant: INSTITUTO DE MEDICINA MOLECULAR JOÃO LOBO ANTUNES, Lisboa (PT)

(72) Inventors: Luis António Marques Da Costa, Lisboa (PT); Sandra Cristina Cara De Anjo Casimiro, Barreiro (PT)

(21) Appl. No.: 17/606,103

(22) PCT Filed: Apr. 24, 2020
(86) PCT No.: PCT/PT20/50017

§ 371 Date: Oct. 25, 2021

Related U.S. Application Data
(60) Provisional application No. 62/840,810, filed on Apr. 30, 2019.

(30) Foreign Application Priority Data
Apr. 30, 2019 (PT) 115483

(51) Int. Cl. C07K 16/28; A61K 31/519; A61K 45/06; A61P 35/00

(52) U.S. Cl. CPC C07K 16/2875; A61K 31/519; A61K 2039/505; A61P 35/00; A61K 45/06

(57) ABSTRACT

Provided herein are pharmaceutical compositions comprising i) a RANK pathway inhibitor in combination with ii) a CDK inhibitor, and related methods. Provided herein are methods of increasing or restoring a responsiveness or sensitivity of a cancer cell to treatment with a CDK inhibitor and methods of treating a subject with a resistance or reduced sensitivity to treatment with a CDK inhibitor.

### CROSS REFERENCE TO RELATED APPLICATIONS

This application claims priority to U.S. Provisional Patent Application No. 62/840,810, filed on Apr. 30, 2019, and Portuguese Patent Application no. 115483, filed on Apr. 30, 2019, the contents of which are incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 130,214 byte ASCII (Text) file named “53978A_Seqlisting.txt”; created on Apr. 22, 2020.

### BACKGROUND

Breast cancer accounts for about 25% of all malignancies worldwide, and in the United States, it is the most common cancer in women (American Cancer Society. Cancer Facts and FIGURES 2019. Atlanta, Ga.: American Cancer Society, 2019; and Ghoncheh et al., Asian Pac J Cancer Prev 17: 43-46 (2016)). It is estimated that, in this year alone, there will be over 265,000 new cases of invasive breast cancer and more than 40,000 deaths caused by this disease (American Cancer Society. Cancer Facts and FIGURES 2019. Atlanta, Ga.: American Cancer Society, 2019).

Based on its estrogen receptor (ER), progesterone receptor (PR), and human epidermal growth factor receptor 2 (HER2) status, breast cancer is categorized into one of three subtypes: hormone receptor (HR)-positive, HER2-positive and triple negative subtypes. Of these subtypes, HR-positive breast cancers account for the majority (Hart et al., Nat Rev Clin Oncol 12: 541-552 (2015)). Hormone therapy (also referred to as “endocrine therapy”) is considered as a mainstay therapy for HR-positive breast cancer, and includes treatment with drugs, such as leuprolide, goserelin, anastrozole, letrozole, exemestane, tamoxifen, toremifene, and fulvestrant. Despite the long success achieved with such hormone therapies, a large number of patients ultimately acquire resistance to hormone therapy (Hoffmann et al., J Natl Cancer Inst 96: 210-218 (2004)) and require other cancer treatments.

The advent of cyclin dependent kinases 4 and 6 (CDK4/6) inhibitors for the treatment of breast cancer patients arose from the increasing number of cancer cases exhibiting resistance to hormone therapy. CDK4/6 inhibitors represented a promising class of drugs as such agents function to interrupt the growth of cancer cells by inhibiting the action of kinases, CDK4 and CDK6, which associate with Cyclin D during transition from G1 to S phase of the cell cycle. Studies with CDK4/6 inhibitors demonstrated an increase in progression-free survival (PFS) of patients treated with CDK4/6 inhibitors, and the therapeutic effectiveness of these treatments is well documented. See, Pandey et al., International Journal of Cancer 2018 (doi.org/10.1002/ijc.32020); and references cited therein. Consequently, the Food and Drug Administration (FDA) approved the use of three CDK4/6 inhibitors, palbociclib, ribociclib and abemaciclib, for the treatment of breast cancer.

Though breast cancer patients were and continue to be successfully treated with CDK4/6 inhibitors, not all patients respond to these drugs and most patients whose tumors respond to CDK4/6 inhibitors eventually develop resistance to these drugs. Pandey et al., 2018, supra. Thus, there is a need in the art for new pharmaceutical compositions able to treat breast cancer patients including those with a resistance to CDK inhibitors, such as CDK4/6 inhibitors.

### SUMMARY OF THE INVENTION

Presented herein for the first time are data demonstrating that Receptor Activator of Nuclear Factor kappa-p (RANK) expression levels are correlated with the sensitivity of cancer cells to CDK inhibitors and that inhibition of the RANK pathway sensitizes such cancer cells to these CDK inhibitors. Accordingly, the present disclosure provides a pharmaceutical composition comprising i) a RANK pathway inhibitor in combination with ii) a CDK inhibitor. In exemplary embodiments, the pharmaceutical composition comprises i) a RANK pathway inhibitor in combination with ii) a CDK4/6 inhibitor.

The present disclosure also provides methods of increasing or restoring responsiveness or sensitivity of a cancer cell to treatment with a CDK inhibitor, optionally, wherein the CDK inhibitor is a CDK4/6 inhibitor. In exemplary embodiments, the method comprises administering to the subject a RANK pathway inhibitor optionally in combination with a CDK inhibitor (e.g., CDK4/6 inhibitor). In exemplary embodiments, the subject is or has been treated with a CDK inhibitor (e.g., CDK4/6 inhibitor) and the method comprises administering a RANK pathway inhibitor to the subject.

Additionally provided herein are methods of treating cancer in a subject. In exemplary embodiments, the method comprises administering to the subject a RANK pathway inhibitor optionally in combination with a CDK inhibitor (e.g., CDK4/6 inhibitor). In exemplary embodiments, the subject is or has been treated with a CDK inhibitor (e.g., CDK4/6 inhibitor) and the method comprises administering to the subject a RANK pathway inhibitor.

Provided herein are methods of treating a subject with cancer. In exemplary aspects, the method comprises administering to the subject a RANK pathway inhibitor optionally in combination with the CDK inhibitor. Optionally, the cancer has a reduced responsiveness to treatment with a CDK inhibitor. In exemplary aspects, (i) cells of the cancer overexpress one or more of RANK, CDK 4, CDK 6, or Cyclin D, (ii) the subject has an increased level of circulating tumor cells (CTCs), or (iii) a combination thereof, and the method comprises administering to the subject a RANK pathway inhibitor optionally in combination with the CDK inhibitor (e.g., CDK4/6 inhibitor).

Provided herein are methods of delaying the occurrence or onset of metastasis in a subject with cancer. In exemplary embodiments, the method comprises administering a RANK pathway inhibitor to the subject, optionally in combination with a CDK inhibitor (e.g., CDK4/6 inhibitor). In exemplary embodiments, the subject is or has been treated with a CDK inhibitor (e.g., CDK4/6 inhibitor) and the method comprises administering to the subject a RANK pathway inhibitor.

Also provided herein are methods of reducing tumor growth or tumor burden or increasing tumor regression in a subject. In exemplary embodiments, the method comprises administering to the subject a RANK pathway inhibitor optionally in combination with a CDK inhibitor (e.g., CDK4/6 inhibitor). In exemplary embodiments, the subject is or has been treated with a CDK inhibitor (e.g., CDK4/6 inhibitor) and the method comprises administering to the subject a RANK pathway inhibitor.

The present disclosure moreover provides a method of increasing progression-free survival (PFS), overall survival (OS), or time to deterioration of Eastern Cooperative Oncology Group (ECOG) performance status in a subject with a cancer. In exemplary embodiments, the method comprises administering to the subject a RANK pathway inhibitor optionally in combination with a CDK inhibitor (e.g., CDK4/6 inhibitor). In exemplary embodiments, the subject is or has been treated with a CDK inhibitor (e.g., CDK4/6 inhibitor) and the method comprises administering a RANK pathway inhibitor to the subject. Optionally, the cancer is resistant to or has a reduced sensitivity to a CDK inhibitor.

Methods of reducing the level of circulating tumor cells (CTCs) in a subject are further provided herein. In exemplary embodiments, the method comprises administering to the subject a RANK pathway inhibitor optionally in combination with a CDK inhibitor (e.g., CDK4/6 inhibitor). In exemplary embodiments, the subject is or has been treated with a CDK inhibitor (e.g., CDK4/6 inhibitor) and the method comprises administering a RANK pathway inhibitor to the subject.

With regard to the foregoing, the cancer in exemplary aspects is resistant to or exhibits a reduced sensitivity to a CDK inhibitor (e.g., CDK4/6 inhibitor). In exemplary embodiments, the RANK pathway inhibitor inhibits a binding interaction between RANK and RANK ligand (RANKL). In exemplary aspects, the RANK pathway inhibitor comprises osteoprotegerin (OPG), a RANKL-binding fragment thereof, or an antigen-binding protein that binds to RANK or RANKL. Optionally, the antigen-binding protein comprises a fully human antibody, a humanized antibody, or a chimeric antibody or a Fab, Fab′, F(ab′)2, or a single chain Fv comprising one, two, three, four, five or more of the heavy and light chain complementarity determining region (CDR) of an anti-RANK antibody or an anti-RANKL antibody. In various aspects, the antigen-binding protein binds to RANKL. In some instances, the antigen-binding protein comprises one, two, three, four, five or more of the heavy and light chain complementarity determining region (CDR) of the antibody called denosumab. In various instances, the antigen-binding protein comprises the amino acid sequences of SEQ ID NOs: 5, 6, 7, 8, 9, and 10. In various aspects, the antigen-binding protein comprises an amino acid sequence of SEQ ID NO: 1 and an amino acid sequence of SEQ ID NO: 2. In various aspects, the antigen-binding protein comprises an amino acid sequence of SEQ ID NO: 1 and an amino acid sequence of SEQ ID NO: 2 and further comprises an amino acid sequence of SEQ ID NO: 16 and an amino acid sequence of SEQ ID NO: 28. In various aspects, the antigen-binding protein comprises an amino acid sequence of SEQ ID NO: 13 and an amino acid sequence of SEQ ID NO: 14. In various aspects, the antigen-binding protein comprises an amino acid sequence of SEQ ID NO: 3 and an amino acid sequence of SEQ ID NO: 4. In exemplary embodiments, the CDK inhibitor (e.g., CDK4/6 inhibitor) is a serine/threonine kinase inhibitor, a Cytochrome P450 (CYP450) 3A Inhibitor, or both. In various aspects, the CDK inhibitor inhibits the phosphorylation of retinoblastoma (Rb) protein. In some aspects, the CDK inhibitor is a CDK4/6 inhibitor. In exemplary aspects, the CDK4/6 inhibitor comprises a structure of Structure I or Structure II:

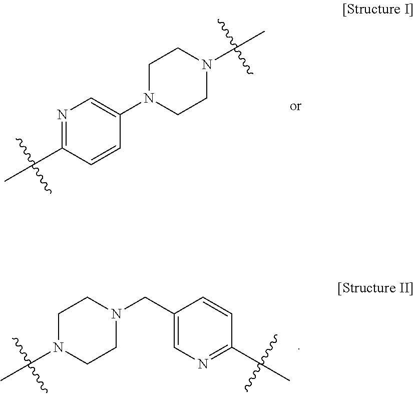 [SMILES: *c1ccc(N2CCN(*)CC2)cn1 ; *c1ccc(CN2CCN(*)CC2)cn1]

In various aspects, the CDK4/6 inhibitor comprises a structure of Structure I or Structure II and further comprises a structure of A-B, wherein A comprises a bicyclic structure and B comprises a monocyclic structure. In exemplary aspects, A-B comprises a structure of Structure III or Structure IV or Structure V:

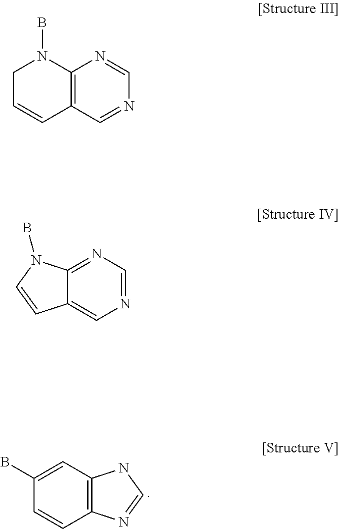

In exemplary aspects, B of Structure III or IV is a cyclopentane. In exemplary aspects, B of Structure V comprises a pyrimidine. In various aspects, the CDK4/6 inhibitor comprises the structure of palbociclib, ribociclib, or abemaciclib, or a pharmaceutically acceptable salt thereof.

In various instances, the pharmaceutical composition comprises additional active ingredients, e.g., a chemotherapeutic agent. Optionally, the pharmaceutical composition comprises an aromatase inhibitor (e.g., letrozole, anastrozole, or exemestante), an ER-targeted agent (e.g., fulvestrant or tamoxifen), rapamycin, a rapamycin analog (e.g., everolimus, temsirolimus, ridaforolimus, zotarolimus, and 32-deoxo-rapamycin), an anti-HER2 drug (e.g., trastuzumab, pertuzumab, lapatinib, T-DM1, or neratinib) or a PI3K inhibitor (e.g., taselisib, alpelisib or buparlisib).

Additional embodiments and aspects of the presently disclosed pharmaceutical compositions and methods are provided below.

### BRIEF DESCRIPTION OF THE DRAWINGS

FIGS. 1A and 1B provide data that show cells transduced by RANK-encoding lentivirus stably overexpressed RANK. FIG. 1A is a graph of relative RANK mRNA expression in Luminal BC cell lines (MCF-7 and T47D) and their derived RANK OE (overexpressing) clones as determined by RT-qPCR. MDA-MB-231 was tested alongside for comparison as a control. Ct values were normalized against the 18S gene. Data is presented as mean±SEM. FIG. 1B is a graph of RANK protein levels as assessed by flow cytometry.

FIG. 1C provides data that show the activation of RANK signaling pathway. After 24 h in low-serum medium, MCF-7 and T47D cells, or their RANK-overexpressing counterparts MCF-7^{OE} and T47D^{OE}, were stimulated with 1 μg/mL soluble RANK ligand (sRANKL) for 0, 5, 10, 20, 40, or 60 min (top panel) or 0, 5, 10, or 25 min (bottom panel). Cell lysates were western blotted using the antibody specific for the indicated protein (IκBα; NK-κB p65; ERK1/2; AKT), or for a phosphorylated version thereof (p-IκBα (Ser32); p-NK-κB p65 (Ser536); p-ERK1/2; p-AKT). β-Actin was used as loading control.

FIGS. 2A and 2B provide data from western blots showing the phenotypic characteristics of RANK OE cells (MCF-7^{OE} and T47O^{OE}) compared to their parental counterparts not overexpressing RANK. FIG. 2A demonstrates decreased protein expression of epithelial marker β-catenin and increased expression of mesenchymal markers like N-cadherin, vimentin, Snail and Slug. FIG. 2B demonstrates increased protein expression of stem cell markers: OCT4, NANOG, and SOX2. β-Actin was used as loading control.

FIG. 2C provides a graph of the % spheres per seeded cells as a measure of Sphere Forming Capacity (SFC) in RANK OE cells (MCF-7^{OE} and T47D^{OE}) compared to their parental counterparts. Adherent cells were cultured in non-adherent conditions and SFC (%) was determined as the number of mammospheres >50 μm/number of cells seeded)×100, after 7 days.

FIG. 3 provides data showing that RANK overexpression decreases sensitivity to fulvestrant. Cells were seeded in 96-well plates and exposed to fulvestrant for seven days. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001. FIG. 3 is a graph of RFU (% from control) as a function of fulvestrant concentration.

FIG. 4 is a series of graphs demonstrating that RANK overexpression decreases sensitivity to CDK4/6 inhibitors (palbociclib, ribociclib and abemaciclib) and further that RANK pathway blockage with osteoprotegerin (OPG-Fc) restored sensitivity to therapy. Cells were seeded in 96-well plates and exposed to different drugs for seven days. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001. The three graphs on the left of FIG. 4 are graphs of MCF-7 cells, MCF-7^{OE} cells, or MCF-7^{OE} cells treated with OPG-Fc that were treated with palbociclib (top), ribociclib (middle), or abemaciclib (bottom). The three graphs on the right of FIG. 4 are graphs of T47D cells, T47D^{OE} cells, or T47D^{OE} cells treated with OPG-Fc that were treated with palbociclib (top), ribociclib (middle), or abemaciclib (bottom).

FIG. 5 is a series of graphs demonstrating that RANK overexpression decreases sensitivity to CDK4/6 inhibitors palbociclib and ribociclib alone or in combination with fulvestrant or everolimus, and further that osteoprotegerin (in this example, full-length OPG, flOPG, was used) restored sensitivity to therapy. Cells were seeded in 96-well plates and exposed to different drugs for seven days. Medium was replaced every two days, and viability was assessed by Alamar blue assay. Results are the mean of three independent assays, with 4 replicates per assay, and presented as the mean±SEM. p-value was calculated using unpaired t-test, *p<0.05, **p<0.01, ***p<0.001. The top four graphs of FIG. 5 are graphs of MCF-7 cells, MCF-7^{OE} cells, or MCF-7^{OE} cells treated with OPG that were treated with either palbociclib and fulvestrant (left-most), palbociclib and everolimus (second from left), ribociclib and fulvestrant (third from left), or ribociclib and everolimus (right-most). The bottom four graphs of FIG. 5 are graphs of T47D cells, T47D^{OE} cells, or T47D^{OE} cells treated with OPG that were treated with either palbociclib and fulvestrant (left-most), palbociclib and everolimus (second from left), ribociclib and fulvestrant (third from left), or ribociclib and everolimus (right-most).

FIGS. 6A and 6B provide data demonstrating that MDA-MB-231 TNBC cells are resistant to CDK4/6 inhibitors palbociclib and ribociclib, and that RANK KD (knockdown) increased sensitivity to therapy. FIG. 6A is a graph of relative RANK mRNA expression in triple negative BC cell line MDA-MB-231 transfected with control or RANK shRNA as determined by RT-qPCR. Ct values were normalized against the 18S gene. FIG. 6B provides data demonstrating that MDA-MB-231 TNBC cells are resistant to CDK4/6 inhibitors palbociclib (left graph) and ribociclib (right graph), and RANK KD increased sensitivity to therapy. Cells were seeded in 96-well plates and exposed to palbociclib (left graph) or ribociclib (right graph) for seven days. Medium was replaced every two days, and viability was assessed by Alamar blue assay. Results are the mean of three independent assays, with 4 replicates per assay, and presented as the mean±SEM. p-value was calculated using 2-way ANOVA, *p<0.05, **p<0.01, ***p<0.001.

FIG. 7 is a series of graphs demonstrating that RANK pathway inhibition with OPG-Fc increases sensitivity of TNBC cell lines to CDK4/6 inhibitors palbociclib, ribociclib and abemaciclib. TNBC cells (MDA-MB-231 (left column of graphs), MDA-MB-468 (middle column of graphs), or MDA-MB-436 (right column of graphs)) were seeded in 96-well plates and exposed to palbociclib (top row), ribociclib (middle row), or abemaciclib (bottom row) for seven days with or without OPG-Fc. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001.

FIGS. 8A and 8B are western blots demonstrating CyclinD1/CDK4/CDK2 up-regulation in response to CDK4/6 inhibitors, where CDK4 up-regulation is abrogated by RANK pathway blockage with OPG-Fc, restoring sensitivity of RANK OE cells. Protein expression was assessed by western blot. Cells (MCF-7 or MCF-7^{0E}) were seeded in 6-well plates and exposed to palbociclib or palbociclib with OPG-Fc for 72 h (FIG. 8A) or with OPG-Fc for seven days (FIG. 8B). β-Actin was used as loading control and normalized levels of Cyclin D1 and CDK4 expression are shown (FIG. 8C).

FIGS. 9A-9C provide data which demonstrate that RANK OE tumors have decreased proliferation rate in vivo. Nod scid gamma (NSG) mice were inoculated in the 2^{nd} thoracic mammary fat pad with MCF-7GFP+Luc+ (Parental), MCF-7 RANK OE GFP+Luc+ (RANK OE) or MCF-7 GFP+Luc+ and MCF-7 RANK OE GFP+Luc+RFP+ cells (1:1) (Mix) (n=5/group). Tumors were imaged by bioluminescence every week post-tumor inoculation tills the end of the experience (twelve weeks). FIG. 9A is a series of representative images of bioluminescence taken at the end of experiment. FIG. 9B is a graph of Total flux (p/s) plotted of time (weeks) post inoculation with the indicated cells. FIG. 9C is a graph of the % Ki67-positive stained cells (Imunoratio) for tumors in mice inoculated with the indicated cells. Data is presented as mean±SEM. p-value was calculated using 2-way ANOVA or unpaired t-test, *p<0.05, **p<0.01, ***p<0.001.

FIGS. 10A-10C demonstrate that exogenous sRANKL does not affect MCF-7^{OE} xenograft growth. Nod scid gamma (NSG) mice were inoculated in the 2nd thoracic mammary fat pad with MCF-7 RANK OE GFP+Luc+ (RANK OE) cells (n=3-4/group). Mice were treated with human sRANKL 0.5 mg/every 48 h s.c. or vehicle (PBS). Tumors were imaged by bioluminescence every week post tumor inoculation untill the end of the experience (eight weeks). FIG. 10A is a graph of the total flux (p/s) measured at the end of experiment. FIG. 10B is a graph of tumor weight at the time of sacrifice for tumors derived from MCF-7 OE cells taken from mice treated with (+) or without (−) sRANKL. FIG. 10C is a graph of the Ki67-positive stained cells from tumors derived from MCF-7 OE cells taken from mice treated with (+) or without (−) sRANKL. Data is presented as mean±SEM. p-value was calculated using 2-way ANOVA or unpaired t-test, *p<0.05, **p<0.01, ***p<0.001.

FIGS. 11A-11F provide data demonstrating that RANK overexpression increases the level of circulating tumour cells (CTCs). Nod scid gamma (NSG) mice were inoculated in the tail vein with MCF-7GFP+Luc+ (Parental) or MCF-7 RANK OE GFP+Luc+ (RANK OE) cells (n=3/group). Tumours were imaged by bioluminescence 2 h and every week post tumour inoculation untill the end of the experience. FIG. 11A is a series of representative images of bioluminescence of mice inoculated with MCF-7 parental or MCF-7 OE cells at 2 h post-inoculation (p.i.), 2 weeks p.i., 4 weeks p.i., 6 weeks p.i., or 8 weeks p.i. FIG. 11B is a graph of the total flux (p/s) of tumors derived from MCF-7 or MCF-7 OE cells. FIG. 11C is a table of the number of macrometastases assessed by ex vivo bioluminescence and observation at necropsy. FIG. 11D is a graph of the total flux on bone lesion (p/s) and FIG. 11E is a graph of the total flux on lung lesions (p/s). FIG. 11F is a graph of the CTC cells (GFP+ cells) as quantified by flow cytometry. Here, blood was collected by cardiac puncture at sacrifice and CTCs were quantified by flow cytometry. Data analysis was performed using FlowJo V10 software. Data is presented as mean±SEM. p-value was calculated using 2-way ANOVA or unpaired t-test, *p<0.05, **p<0.01, ***p<0.001.

FIGS. 12A-12D provide data demonstrating that Luminal RANK OE tumors are resistant to palbociclib plus fulvestrant in vivo. Nod scid gamma (NSG) mice were inoculated in the 2^{nd} thoracic mammary fat pad with MCF-7GFP+Luc+ (Parental), MCF-7 RANK OE GFP+Luc+(RANK OE) or MCF-7 GFP+Luc+ and MCF-7 RANK OE GFP+Luc+RFP+ cells (1:1) (Mix) (n=3-4/group). Tumors were imaged by bioluminescence every week post tumor inoculation util the end of the experience. Twelve weeks post inoculation mice were randomized into groups based on tumor size and treated with Palbociclib 25 mg/Kg/day p.o. (by oral gavage) plus Fulvestrant 1 mg/day s.c. or vehicle (0,1M Na Lactate plus 95% corn oil, 5% DMSO). FIG. 12A is a graph of the total flux (p/s) for tumors of mice inoculated with Parental, RANK OE, or Mix cells. FIG. 12B is a graph of the tumor weight measured at necropsy. FIG. 12C is a graph of the % Ki67-positive stained cells. FIG. 12D is a graph of relative Cyclin D1 (CCND1) mRNA expression in tumors as determined by RT-qPCR. Ct values were normalized against the 18S gene. Data is presented as mean±SEM. p-value was calculated using 2-way ANOVA or unpaired t-test, *p<0.05, **p<0.01, ***p<0.001.

FIGS. 13A-13D provide data demonstrating that RANK pathway blockade sensitizes TNBC xenografts to palbociclib in vivo. Nod scid gamma (NSG) mice were inoculated bilaterally in the 4^{th} abdominal mammary fat pad with MDA-MB-231 GFP+Luc+ cells (n=2/group). Seven weeks post inoculation mice were randomized based on tumor size and treated with OPG-Fc 10 mg/Kg i.p. twice per week; Palbociclib 25 mg/Kg/day p.o. or the combination. FIG. 13A is a schematic of the experiment design. FIG. 13B is a graph of the tumor weight measured at necropsy. FIG. 13C is an image of cells stained with Ki-67 or phospho-pRb and FIG. 13D is a graph of cells stained positive for Ki67 or phosphor-pRb. Data is presented as mean±SEM. p-value was calculated using ANOVA, *p<0.05, **p<0.01, ***p<0.001.

FIGS. 14A and 14B are schematics of ongoing in vivo models to test triple negative breast cancer (TNBC; FIG. 14A) or estrogen receptor-positive (ER+) breast cancer (FIG. 14B) for sensitivity to CDK4/6 inhibitors (palbociclib) in combination with RANK pathway blockade. Nod scid gamma (NSG) mice were inoculated bilaterally in the 4^{th} abdominal mammary fat pad with MDA-MB-231GFP+Luc+, MCF-7GFP+Luc+ (Parental) or MCF-7 RANK OE GFP+Luc+ (RANK OE) (n=5/group). Tumors are imaged by bioluminescence every week post tumor inoculation till the end of the experience. When tumors reach approximately 100 mm³ mice are randomized based on tumor size and treated according with the experimental protocol.

FIG. 15 is a background illustration depicting the role of RANK in bone remodeling, mammary gland development, mammary carcinogenesis, and adaptive immunity, and how RANK is involved in ER+RANK+ Breast Cancer.

FIGS. 16A-16C depict methodology followed in this study. FIG. 16A is an image describing the makeup of The Cancer Genome Atlas (TCGA) and the numbers of estrogen receptor-positive (ER+) breast cancer (BC) or estrogen receptor-negative (ER−) BC cases within TCGA. Also shown are the numbers of ER+/HER2-negative BC cases of TCGA. FIG. 16B is an illustration of ER+HER2− BC cells contacted with RANK (TNFRSF11A) cDNA packaged into lentiviral vectors to make RANK overexpressing (RANK OE) cells. Measurement of relative RANK mRNA expression is shown in the graph on the right of MCF-7-overexpressing cells (MCF-7^{OE}) T47D overexpressing cells (T470D^{OE}), their parental non-transduced counterparts (MCF-7, T47D), and triple negative breast cancer cells, MDA-MB-231 cells. FIG. 16C is an illustration of the orthotopic mouse model (top) or experimental metastases model (bottom) injected with RANK OE cells or parental cells described in FIG. 16B followed by imaging of mice.

FIGS. 17A-17B provide data that support that in this cohort, high RANK expression associated with decreased 5-year overall survival (OS) and that, although RANK expression was higher in ER-negative breast tumors, there are ˜5% of all ER+ breast cancers with identical RANK expression to the top-25 ER-negative tumors (75% Q). FIG. 17A is a graph of female BC patients from TCGA dichotomized according to RANK expression using the best cut-off (Cu-off Finder software) and survival curves plotted using the Log-rank test. FIG. 17B is a graph of median RANK expression compared between ER-negative and ER-positive BC tumors and within the 75Q of ER-negative tumors. Results are presented as the mean±SEM. P-value was calculated using unapired t-test, *p<0.05, **p<0.01, ***p<0.001.

FIG. 18A are graphs demonstrating the % spheres/seeded cells (left column) and spheroids area (middle column) for MCF-7^{OE}, T47D^{OE} and parental non-transduced counterparts (MCF-7, T47D). In the right column, the graphs depict the area (fold difference from 96 hours vs. 0 hours) upon stimulation with soluble RANKL (sRANKL) for each of the 4 cell types.

FIG. 18B is a graph of expression of the indicated genes associated with chemoresistance, EMT, and stemness for RANK low expressing cells and RANK high expressing cells.

FIG. 19 is a graph depicting the relevance of RANK expression in ER-positive breast cancer and RANK expression in ER-positive tumors may impact on tumor progression.

FIGS. 20A and 20B provide data that support that RANK OE cells are resistant to specific CDK4 inhibitors. Cells were seeded in 96-well plates and exposed to different drugs, 3-ATA (FIG. 20A) or Cdk4 inhibitor Ill (FIG. 20B) for seven days. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001

FIG. 21 provide data that support RANK OE cells are resistant to pan-CDK inhibitor seliciclib and seliciclib does not revert resistance to CDK4/6 inhibitor palbociclib. Cells were seeded in 96-well plates and exposed to different drugs for seven days. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001.

FIGS. 22A and 22B provide data that support RANK expression in cell lines included in this study. (FIG. 22A) RT-qPCR of RANK in parental and RANK OE cell lines (FIG. 22B) Flow cytometry of RANK in parental and RANK OE cell lines

FIG. 23 provide data that support that RANK pathway inhibition with OPG-Fc increases sensitivity of TNBC cell lines to CDK4/6 inhibitors palbociclib, ribociclib and abemaciclib, independently of pRB, PIK3CA, PTEN, BRCA1 and EGFR mutation status. Cells were seeded in 96-well plates and exposed to different drugs for seven days. Medium was replaced every two days, and viability was assessed by Alamar blue assay. Results are the mean of at least three independent assays, with 4 replicates per assay, and presented as the mean±SEM. p-value was calculated using 2-way ANOVA, *p<0.05, **p<0.01, ***p<0.001.

FIGS. 24A and 24B provide data that support that RANK pathway inhibition with OPG-Fc increases sensitivity of TNBC cell lines to CDK4/6 inhibitors palbociclib, ribociclib and abemaciclib, independently of pRB, PIK3CA, PTEN, BRCA1 and EGFR mutation status. Cells were seeded in 6-well plates, exposed to different drugs for six days and allowed to recover for six days in drug-free media. Cells were stained with crystal violet (FIG. 24B), lysed with 1% SDS and media absorbance measured at 570 nM (FIG. 24A). Results are the mean of 3 replicates, and presented as the mean□SEM. p-value was calculated using 2-way ANOVA, *p<0.05, **p<0.01, ***p<0.001

FIG. 25 provide data that support that OPG-Fc has no direct effect on cell proliferation. Cells were seeded in 96-well plates and exposed to OPG-Fc for seven days. Medium was replaced every two days, and viability was assessed by Alamar blue assay. Results are the mean of at least three independent assays, with 4 replicates per assay, and presented as the mean±SEM. p-value was calculated using 1-way ANOVA and considered non-significant if p>0.05

FIGS. 26A and 26B provide data that support that OPG-Fc neutralizes RANKL-induced RANK pathway activation. Protein expression was assessed by western blot. Cells were seeded in 6-well plates, serum starved for 24 h, and exposed to 1 μg/ml RANKL for the indicated time points (FIG. 26A). For RANKL neutralization (FIG. 26B), RANKL was previously incubated for 60 min at 37° C. in serum-depleted medium±100 ng/ml OPG-Fc or 2.5 μg/ml MAB626, and proteins analyzed after 60 min. β-Actin was used as loading control and band intensity was quantified using FiJi

FIG. 27 provides a schematic of an in vivo model to test TNBC sensitivity to CDK4/6 inhibitors (palbociclib) in combination with RANK pathway blockage. Nod scid gama (NSG) mice were inoculated subcutaneously and bilaterally in the flanks (n=5/group). Tumors are measured with caliper. When tumors reach approximately 100 mm³ mice are randomized based on tumor size and treated according with the experimental protocol

FIGS. 28A-28G provide data that support that RANK pathway blockage sensitizes TNBC xenografts to palbociclib in vivo. Nod scid gama (NSG) mice were inoculated subcutaneously and bilaterally in the flanks with MDA-MB-231 cells (n=4-5/group). Six weeks post inoculation mice were randomized based on tumor size and treated with OPG-Fc 10 mg/Kg i.p. 3× per week; Palbociclib 30 mg/Kg/day p.o. or the combination. FIGS. 28A and 28B are graphs of the tumor volume measured with caliper every two days and calculated using the formula Tᵥₒₗo=½(length×width²). FIG. 28C is a graph of the tumor weight at necropsy. FIG. 28D is a graph of the number of mice with metastases after histopathological assessment of organs post necropsy. FIG. 28E is a graph of the osteoclast-specific TRAcP 5b quantified in serum collected at necropsy. FIG. 28F is a pair of graphs showing the Quantification of Ki67 (left) and p-pRb (ImunoRatio) (right). FIG. 28G is a graph of the body weight of mice. Data is presented as mean±SEM. p-value was calculated using ANOVA, *p<0.05, **p<0.01, ***p<0.001.

FIG. 29 provides a schematic of an in vivo model to test luminal RANK OE sensitivity to CDK4/6 inhibitors (palbociclib) plus endocrine therapy (fulvestrant) in combination with RANK pathway blockage. Nod scid gama (NSG) mice were inoculated subcutaneously and bilaterally in the flanks (n=5/group). Tumors are measured with caliper. When tumors reach approximately 100 mm³ mice are randomized based on tumor size and treated according with the experimental protocol

FIG. 30 provides a schematic of an in vivo model to test TNBC sensitivity to CDK4/6 inhibitors (palbociclib) in combination with RANK pathway blockage. Nod scid gama (NSG) mice were inoculated subcutaneously and bilaterally in the flanks (n=5/group). Tumors are measured with caliper. When tumors reach approximately 100 mm³ mice are randomized based on tumor size and treated according with the experimental protocol.

### DETAILED DESCRIPTION

Pharmaceutical Compositions

The present disclosure provides a pharmaceutical composition comprising i) a RANK pathway inhibitor in combination with ii) a CDK inhibitor. In exemplary aspects, the CDK inhibitor is a CDK4/6 inhibitor. Accordingly, the present disclosure provides a pharmaceutical composition comprising i) a RANK pathway inhibitor in combination with ii) a CDK4/6 inhibitor. The present disclosure also provides a pharmaceutical composition comprising i) a RANK pathway inhibitor and ii) a CDK4 inhibitor or a CDK6 inhibitor or both a CDK4 inhibitor and a CDK6 inhibitor.

In exemplary aspects, the CDK inhibitor (e.g., CDK4/6 inhibitor, CDK4 inhibitor, CDK6 inhibitor) is packaged together with the RANK pathway inhibitor. In exemplary aspects, the CDK inhibitor (e.g., CDK4/6 inhibitor, CDK4 inhibitor, CDK6 inhibitor) is formulated together with the RANK pathway inhibitor such that the formulation comprises both of the CDK inhibitor (e.g., CDK4/6 inhibitor, CDK4 inhibitor, CDK6 inhibitor) and the RANK pathway inhibitor and the two ingredients are simultaneously administered upon administration of the formulation. In exemplary aspects, the CDK inhibitor (e.g., CDK4/6 inhibitor, CDK4 inhibitor, CDK6 inhibitor) is packaged separately from the RANK pathway inhibitor. In exemplary aspects, the CDK inhibitor (e.g., CDK4/6 inhibitor, CDK4 inhibitor, CDK6 inhibitor) is formulated separately from the RANK pathway inhibitor such that the CDK inhibitor (e.g., CDK4/6 inhibitor, CDK4 inhibitor, CDK6 inhibitor) may be administered separately from the RANK pathway inhibitor, optionally, the CDK inhibitor (e.g., CDK4/6 inhibitor, CDK4 inhibitor, CDK6 inhibitor) is administered before administration of the RANK pathway inhibitor or the CDK inhibitor (e.g., CDK4/6 inhibitor, CDK4 inhibitor, CDK6 inhibitor) is administered after administration of the RANK pathway inhibitor.

RANK Pathway Inhibitors

As used herein, the term “RANK pathway inhibitor” refers to any compound or molecule that reduces or inhibits the signal transduction that ensues upon the binding of RANKL to RANK. The RANK pathway is reviewed in Boyce and Xing, Current Osteoporosis Reports 5:98-104 (2007) and Darnay et al., TRAFs in RANK Signaling. In: Madame Curie Bioscience Database [Internet]. Austin (Tex.): Landes Bioscience; 2000-2013. Available from: https://www.ncbi.nlm.nih.gov/books/NBK6222/; and Kohli and Kohli, Indian J Endocrinol Metab 15(3): 175-181 (2011), incorporated herein by reference in its entirety. Binding of RANKL to RANK leads to activation of the NFκB, JNK, p38 and/or MAPK signal pathways. RANK, also known as FEO, OFE, ODFR, OSTS, PDB2, TNFRSF11A, CD265, OPTB7, TRANCER and LOH18CR1, is a transmembrane receptor protein expressed on the surface of osteoclasts and precursors thereof. RANK is also expressed by mammary cells and cancer cells (Jones et al., Nature 440: 692-696 (2006); Casimiro et al., PLoS Once 8(5): e63153 (2013), doi: 10.1371/journal.pone.0063153; and Infante et al., J Experimental and Clin Cancer Res 38: 12 (2019). The RANK pathway also mediates migration and invasion of breast and prostate cancer cells (Casimiro et al., 2013, supra). RANK is a member of the tumor necrosis factor (TNF)-receptor superfamily and, like other members of this family, has four extracellular cysteine-rich pseudo-repeat domains (CRDs). The gene encoding RANK is found on human chromosome 18 (arm q21.33) and the amino acid sequence of RANK may be found at the National Center for Biotechnology Information (NCBI) website as Accession No. NP_003830.1 (Isoform 1 precursor), NP_001257878.1 (Isoform 2 precursor), NP_001257879.1 (Isoform 3 precursor), NP_001257880.1 (Isoform 4 precursor), and NP_001265197.1 (Isoform 5 precursor). The amino acid sequence of RANK is set forth herein as SEQ ID NO: 29 and the corresponding mRNA sequence is provided as SEQ ID NO: 30. RANKL, also known as ODF, OPGL, sOdf, CD254, OPTB2, TNFSF11, TNLG6B, TRANCE and hRANKL2, is a member of the TNF cytokine family and is a ligand for osteoprotegerin (OPG) and RANK. RANKL is expressed by stromal cells and osteoblasts, as well as mammary cells. RANKL triggers migration of human epithelial cancer cells and melanoma cells that express RANK (Jones et al., 2006, supra). The gene encoding RANKL is found on human chromosome 13 (arm q14.11) and the amino acid sequence of RANK may be found at the NCBI website as Accession No. NP_003692.1 (Isoform 1) and NP_143026.1 (Isoform 2). The amino acid sequence of RANKL is set forth herein as SEQ ID NO: 31 and the corresponding mRNA sequence is provided as SEQ ID NO: 32. When RANKL binds to RANK, the cytoplasmic domain of RANK interacts with members of the TNF receptor-associated factor (TRAF) family, including TRAF1, TRAF2, TRAF3, TRAF 5, and TRAF6 through one of three cytoplasmic motifs of RANK to activate the NFκB and MAPK signal transduction pathways (Jules et al., JBC 290(39): 23738-23750 (2015)). RANKL binding to RANK leads to polyubiquitination of TRAF6 which recruits TAB2 and in turn leads to activation of TAK1. TAK1 activates downstream kinases leading to activation of NFκB, JNK, and p38 to drive transcription of genes leading to osteoclast differentiation. RANKL binding also leads to activation of the Src kinase pathway through TRAF6 leading to osteoclast activation. TRAF6 also activates signal transduction that leads to NFAT1c translocation into the nucleus where it works with NFκB and AP1. Darnay et al., supra.

As used herein, the terms “inhibit” and “reduce” and words stemming therefrom do not necessarily mean a 100% or complete inhibition or abrogation or reduction. Rather, there are varying degrees of inhibition and/or reduction of which one of ordinary skill in the art recognizes as having a potential benefit or therapeutic effect. In this respect, the RANK pathway inhibitors of the present disclosure may reduce or inhibit the RANK pathway activities to any amount or level. In exemplary embodiments, the reduction or inhibition provided by the RANK pathway inhibitor is at least or about a 10% reduction or inhibition (e.g., at least or about a 20% reduction or inhibition, at least or about a 30% reduction or inhibition, at least or about a 40% reduction or inhibition, at least or about a 50% reduction or inhibition, at least or about a 60% reduction or inhibition, at least or about a 70% reduction or inhibition, at least or about a 80% reduction or inhibition, at least or about a 90% reduction or inhibition, at least or about a 95% reduction or inhibition, at least or about a 98% reduction or inhibition).

In exemplary embodiments, the RANK pathway inhibitor inhibits a binding interaction between RANK and RANK ligand (RANKL). In exemplary aspects, the RANK pathway inhibitor inhibits at least or about 10% of the binding interactions between RANK and RANKL (e.g., at least or about 20% of the binding interactions, at least or about 30% of the binding interactions, at least or about 40% of the binding interactions, at least or about 50% of the binding interactions, at least or about 60% of the binding interactions, at least or about 70% of the binding interactions, at least or about 80% of the binding interactions, at least or about 90% of the binding interactions, at least or about 95% of the binding interactions, at least or about 98% of the binding interactions).

In exemplary aspects, the RANK pathway inhibitor comprises osteoprotegerin (OPG), or a RANKL-binding fragment thereof. OPG, also known as TNFRSF11B, TR1, OCIF, and PDB5, is a protein encoded by a gene located at chromosome 8 (arm q24.12). It is a decoy receptor for RANKL that inhibits osteoclastogenesis. The sequence of OPG is available at the NCBI website as Accession No. NP_002537.3, the mature peptide being amino acids 22-401. In exemplary aspects, the RANK pathway inhibitor comprises the full length OPG molecule (e.g., the mature OPG peptide which is amino acids 22-401 of Accession number NP_002537.3). In various instances, the RANK pathway inhibitor comprises a RANKL-binding fragment and optionally the RANKL-binding fragment comprises only the four cysteine-rich domains of OPG (D1 to D4). In exemplary aspects, the RANK pathway inhibitor is a fusion protein comprising the RANKL-binding fragment (e.g., only the four cysteine-rich domains of OPG (D1 to D4)) fused to an Fc domain of an antibody (optionally, an IgG1 antibody). In exemplary aspects, the RANK pathway inhibitor is similar or identical to the inhibitor described in Body et al., Cancer 97(3 Suppl): 887-892 (2003) and denoted as AMGN-0007. In exemplary aspects, the RANK pathway inhibitor is an OPG-Fc protein, such as the inhibitor available as Catalog Number GQB-21 D1E9 of Genway Biotech Inc. (San Diego, Calif.), Catalog Number P7019F of AB Biosciences (Concord, Mass.), SKU PROT000300-1 of Boster Biological Technology (Pleasanton, Calif.), or Product No. 0 9631 of Sigma (St. Louis, Mo.). In exemplary aspects, the RANK pathway inhibitor is a fusion protein comprising only the four cysteine-rich domains of OPG (D1 to D4)) fused to an Fc domain of an IgG1 antibody.

In exemplary aspects, the RANK pathway inhibitor is an antigen-binding protein that binds to RANK or RANKL. The antigen-binding protein in various aspects is an antibody, an antigen-binding antibody fragment, or an antibody protein product. As used herein, the term “antibody” refers to a protein having a known immunoglobulin format, comprising heavy and light chains, and comprising variable and constant regions. For example, an antibody can be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has variable regions and constant regions. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within the heavy and light chain variable regions where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-delta-, epsilon-, gamma-, or mu-type heavy chain constant regions, e.g., a human alpha-, delta-epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody or a polyclonal antibody. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

An antibody can be cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects of the present disclosure, the antigen-binding protein is an antigen binding fragment of an antibody. As used herein, the term “antigen binding antibody fragment” refers to a portion of an antibody that is capable of binding to the antigen of the antibody and is also known as “antigen-binding fragment” or “antigen-binding portion”. In exemplary instances, the antigen binding antibody fragment is a Fab fragment or a F(ab′)₂ fragment.

In various aspects, the antigen-binding protein is an antibody protein product. As used herein, the term “antibody protein product” refers to any one of several antibody alternatives which in various instances is based on the architecture of an antibody but is not found in nature. In some aspects, the antibody protein product has a molecular-weight within the range of at least about 12-150 kDa. In certain aspects, the antibody protein product has a valency (n) range from monomeric (n=1), to dimeric (n=2), to trimeric (n=3), to tetrameric (n=4), if not higher order valency. Antibody protein products in some aspects are those based on the full antibody structure and/or those that mimic antibody fragments which retain full antigen-binding capacity, e.g., scFvs, Fabs and VHH/VH (discussed below). The smallest antigen binding antibody fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab fragments can be easily produced in host cells, e.g., prokaryotic host cells. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHH/VH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the antigen-binding protein is a bispecific T cell engager (BiTE®) molecule, which is an artificial bispecific monoclonal antibody. BiTE® molecules are fusion proteins comprising two scFvs of different antibodies. One binds to CD3 and the other binds to a target antigen. BiTE® molecules are known in the art. See, e.g., Huehls et al., Immuno Cell Biol 93(3): 290-296 (2015); Rossi et al., MAbs 6(2): 381-91 (2014); Ross et al., PLoS One 12(8): e0183390.

In various aspects, the antigen-binding protein binds to RANKL. The antigen-binding protein in some aspects bind to RANKL in a non-covalent and reversible manner. In exemplary embodiments, the binding strength of the antigen-binding proteins may be described in terms of its affinity, a measure of the strength of interaction between the binding site of the RANK and the RANKL. In exemplary aspects, the antigen-binding proteins have high-affinity for RANKL and thus will bind a greater amount of RANKL in a shorter period of time than low-affinity antigen-binding proteins. In exemplary aspects, the antigen-binding proteins have low-affinity for RANKL and thus will bind a lesser amount of RANKL in a longer period of time than high-affinity antigen-binding proteins. In exemplary aspects, the antigen-binding proteins have an equilibrium association constant, KA, which is at least 10⁵ M⁻¹, at least 10⁶ M⁻¹, at least 10⁷ M⁻¹, at least 10⁸ M⁻¹, at least 10⁹ M⁻¹, or at least 10¹⁰ M⁻¹. As understood by the artisan of ordinary skill, KA can be influenced by factors including pH, temperature and buffer composition.

In exemplary embodiments, the binding strength of the antigen-binding protein to RANKL may be described in terms of its sensitivity. K_{D} is the equilibrium dissociation constant, a ratio of k_{off}/kₒₙ, between the antigen-binding protein and RANKL. K_{D} and KA are inversely related. The K_{D} value relates to the concentration of the antigen-binding protein (the amount of antigen-binding protein needed for a particular experiment) and so the lower the K_{D} value (lower concentration needed) the higher the affinity of the antigen-binding protein. In exemplary aspects, the binding strength of the antigen-binding protein to RANKL may be described in terms of K_{D}. In exemplary aspects, the K_{D} of the antigen-binding proteins is about 10⁻¹ M, about 10⁻² M, about 10⁻³ M, about 10⁻⁴ M, about 10⁻⁵ M, about 10⁻⁶ M, or less. In exemplary aspects, the K_{D} of the antigen-binding protein is micromolar, nanomolar, picomolar or femtomolar. In exemplary aspects, the K_{D} of the antigen-binding proteins is within a range of about 10⁻⁴ to 10⁻⁶ M, or 10⁻⁷ to 10⁻⁹ M, or 10⁻¹⁰ to 10⁻¹² M, or 10⁻¹³ to 10⁻¹⁵ M. In exemplary aspects, the antigen-binding protein binds to the human RANKL with a K_{D} that is greater than or is about 0.04 nM. In exemplary aspects, the antigen-binding protein binds to the human RANKL with a K_{D} of about 0.01 nM to about 20 nM, 0.02 nM to 20 nM, 0.05 nM to 20 nM, 0.05 nM to 15 nM, 0.1 nM to 15 nM, 0.1 nM to 10 nM, 1 nM to 10 nM, or 5 nM to 10 nM.

Optionally, the antigen-binding protein comprises a fully human antibody, a humanized antibody, or a chimeric antibody or a Fab, Fab′, F(ab′)2, or a single chain Fv. In various aspects, the RANK pathway inhibitor comprises one, two, three, four, five or more of the heavy and light chain complementarity determining region (CDR) of an anti-RANK antibody or an anti-RANKL antibody.

In various aspects, the antigen-binding protein binds to RANKL. In some instances, the antigen-binding protein comprises one, two, three, four, five or more of the heavy and light chain complementarity determining region (CDR) of the antibody called denosumab. Denosumab has been described and claimed in International Patent Application No. WO 03/002713 and U.S. Pat. No. 7,364,736, the disclosures of which are hereby incorporated by reference in their entireties. Denosumab comprises a heavy chain CDR1 amino acid sequence of SEQ ID NO: 8, a heavy chain CDR2 amino acid sequence of SEQ ID NO: 9, a heavy chain CDR3 amino acid sequence of SEQ ID NO: 10, a light chain CDR1 amino acid sequence of SEQ ID NO: 5, a light chain CDR2 amino acid sequence of SEQ ID NO: 6, and a light chain CDR3 amino acid sequence of SEQ ID NO: 7. Denosumab comprises a light chain (LC) variable region comprising the amino acid sequence of SEQ ID NO: 1 and a heavy chain (HC) variable region comprising the amino acid sequence of SEQ ID NO: 2. The mature form of the LC is set out as SEQ ID NO: 13, while the mature form of the HC is set out as SEQ ID NO: 14. The full length LC including the signal peptide comprises the amino acid sequence of SEQ ID NO: 3. The full length HC including the signal peptide comprises the amino acid sequence of SEQ ID NO: 4. In various instances, the antigen-binding protein comprises the amino acid sequences of SEQ ID NOs: 5, 6, 7, 8, 9, and 10. In various aspects, the antigen-binding protein comprises an amino acid sequence of SEQ ID NO: 1 and an amino acid sequence of SEQ ID NO: 2. In various aspects, the antigen-binding protein comprises an amino acid sequence of SEQ ID NO: 1 and an amino acid sequence of SEQ ID NO: 2 and further comprises an amino acid sequence of SEQ ID NO: 16 and an amino acid sequence of SEQ ID NO: 28. In various aspects, the antigen-binding protein comprises an amino acid sequence of SEQ ID NO: 13 and an amino acid sequence of SEQ ID NO: 14. In various aspects, the antigen-binding protein comprises an amino acid sequence of SEQ ID NO: 3 and an amino acid sequence of SEQ ID NO: 4.

In various aspects, the antigen-binding protein comprises (a) a heavy chain CDR1 amino acid sequence of SEQ ID NO: 8, or a variant sequence thereof which differs by only one or two amino acids or which has at least or about 70% sequence identity; (b) a heavy chain CDR2 amino acid sequence of SEQ ID NO: 9, or a variant sequence thereof which differs by only one or two amino acids or which has at least or about 70% sequence identity; (c) a heavy chain CDR3 amino acid sequence of SEQ ID NO: 10, or a variant sequence thereof which differs by only one or two amino acids or which has at least or about 70% sequence identity; (d) a light chain CDR1 amino acid sequence of SEQ ID NO: 5, or a variant sequence thereof which differs by only one or two amino acids or which has at least or about 70% sequence identity; (e) a light chain CDR2 amino acid sequence of SEQ ID NO: 6, or a variant sequence thereof which differs by only one or two amino acids or which has at least or about 70% sequence identity; (f) a light chain CDR3 amino acid sequence of SEQ ID NO: 7, or a variant sequence thereof which differs by only one or two amino acids or which has at least or about 70% sequence identity; or (g) a combination of any two or more of (a)-(f). Optionally, the antigen-binding protein comprises (A) a light chain variable domain selected from the group consisting of: (i) a light chain variable domain comprising an amino acid sequence or SEQ ID NO: 1, or a variant sequence which differs by only one or two amino acids or which has at least or about 70% sequence identity to SEQ ID NO: 1; (ii) a light chain variable domain comprising an amino acid sequence encoded by a polynucleotide sequence comprising SEQ ID NO: 19; or (iii) a light chain variable domain comprising an amino acid sequence encoded by a polynucleotide that hybridizes under stringent conditions to the complement of a polynucleotide consisting of SEQ ID NO: 19; or (B) a heavy chain variable domain selected from the group consisting of: (i) a heavy chain variable domain comprising an amino acid of SEQ ID NO: 2, or a variant sequence which differs by only one or two amino acids or which has at least or about 70% sequence identity to SEQ ID NO: 2; (ii) a heavy chain variable domain comprising an amino acid sequence encoded by a polynucleotide sequence comprising SEQ ID NO: 20, or (iii) a heavy chain variable domain comprising an amino acid sequence encoded by a polynucleotide that hybridizes under stringent conditions to the complement of a polynucleotide consisting of SEQ ID NO: 20; or (C) a light chain variable domain of (A) and a heavy chain variable domain of (B). In some aspects, the antigen-binding protein is an IgG1, IgG2, or IgG4 antibody, optionally, comprising a kappa light chain. Optionally, the antigen-binding protein comprises the amino acid sequence of SEQ ID NO: 15 or comprises the amino acid sequence of SEQ ID NO: 16, SEQ ID NO: 17, or SEQ ID NO: 18. In some aspects, the antigen-binding protein comprises: (A) a light chain selected from the group consisting of: (i) a light chain comprising an amino acid sequence of SEQ ID NO: 3 or SEQ ID NO: 13 or a variant sequence which differs by only one or two amino acids or which has at least or about 70% sequence identity to SEQ ID NO: 3 or 13; (ii) a light chain variable domain comprising an amino acid sequence encoded by a polynucleotide sequence comprising SEQ ID NO: 21 or 23; or (iii) a light chain comprising an amino acid sequence encoded by a polynucleotide that hybridizes under stringent conditions to the complement of a polynucleotide consisting of SEQ ID NO: 21 or 23; or (B) a heavy chain selected from the group consisting of: (i) a heavy chain comprising an amino acid sequence of SEQ ID NO: 4 or SEQ ID NO: 14 or a variant sequence which differs by only one or two amino acids or which has at least or about 70% sequence identity to SEQ ID NO: 4 or SEQ ID NO: 14; (ii) a heavy chain variable domain comprising an amino acid sequence encoded by a polynucleotide sequence comprising SEQ ID NO: 22 or 24, or (iii) a heavy chain comprising an amino acid sequence encoded by a polynucleotide that hybridizes under stringent conditions to the complement of a polynucleotide consisting of SEQ ID NO: 22 or 24; or (C) a light chain variable domain of (A) and a heavy chain variable domain of (B). In various instances, the antigen-binding protein comprises the amino acid sequences of SEQ ID NOs: 5, 6, 7, 8, 9, and 10. In various aspects, the antigen-binding protein comprises an amino acid sequence of SEQ ID NO: 1 and an amino acid sequence of SEQ ID NO: 2. In various aspects, the antigen-binding protein comprises an amino acid sequence of SEQ ID NO: 1 and an amino acid sequence of SEQ ID NO: 2 and further comprises an amino acid sequence of SEQ ID NO: 16 and an amino acid sequence of SEQ ID NO: 28. In various aspects, the antigen-binding protein comprises an amino acid sequence of SEQ ID NO: 13 and an amino acid sequence of SEQ ID NO: 14. With regard to the foregoing variant sequences, the variant sequence in some aspects differs by only one or two amino acids or has at least or about 70% sequence identity to the referenced sequence (e.g., SEQ ID NO: 1-10, 13, or 14), at least or about 75% sequence identity to the referenced sequence, at least or about 80% sequence identity to the referenced sequence, at least or about 70% sequence identity to the referenced sequence 85% to the reference SEQ ID NO, at least or about 90% sequence identity to the referenced sequence, at least or about 95% sequence identity to the referenced sequence, or at least or about 98% sequence identity to the referenced sequence.

In exemplary aspects, the antigen-binding protein comprises a fully human antibody, a humanized antibody, or a chimeric antibody or a Fab, Fab′, F(ab′)2, or a single chain Fv, that competes with denosumab for binding to RANKL. In exemplary aspects, the antigen-binding protein binds to an epitope to which denosumab binds. In exemplary aspects, the antigen-binding protein has KD that is similar or the same as denosumab, if not lower. In exemplary instances, the antigen-binding protein that competes with a denosumab for binding to RANKL reduces the amount of denosumab bound to RANKL in an in vitro competitive binding assay. In exemplary aspects, the amount of denosumab bound to RANKL is reduced by at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90% or more. A suitable competitive binding assay that can be used to determine the reduced amount of denosumab bound to RANKL comprises the steps of incubating denosumab with an antigen-binding protein that competes with denosumab for binding to RANKL followed by adding RANKL. The amount of denosumab bound to RANKL is measured with and without the antigen-binding protein that competes for binding to RANKL. Other binding assays, e.g., competitive binding assays or competition assays, which test the ability of an antibody to compete with another antigen-binding protein for binding to an antigen, or to an epitope thereof, are known in the art. See, e.g., U.S. Patent Application Publication No. US20140178905, Chand et al., Biologicals 46: 168-171 (2017); Liu et al., Anal Biochem 525: 89-91 (2017); and Goolia et al., J Vet Diagn Invest 29(2): 250-253 (2017). Also, other methods of comparing two antibodies are known in the art, and include, for example, surface plasmon resonance (SPR). SPR can be used to determine the binding constants of the antibody and second antibody and the two binding constants can be compared.

CDK Inhibitors

In exemplary embodiments, the presently disclosed pharmaceutical composition comprises a RANK pathway inhibitor in combination with a CDK inhibitor. As used herein, the term “CDK inhibitor” means any compound or molecule that targets a cyclin-dependent kinase (CDK). In various instances, the CDK is CDK 1, CDK2, CDK3, CDK4, or CDK6. The CDK in various aspects is CDK4 or CDK6 or a combination thereof. In exemplary instances, the CDK inhibitor is a CDK4/6 inhibitor or a CDK4 inhibitor or a CDK6 inhibitor. The reduction or inhibition provided by the CDK inhibitor may not be a 100% or complete inhibition or abrogation or reduction. Rather, there are varying degrees of reduction or inhibition of which one of ordinary skill in the art recognizes as having a potential benefit or therapeutic effect. In this regard, the CDK inhibitor may inhibit the CDK4 and/or CDK6 protein(s) to any amount or level. In exemplary embodiments, the reduction or inhibition provided by the CDK inhibitor is at least or about 10% reduction or inhibition (e.g., at least or about 20% reduction or inhibition, at least or about 30% reduction or inhibition, at least or about 40% reduction or inhibition, at least or about 50% reduction or inhibition, at least or about 60% reduction or inhibition, at least or about 70% reduction or inhibition, at least or about 80% reduction or inhibition, at least or about 90% reduction or inhibition, at least or about 95% reduction or inhibition, at least or about 98% reduction or inhibition).

Suitable CDK inhibitors are known in the art. See, e.g., Fischer and Gianella-Borradori, Expert Opin. Investig. Drugs (2005) 14(4): 457-477, and International Patent Application Publication Nos. WO2003072062, WO2002096888, WO2003097048, WO2004097048, WO2004069137, and WO2003033499. In exemplary aspects, the CDK inhibitor is alvocidib (also known as flavopiridol; Aventis-NCI), and seliciclib (also known as CYC202, (R)-roscovitine; Cyclacel), UCN-01, Indisulam, BMS-387032, ON01910.Na, AZD-5438, ZK-CDK, JNJ-7706621, GPC-286199.

In exemplary embodiments, the CDK inhibitor is a CDK4/6 inhibitor. As used herein, the term “CDK4/6 inhibitor” refers to any compound or molecule that targets the cyclin-dependent kinases, CDK4 and CDK6, and reduces or inhibits their enzyme activity, e.g., kinase activity. In exemplary aspects, the CDK4/6 inhibitor acts on CDK4 and CDK6 to induce cell-cycle arrest. During cell cycle progression, CDK4 and CDK6 target the growth-suppressive protein, retinoblastoma protein (Rb), for phosphorylation, and the Rb protein is inactivated when phosphorylated. When CDK4 and CDK6 are inhibited by CDK4/6 inhibitors, Rb is not phosphorylated (or is less phosphorylated) such that Rb is free to carry out its growth-suppressive function. In exemplary embodiments, the CDK4/6 inhibitor is a serine/threonine kinase inhibitor, a Cytochrome P450 (CYP450) 3A Inhibitor, or both. In various aspects, the CDK4/6 inhibitor inhibits the phosphorylation of retinoblastoma (Rb) protein. In various aspects, the CDK4/6 inhibitor inhibits the function of CYP4503A.

In exemplary aspects, the CDK4/6 inhibitor comprises a structure:

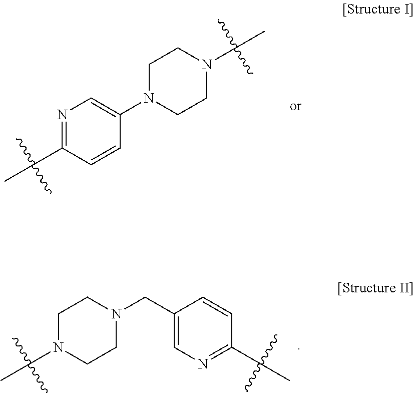 [SMILES: *c1ccc(N2CCN(*)CC2)cn1 ; *c1ccc(CN2CCN(*)CC2)cn1]

In various aspects, the CDK4/6 inhibitor comprises a structure of Structure I or Structure and further comprises a structure of A-B, wherein A comprises a bicyclic structure and B comprises a monocyclic structure. In exemplary aspects, A-B comprises a structure of Structure III or Structure IV or Structure V:

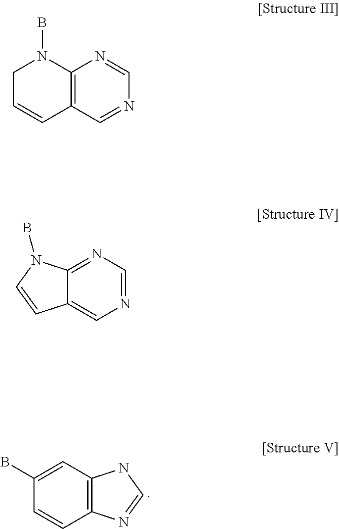

In exemplary aspects, B of Structure III or IV is a cyclopentane. In exemplary aspects, B of Structure V comprises a pyrimidine.

In various aspects, the CDK4/6 inhibitor comprises the structure of

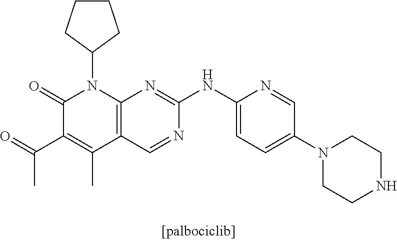 [SMILES: CC(=O)c1c(C)c2cnc(Nc3ccc(N4CCNCC4)cn3)nc2n(C2CCCC2)c1=O]

or a pharmaceutically acceptable salt thereof.

In various aspects, the CDK4/6 inhibitor comprises the structure of

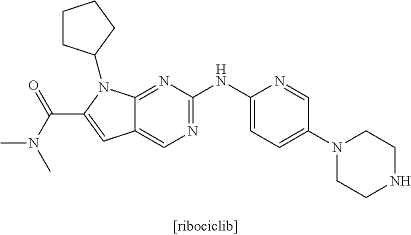 [SMILES: CN(C)C(=O)c1cc2cnc(Nc3ccc(N4CCNCC4)cn3)nc2n1C1CCCC1]

or a pharmaceutically acceptable salt thereof.

In various instances, the CDK4/6 inhibitor comprises the structure of

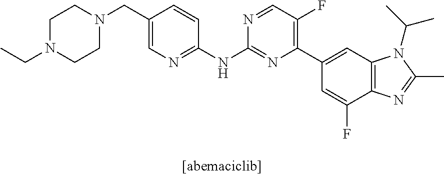 [SMILES: CCN1CCN(Cc2ccc(Nc3ncc(F)c(-c4cc(F)c5nc(C)n(C(C)C)c5c4)n3)nc2)CC1]

or a pharmaceutically acceptable salt thereof.

In exemplary embodiments, the CDK inhibitor of the presently disclosed pharmaceutical composition is a CDK4 inhibitor. Accordingly, in exemplary aspects, the pharmaceutical composition comprises a RANK pathway inhibitor and a CDK4 inhibitor. As used herein, the term “CDK4 inhibitor” refers to any compound or molecule that reduces or inhibits the activity (e.g., kinase activity) of CDK4 but not CDK6. In some aspects, the CDK4 inhibitor is a molecule that targets a nucleic acid encoding CDK4. In exemplary instances, the CDK4 inhibitor is an antisense molecule which mediates RNA interference (RNAi). RNAi is a ubiquitous mechanism of gene regulation in plants and animals in which target mRNAs are degraded in a sequence-specific manner (Sharp, Genes Dev., 15, 485-490 (2001); Hutvagner et al., Curr. Opin. Genet. Dev., 12, 225-232 (2002); Fire et al., Nature, 391, 806-811 (1998); Zamore et al., Cell, 101, 25-33 (2000)). The natural RNA degradation process is initiated by the dsRNA-specific endonuclease Dicer, which promotes cleavage of long dsRNA precursors into double-stranded fragments between 21 and 25 nucleotides long, termed small interfering RNA (siRNA; also known as short interfering RNA) (Zamore, et al., Cell. 101, 25-33 (2000); Elbashir et al., Genes Dev., 15, 188-200 (2001); Hammond et al., Nature, 404, 293-296 (2000); Bernstein et al., Nature, 409, 363-366 (2001)). siRNAs are incorporated into a large protein complex that recognizes and cleaves target mRNAs (Nykanen et al., Cell, 107, 309-321 (2001). The requirement for Dicer in maturation of siRNAs in cells can be bypassed by introducing synthetic 21-nucleotide siRNA duplexes, which inhibit expression of transfected and endogenous genes in a variety of mammalian cells (Elbashir et al., Nature, 411: 494-498 (2001)). In exemplary aspects, the CDK4 inhibitor medicates RNAi and in various instances is a siRNA molecule specific for inhibiting the expression of the nucleic acid (e.g., the mRNA) encoding the CDK4 protein. The term “siRNA” as used herein refers to an RNA (or RNA analog) comprising from about 10 to about 50 nucleotides (or nucleotide analogs) which is capable of directing or mediating RNAi. In exemplary embodiments, a siRNA molecule comprises about 15 to about 30 nucleotides (or nucleotide analogs) or about 20 to about 25 nucleotides (or nucleotide analogs), e.g., 21-23 nucleotides (or nucleotide analogs). The siRNA can be double or single stranded, preferably double-stranded.

In alternative aspects, the CDK4 inhibitor is a short hairpin RNA (shRNA) molecule specific for inhibiting the expression of the nucleic acid (e.g., the mRNA) encoding the CDK4 protein. The term “shRNA” as used herein refers to a molecule of about 20 or more base pairs in which a single-standed RNA partially contains a palindromic base sequence and forms a double-strand structure therein (i.e., a hairpin structure). An shRNA can be a siRNA (or siRNA analog) which is folded into a hairpin structure. shRNAs typically comprise about 45 to about 60 nucleotides, including the approximately 21 nucleotide antisense and sense portions of the hairpin, optional overhangs on the non-loop side of about 2 to about 6 nucleotides long, and the loop portion that can be, e.g., about 3 to 10 nucleotides long. The shRNA can be chemically synthesized. Alternatively, the shRNA can be produced by linking sense and antisense strands of a DNA sequence in reverse directions and synthesizing RNA in vitro with T7 RNA polymerase using the DNA as a template. Though not wishing to be bound by any theory or mechanism, it is believed that after shRNA is introduced into a cell, the shRNA is degraded into a length of about 20 bases or more (e.g., representatively 21, 22, 23 bases), and causes RNAi, leading to an inhibitory effect. Thus, shRNA elicits RNAi and therefore can be used as an effective component of the disclosure. shRNA may preferably have a 3′-protruding end. The length of the double-stranded portion is not particularly limited, but is preferably about 10 or more nucleotides, and more preferably about 20 or more nucleotides. Here, the 3′-protruding end may be preferably DNA, more preferably DNA of at least 2 nucleotides in length, and even more preferably DNA of 2-4 nucleotides in length.

In exemplary aspects, the CDK4 inhibitor is a microRNA (miRNA). As used herein the term “microRNA” refers to a small (e.g., 15-22 nucleotides), non-coding RNA molecule which base pairs with mRNA molecules to silence gene expression via translational repression or target degradation. microRNA and the therapeutic potential thereof are described in the art. See, e.g., Mulligan, MicroRNA: Expression, Detection, and Therapeutic Strategies, Nova Science Publishers, Inc., Hauppauge, N.Y., 2011; Bader and Lammers, “The Therapeutic Potential of microRNAs” Innovations in Pharmaceutical Technology, pages 52-55 (March 2011).

In exemplary embodiments, the CDK inhibitor of the presently disclosed pharmaceutical composition is a CDK6 inhibitor. Accordingly, in exemplary aspects, the pharmaceutical composition comprises a RANK pathway inhibitor and a CDK6 inhibitor. As used herein, the term “CDK6 inhibitor” refers to any compound or molecule that reduces or inhibits the activity (e.g., kinase activity) of CDK6 but not CDK4. In some aspects, the CDK6 inhibitor is a molecule that targets a nucleic acid encoding CDK6. In exemplary instances, the CDK6 inhibitor is an antisense molecule which mediates RNA interference (RNAi). In exemplary aspects, the CDK6 inhibitor mediates RNAi and in various instances is a siRNA molecule specific for inhibiting the expression of the nucleic acid (e.g., the mRNA) encoding the CDK6 protein. In alternative aspects, the CDK6 inhibitor is alternatively a short hairpin RNA (shRNA) molecule specific for inhibiting the expression of the nucleic acid (e.g., the mRNA) encoding the CDK6 protein. In exemplary aspects, the CDK6 inhibitor is a microRNA (miRNA).

Additional Active Ingredients

In various instances, the pharmaceutical composition comprises more than one type of RANK pathway inhibitor and/or more than one type of CDK inhibitor (e.g., CDK4/6 inhibitor, CDK4 inhibitor, CDK6 inhibitor). For instance, the pharmaceutical composition comprises at least two of OPG, OPG-Fc, and denosumab, and at least two of palbociclib, ribociclib and abemaciclib. In various instances, the pharmaceutical composition comprises additional active ingredients other than a RANK pathway inhibitor and other than a CDK inhibitor (e.g., CDK4/6 inhibitor, CDK4 inhibitor, CDK6 inhibitor). In exemplary instances, the pharmaceutical composition comprises a RANK pathway inhibitor in combination with a CDK inhibitor (e.g., CDK4/6 inhibitor) and a chemotherapeutic agent.

Chemotherapeutic agents suitable for inclusion in the presently disclosed pharmaceutical compositions are known in the art, and include, but not limited to, platinum coordination compounds, topoisomerase inhibitors, antibiotics, antimitotic alkaloids and difluoronucleosides, as described in U.S. Pat. No. 6,630,124.

In some embodiments, the chemotherapeutic agent is a platinum coordination compound. The term “platinum coordination compound” refers to any tumor cell growth inhibiting compound that provides a platinum in the form of an ion. In some embodiments, the platinum coordination compound is cis-diamminediaquoplatinum (II)-ion; chloro(diethylenetriamine)-platinum(II)chloride; dichloro(ethylenediamine)-platinum(II), diammine(1,1-cyclobutanedicarboxylato) platinum(II) (carboplatin); spiroplatin; iproplatin; diammine(2-ethylmalonato)-platinum(II); ethylenediaminemalonatoplatinum(II); aqua(1,2-diaminodyclohexane)-sulfatoplatinum(II); (1,2-diaminocyclohexane)malonatoplatinum(II); (4-caroxyphthalato)(1,2-diaminocyclohexane)platinum(II); (1,2-diaminocyclohexane)-(isocitrato)platinum(II); (1,2-diaminocyclohexane)cis(pyruvato)platinum(II); (1,2-diaminocyclohexane)oxalatoplatinum(II); ormaplatin; and tetraplatin.

In some embodiments, cisplatin is the platinum coordination compound employed in the compositions and methods of the present invention. Cisplatin is commercially available under the name PLATINOL™ from Bristol Myers-Squibb Corporation and is available as a powder for constitution with water, sterile saline or other suitable vehicle. Other platinum coordination compounds suitable for use in the present invention are known and are available commercially and/or can be prepared by known techniques. Cisplatin, or cis-dichlorodiammineplatinum II, has been used successfully for many years as a chemotherapeutic agent in the treatment of various human solid malignant tumors. More recently, other diamino-platinum complexes have also shown efficacy as chemotherapeutic agents in the treatment of various human solid malignant tumors. Such diamino-platinum complexes include, but are not limited to, spiroplatinum and carboplatinum. Although cisplatin and other diamino-platinum complexes have been widely used as chemotherapeutic agents in humans, they have had to be delivered at high dosage levels that can lead to toxicity problems such as kidney damage.

In some embodiments, the chemotherapeutic agent is a topoisomerase inhibitor. Topoisomerases are enzymes that are capable of altering DNA topology in eukaryotic cells. Topoisomerases are critical for cellular functions and cell proliferation. Generally, there are two classes of topoisomerases in eukaryotic cells, type I and type II. Topoisomerase I is a monomeric enzyme of approximately 100,000 molecular weight. The enzyme binds to DNA and introduces a transient single-strand break, unwinds the double helix (or allows it to unwind), and subsequently reseals the break before dissociating from the DNA strand. Various topoisomerase inhibitors have been shown clinical efficacy in the treatment of humans afflicted with ovarian cancer, breast cancer, esophageal cancer or non-small cell lung carcinoma.

In some aspects, the topoisomerase inhibitor is camptothecin or a camptothecin analog. Camptothecin is a water-insoluble, cytotoxic alkaloid produced by Camptotheca accuminata trees indigenous to China and Nothapodytes foetida trees indigenous to India. Camptothecin inhibits growth of a number of tumor cells. Compounds of the camptothecin analog class are typically specific inhibitors of DNA topoisomerase I. Compounds of the camptothecin analog class include, but are not limited to; topotecan, irinotecan and 9-amino-camptothecin.

In additional embodiments, the chemotherapeutic agent is any tumor cell growth inhibiting camptothecin analog claimed or described in: U.S. Pat. No. 5,004,758, issued on Apr. 2, 1991 and European Patent Application Number 88311366.4, published on Jun. 21, 1989 as 20′ Publication Number EP 0 321 122; U.S. Pat. No. 4,604,463, issued on Aug. 5, 1986 and European Patent Application Publication Number EP 0 137 145, published on Apr. 17, 1985; U.S. Pat. No. 4,473,692, issued on Sep. 25, 1984 and European Patent Application Publication Number EP 0 074 256, published on Mar. 16, 1983; U.S. Pat. No. 4,545,880, issued on Oct. 8, 1985 and European Patent Application Publication Number EP 0 074 256, published on Mar. 16, 1983; European Patent Application Publication Number EP 0 088 642, published on Sep. 14, 1983; Wani et al., J. Med. Chem., 29, 2358-2363 (1986); Nitta et al., Proc. 14th International Congr. Chemotherapy, Kyoto, 1985, Tokyo Press, Anticancer Section 1, p. 28-30, especially a compound called CPT-11. CPT-11 is a camptothecin analog with a 4-(piperidino)-piperidine side chain joined through a carbamate linkage at C-10 of 10-hydroxy-7-ethyl camptothecin. CPT-11 is currently undergoing human clinical trials and is also referred to as irinotecan; Wani et al, J. Med. Chem., 23, 554 (1980); Wani et. al., J. Med. Chem., 30, 1774 (1987); U.S. Pat. No. 4,342,776, issued on Aug. 3, 1982; U.S. patent application Ser. No. 581,916, filed on Sep. 13, 1990 and European Patent Application Publication Number EP 418 099, published on Mar. 20, 1991; U.S. Pat. No. 4,513,138, issued on Apr. 23, 1985 and European Patent Application Publication Number EP 0 074 770, published on Mar. 23, 1983; U.S. Pat. No. 4,399,276, issued on Aug. 16, 1983 and European Patent Application Publication Number 0 056 692, published on Jul. 28, 1982; the entire disclosure of each of which is hereby incorporated by reference. All of the above-listed compounds of the camptothecin analog class are available commercially and/or can be prepared by known techniques including those described in the above-listed references. The topoisomerase inhibitor may be selected from the group consisting of topotecan, irinotecan and 9-aminocamptothecin.

The preparation of numerous compounds of the camptothecin analog class (including pharmaceutically acceptable salts, hydrates and solvates thereof) as well as the preparation of oral and parenteral pharmaceutical compositions comprising such a compounds of the camptothecin analog class and an inert, pharmaceutically acceptable carrier or diluent, is extensively described in U.S. Pat. No. 5,004,758, issued on Apr. 2, 1991 and European Patent Application Number 88311366.4, published on Jun. 21, 1989 as Publication Number EP 0 321 122, the teachings of which are incorporated herein by reference.

In still yet other embodiments, the chemotherapeutic agent is an antibiotic compound. Suitable antibiotic include, but are not limited to, doxorubicin, mitomycin, bleomycin, daunorubicin and streptozocin.

In some embodiments, the chemotherapeutic agent is an antimitotic alkaloid. In general, antimitotic alkaloids can be extracted from Cantharanthus roseus, and have been shown to be efficacious as anticancer chemotherapy agents. A great number of semi-synthetic derivatives have been studied both chemically and pharmacologically (see, O. Van Tellingen et al, Anticancer Research, 12, 1699-1716 (1992)). The antimitotic alkaloids of the present invention include, but are not limited to, vinblastine, vincristine, vindesine, paclitaxel (PTX; Taxol®) and vinorelbine. The latter two antimitotic alkaloids are commercially available from Eli Lilly and Company, and Pierre Fabre Laboratories, respectively (see, U.S. Pat. No. 5,620,985). In an exemplary aspect of the present invention, the antimitotic alkaloid is vinorelbine.

In other embodiments of the invention, the chemotherapeutic agent is a difluoronucleoside. 2′-deoxy-2′,2′-difluoronucleosides are known in the art as having antiviral activity. Such compounds are disclosed and taught in U.S. Pat. Nos. 4,526,988 and 4,808,614. European Patent Application Publication 184,365 discloses that these same difluoronucleosides have oncolytic activity. In certain specific aspects, the 2′-deoxy-2′,2′-difluoronucleoside used in the compositions and methods of the present invention is 2′-deoxy-2′,2′-difluorocytidine hydrochloride, also known as gemcitabine hydrochloride. Gemcitabine is commercially available or can be synthesized in a multi-step process as disclosed and taught in U.S. Pat. Nos. 4,526,988, 4,808,614 and 5,223,608, the teachings of which are incorporated herein by reference.

In exemplary aspects, the chemotherapeutic agent is a hormone therapy agent. In exemplary aspects, the pharmaceutical composition comprises a RANK pathway inhibitor in combination with a CDK inhibitor and further comprises a hormone therapy agent. In exemplary instances, the hormone therapy agent is, for instance, letrozole, tamoxifen, bazedoxifene, exemestane, leuprolide, goserelin, fulvestrant, anastrozole, or toremifene. In exemplary aspects, the hormone therapy agent is a luteinizing hormone (LH) blocker, e.g., gosarelin, or an LH releasing hormone (RH) agonist. In exemplary aspects, the hormone therapy agent is an ER-targeted agent (e.g., fulvestrant or tamoxifen), rapamycin, a rapamycin analog (e.g., everolimus, temsirolimus, ridaforolimus, zotarolimus, and 32-deoxo-rapamycin), an anti-HER2 drug (e.g., trastuzumab, pertuzumab, lapatinib, T-DM1, or neratinib) or a PI3K inhibitor (e.g., taselisib, alpelisib or buparlisib).

In exemplary aspects, the pharmaceutical composition comprises a RANK pathway inhibitor in combination with a CDK4/6 inhibitor and further comprises any additional active ingredient combined with a CDK4/6 inhibitor, as described in Knudsen and Witkiewicz, Trends Cancer 3(1): 39-55 (2017). Table 3 of this reference describes many drug combinations in clinical trials wherein at least one active ingredient is a CDK4/6 inhibitor. In exemplary aspects, the pharmaceutical composition comprises a RANK pathway inhibitor in combination with a CDK4/6 inhibitor and further comprises any one or more of: AZD-2014, BYL719, BKM120, everolimus, PI3K inhibitor, T-DM1, paclitaxel, TACE, radiation therapy, Docetaxel, Nab, Carboplatin, Cisplatin, 5FU, Oxaliplatin, Cetuximab, trastuzumab, MEK162, androgen deprivation, Enzalutamide, ibrutinib, ceritinib, MEK inhibitor, trametinib, HDM201, and/or an MTOR inhibitor.

In exemplary aspects, the pharmaceutical composition comprises a RANK pathway inhibitor in combination with a CDK4/6 inhibitor and further comprises an active ingredient listed on the package insert of a CDK4/6 inhibitor approved by the U.S. Food and Drug Administration (FDA) or other drug regulatory agency, e.g., European Medicines Agency (EMEA), Health Canada, Therapeutic Goods Administration (TGA), State Food and Drug Administration of China, Ministry of Health, Labour and Welfare of (MHLW) Japan. Accordingly, the pharmaceutical composition in some aspects, comprises a RANK pathway inhibitor in combination with a CDK4/6 inhibitor (e.g., palbociclib, ribociclib, abemaciclib) and further comprises fulvestrant or an aromatase inhibitor (e.g., letrozole, anastrozole, or exemestante). In various instances, the pharmaceutical composition comprises abemaciclib in combination with fulvestrant or palbociblib and fulvestrant or ribociclib and fulvestrant or palbociblib and an aromatase inhibitor or ribociclib and an aromatase inhibitor.

Pharmaceutically Acceptable Salts

In exemplary aspects, the presently disclosed pharmaceutical composition comprises a RANK pathway inhibitor and a CDK4/6 inhibitor as active ingredients, or a pharmaceutically acceptable salt thereof. Such salts can be prepared in situ during the final isolation and purification of the active ingredient or separately prepared by reacting a free base function with a suitable acid. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate.

Basic addition salts also can be prepared in situ during the final isolation and purification of the active agent, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like.

Further, basic nitrogen-containing groups can be quaternized with such active agents as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

Pharmaceutically Acceptable Carriers

In exemplary embodiments, the RANK pathway inhibitor is formulated with a pharmaceutically acceptable carrier, diluent, or excipient. In exemplary embodiments, the CDK4/6 inhibitor is formulated with a pharmaceutically acceptable carrier, diluent, or excipient. Depending on the route of administration, the particular active agent intended for use (RANK pathway inhibitor or CDK4/6 inhibitor), as well as other factors, the RANK pathway inhibitor or CDK4/6 inhibitor may be admixed with one or more additional pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents.

In exemplary aspects, the RANK pathway inhibitor is formulated for subcutaneous injection. In exemplary aspects, the RANK pathway inhibitor is formulated with sorbitol, acetate, polysorbate 20, water for injection and sodium hydroxide to a pH of 5.2. Optionally, the RANK pathway inhibitor is formulated with 4.7% sorbitol, 17 mM acetate, 0.01% polysorbate 20, water for injection and sodium hydroxide to a pH of 5.2. In exemplary aspects, the RANK pathway inhibitor is denosumab and the pharmaceutical composition optionally comprises 60 mg denosumab formulated with 4.7% sorbitol, 17 mM acetate, 0.01% polysorbate 20, water for injection and sodium hydroxide to a pH of 5.2 In exemplary aspects, the RANK pathway inhibitor is denosumab and the pharmaceutical composition optionally comprises 120 mg denosumab formulated with 4.6% sorbitol, 18 mM acetate, 0.01% polysorbate 20, water for injection and sodium hydroxide to a pH of 5.2. In exemplary aspects, the pharmaceutical composition comprises Prolia® or Xgeva®, or a biosimilar thereof.

In exemplary aspects, the CDK4/6 inhibitor is formulated for oral administration. In various instances, the CDK4/6 inhibitor is formulated into a capsule. In exemplary aspects, the CDK4/6 inhibitor is formulated with microcrystalline cellulose, lactose monohydrate, sodium starch glycolate, colloidal silicon dioxide, magnesium stearate, and hard gelatin capsule shells. The light orange, light orange/caramel, and caramel opaque capsule shells contain gelatin, red iron oxide, yellow iron oxide, and titanium dioxide; the printing ink contains shellac, titanium dioxide, ammonium hydroxide, propylene glycol, and simethicone. In exemplary aspects, the CDK4/6 inhibitor is palbociclib and optionally 125 mg, 100 mg, or 75 mg palbociblib is formulated with microcrystalline cellulose, lactose monohydrate, sodium starch glycolate, colloidal silicon dioxide, magnesium stearate, and hard gelatin capsule shells. The light orange, light orange/caramel, and caramel opaque capsule shells contain gelatin, red iron oxide, yellow iron oxide, and titanium dioxide; the printing ink contains shellac, titanium dioxide, ammonium hydroxide, propylene glycol, and simethicone. In exemplary aspects, the CDK4/6 inhibitor is Ibrance®, or a generic version thereof.

In exemplary aspects, the CDK4/6 inhibitor is formulated for oral administration. In various instances, the CDK4/6 inhibitor is formulated into a tablet. In exemplary aspects, the CDK4/6 inhibitor is formulated with colloidal silicon dioxide, crospovidone, hydroxypropylcellulose, magnesium stearate and microcrystalline cellulose. In exemplary aspects, the CDK4/6 inhibitor is ribociclib and optionally 200 mg ribociblib is formulated with colloidal silicon dioxide, crospovidone, hydroxypropylcellulose, magnesium stearate and microcrystalline cellulose. Optionally, the tablet comprises a film coating and the film coating optionally comprises iron oxide black, iron oxide red, lecithin (soya), polyvinyl alcohol (partially hydrolysed), talc, titanium dioxide, and xanthan gum as inactive ingredients. In exemplary aspects, the CDK4/6 inhibitor is Kisqali®, or a generic version thereof.

In exemplary aspects, the CDK4/6 inhibitor is formulated for oral administration. In various instances, the CDK4/6 inhibitor is formulated into a tablet. In exemplary aspects, the CDK4/6 inhibitor is formulated with microcrystalline cellulose 102, microcrystalline cellulose 101, lactose monohydrate, croscarmellose sodium, sodium stearyl fumarate, silicon dioxide. In exemplary aspects, the tablets comprise the color mixture ingredients, polyvinyl alcohol, titanium dioxide, polyethylene glycol, talc, iron oxide yellow, and iron oxide red. In exemplary aspects, the CDK4/6 inhibitor is Verzenio®, or a generic version thereof.

In exemplary aspects, the pharmaceutical composition comprises an additional active ingredient and that active ingredient is fulvestrant. In exemplary aspects, fulvestrant is formulated for injection, optionally, intramuscular injection. In various instances, fulvestrant is formulated with 10% w/v Alcohol, USP, 10% w/v Benzyl Alcohol, NF, and 15% w/v Benzyl Benzoate, USP, as co-solvents, and made up to 100% w/v with Castor Oil, USP as a co-solvent and release rate modifier. In exemplary aspects, 500 mg fulvestrant is formulated with 10% w/v Alcohol, USP, 10% w/v Benzyl Alcohol, NF, and 15% w/v Benzyl Benzoate, USP, as co-solvents, and made up to 100% w/v with Castor Oil, USP as a co-solvent and release rate modifier. In exemplary aspects, the pharmaceutical composition comprises Faslodex®, or a generic version thereof.

In exemplary aspects, the pharmaceutical composition comprises an additional active ingredient and that active ingredient is everolimus. In exemplary aspects, everolimus is formulated for oral administration, optionally, as a tablet. In various instances, everolimus, optionally, 2.5 mg, 5 mg, or 10 mg of everolimus, is formulated with butylated hydroxytoluene, magnesium stearate, lactose monohydrate, hypromellose, crospovidone, and lactose anhydrous as inactive ingredients. In exemplary aspects, the pharmaceutical composition comprises Afinitor®, or a generic version thereof.

Kits

The present disclosure additionally provides kits comprising any one of the presently disclosed pharmaceutical compositions. The kit in exemplary embodiments comprises a RANK pathway inhibitor and a CDK inhibitor. The kit in exemplary embodiments comprises a RANK pathway inhibitor and a CDK4/6 inhibitor, CDK4 inhibitor, or CDK6 inhibitor. The RANK pathway inhibitor may be any of those described herein, e.g., OPG, OPG-Fc, denosumab. The CDK4/6 inhibitor may be any of those described herein, e.g., abemaciclib, palbociclib, ribociclib. In exemplary aspects, the RANK pathway inhibitor is formulated for subcutaneous injection. In exemplary aspects, the RANK pathway inhibitor is formulated with sorbitol, acetate, polysorbate 20, water for injection and sodium hydroxide to a pH of 5.2 Optionally, the RANK pathway inhibitor is formulated with 4.7% sorbitol, 17 mM acetate, 0.01% polysorbate 20, water for injection and sodium hydroxide to a pH of 5.2. Alternatively, the RANK pathway inhibitor is formulated with 4.6% sorbitol, 18 mM acetate, 0.01% polysorbate 20, water for injection and sodium hydroxide to a pH of 5.2. In exemplary aspects, the RANK pathway inhibitor is provided as a single-use vial or as a single-use prefilled syringe. In exemplary aspects, the CDK4/6 inhibitor is formulated for oral administration, optionally, a tablet or a capsule. In exemplary instances, the CDK4/6 inhibitor is formulated with formulated with microcrystalline cellulose, lactose monohydrate, sodium starch glycolate, colloidal silicon dioxide, magnesium stearate, and hard gelatin capsule shells. The light orange, light orange/caramel, and caramel opaque capsule shells contain gelatin, red iron oxide, yellow iron oxide, and titanium dioxide; the printing ink contains shellac, titanium dioxide, ammonium hydroxide, propylene glycol, and simethicone. In exemplary aspects, the CDK4/6 inhibitor is Ibrance®, or a generic version thereof. In exemplary instances, the CDK4/6 inhibitor is formulated with colloidal silicon dioxide, crospovidone, hydroxypropylcellulose, magnesium stearate and microcrystalline cellulose. In exemplary aspects, the CDK4/6 inhibitor is Kisqali®, or a generic version thereof. In exemplary aspects, the CDK4/6 inhibitor is formulated with microcrystalline cellulose 102, microcrystalline cellulose 101, lactose monohydrate, croscarmellose sodium, sodium stearyl fumarate, silicon dioxide. In exemplary aspects, the CDK4/6 inhibitor is Verzenio®, or a generic version thereof.

Responsiveness, Sensitivity and Resistance

Without being bound to any particular theory, the presently disclosed pharmaceutical compositions are useful for increasing responsiveness of a cancer or a tumor (also referred to herein as a lesion) to a CDK inhibitor, e.g., CDK4/6 inhibitor. Accordingly, the present disclosure provides methods of increasing responsiveness of a cancer cell or tumor cell to treatment with a CDK inhibitor, e.g., CDK4/6 inhibitor. In exemplary embodiments, the method comprises administering to the subject a RANK pathway inhibitor in combination with a CDK inhibitor, e.g., CDK4/6 inhibitor. In exemplary embodiments, the subject is or has been treated with a CDK inhibitor, e.g., CDK4/6 inhibitor, and the method comprises administering a RANK pathway inhibitor to the subject.

The term “responsiveness” as used herein refers to the extent of a therapeutic response or responsiveness of a cancer cell or tumor to a drug/compound (e.g., a CDK4/6 inhibitor) or other treatment (e.g., radiation therapy) as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), in order to provide a baseline for comparison after treatment with a drug (e.g., CDK4/6 inhibitor). The response assessment and evaluation criteria for tumors are published in Eisenhauer et al., Eur J Cancer 45:228-247 (2009) and Litière et al., Journal of Clinical Oncology 37(13): 1102-1110 (2019) DOI: 10.1200/JCO.18.01100. Briefly, Section 4.3 of Eisenhauer et al., 2009, supra, teaches response criteria to be used to determine objective tumor response for target lesions, as follows:

| Response Type | Signifies that: |
|---|---|
| Complete | Disappearance of all target lesions. Any pathological |
| Response (CR) | lymph nodes (whether target or non-target) must |
| | have reduction in short axis to < 10 mm. |
| Partial | At least a 30% decrease in the sum of diameters of |
| Response (PR) | target lesions, taking as reference the baseline sum |
| | diameters. |
| Stable | Neither sufficient shrinkage to qualify for PR nor |
| Disease (SD) | sufficient increase to qualify for PD, taking as |
| | reference the smallest sum diameters while on study |
| Progressive | At least a 20% increase in the sum of diameters of |
| Disease (PD) | target lesions, taking as reference the smallest sum |
| | on study (this includes the baseline sum if that is the |
| | smallest on study). In addition to the relative increase |
| | of 20%, the sum must also demonstrate an absolute |
| | increase of at least 5 mm. (Note: the appearance of |
| | one or more new lesions is also considered progression.) |

In ideal cases, a drug or other treatment results in CR or PR. Responses of SD or PD in some aspects are used to show that a drug is not an effective treatment for cancer or that a tumor has stopped responding to treatment.

The increase in responsiveness of a tumor or cancer cell to a CDK inhibitor, e.g., CDK4/6 inhibitor, provided by the methods of the present disclosure may be at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to a control. The increase in responsiveness of a tumor cell or cancer cell to a CDK inhibitor, e.g., CDK4/6 inhibitor, provided by the methods of the present disclosure may be at least or about a 10% to greater than about a 95% increase (e.g., at least or about a 10% increase, at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase, at least or about a 100% increase) relative to a control. In exemplary aspects, the control is cancer or tumor or a subject or a population of subjects that was not treated with the presently disclosed pharmaceutical composition or wherein the subject was treated with a placebo.

In exemplary aspects, the increase in responsiveness to a CDK inhibitor, e.g., CDK4/6 inhibitor, is achieved for a tumor or cancer cell that was previously treated with and responsive to the CDK inhibitor (e.g., CDK4/6 inhibitor) but, over time, lost responsiveness to the CDK inhibitor (e.g., CDK4/6 inhibitor). In exemplary aspects, the tumor or cancer cell was treated with a CDK inhibitor (e.g., CDK4/6 inhibitor) for a first time period and, during the first time period, the tumor or cancer cell was responsive to treatment with the CDK inhibitor (e.g., CDK4/6 inhibitor), but during a second time period occurring after the first time period, the tumor or cancer cell became less responsive to treatment with the CDK inhibitor (e.g., CDK4/6 inhibitor). As recognized by one of ordinary skill in the art, such a tumor or cancer cell is understood as one that has lost sensitivity to treatment and/or one that has become resistant to treatment. In exemplary embodiments, the presently disclosed methods increase the responsiveness of the tumor or cancer cell to the CDK inhibitor (e.g., CDK4/6 inhibitor) to the level of responsiveness as observed during the first time period. In such cases, it is understood that the methods restore the responsiveness of the tumor (or lesion) or cancer cell to the CDK inhibitor (e.g., CDK4/6 inhibitor). The term “restore” means reinstate or return to a previous state (e.g., a state of responsiveness to treatment). Accordingly, provided herein are methods of restoring responsiveness of a tumor or cancer cell to treatment with a CDK inhibitor (e.g., CDK4/6 inhibitor).

The present disclosure provides methods of increasing or restoring sensitivity of a cancer cell or tumor to treatment with a CDK inhibitor, e.g., CDK4/6 inhibitor. In exemplary embodiments, the method comprises administering to the subject a RANK pathway inhibitor in combination with a CDK inhibitor, e.g., CDK4/6 inhibitor. In exemplary embodiments, the subject is or has been treated with a CDK inhibitor, e.g., CDK4/6 inhibitor, and the method comprises administering a RANK pathway inhibitor to the subject. As used herein “sensitivity” refers to the way a tumor reacts to a drug/compound, e.g., a CDK inhibitor (e.g., CDK4/6 inhibitor). In exemplary aspects, “sensitivity” means “responsive to treatment” and the concepts of “sensitivity” and “responsiveness” are positively associated in that a tumor or cancer cell that is responsive to a drug/compound treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistant” and the concept of “resistance” is negatively associated with “sensitivity”. For example, a tumor that is resistant to a drug treatment is neither sensitive nor responsive to that drug, and that drug is not an effective treatment for that tumor or cancer cell. The increase in sensitivity provided by the methods of the present disclosure may be at least or about a 1% to about a 10% increase (e.g., at least or about a 1% increase, at least or about a 2% increase, at least or about a 3% increase, at least or about a 4% increase, at least or about a 5% increase, at least or about a 6% increase, at least or about a 7% increase, at least or about a 8% increase, at least or about a 9% increase, at least or about a 9.5% increase, at least or about a 9.8% increase, at least or about a 10% increase) relative to a control. The increase in sensitivity provided by the methods of the present disclosure may be at least or about a 10% to greater than about a 95% increase (e.g., at least or about a 10% increase, at least or about a 20% increase, at least or about a 30% increase, at least or about a 40% increase, at least or about a 50% increase, at least or about a 60% increase, at least or about a 70% increase, at least or about a 80% increase, at least or about a 90% increase, at least or about a 95% increase, at least or about a 98% increase, at least or about a 100% increase) relative to a control. In exemplary aspects, the control is cancer or tumor or a subject or a population of subjects that was not treated with the presently disclosed pharmaceutical composition or wherein the subject or population of subjects was treated with a placebo.

Also provided herein are methods of decreasing resistance of a cancer cell or tumor cell to treatment with a CDK inhibitor, e.g., CDK4/6 inhibitor. In exemplary embodiments, the method comprises administering to the subject a RANK pathway inhibitor in combination with a CDK inhibitor, e.g., CDK4/6 inhibitor. In exemplary embodiments, the subject is or has been treated with a CDK inhibitor, e.g., CDK4/6 inhibitor, and the method comprises administering a RANK pathway inhibitor to the subject. The decrease in resistance of a tumor cell or cancer cell to a CDK inhibitor, e.g., CDK4/6 inhibitor, provided by the methods of the present disclosure may be at least or about a 1% to about a 10% decrease (e.g., at least or about a 1% decrease, at least or about a 2% decrease, at least or about a 3% decrease, at least or about a 4% decrease, at least or about a 5% decrease, at least or about a 6% decrease, at least or about a 7% decrease, at least or about a 8% decrease, at least or about a 9% decrease, at least or about a 9.5% decrease, at least or about a 9.8% decrease, at least or about a 10% decrease) relative to a control. The decrease in resistance of a tumor cell or cancer cell to a CDK inhibitor, e.g., CDK4/6 inhibitor, provided by the methods of the present disclosure may be at least or about a 10% to greater than a 95% decrease (e.g., at least or about a 10% decrease, at least or about a 20% decrease, at least or about a 30% decrease, at least or about a 40% decrease, at least or about a 50% decrease, at least or about a 60% decrease, at least or about a 70% decrease, at least or about a 80% decrease, at least or about a 90% decrease, at least or about a 95% decrease, at least or about a 98% decrease, at least or about a 100% decrease) relative to a control. In exemplary aspects, the control is cancer or tumor or a subject or a population of subjects that was not treated with the presently disclosed pharmaceutical composition or wherein the subject or population of subjects was treated with a placebo.

Treatment

Additionally provided herein are methods of treating cancer in a subject. In exemplary embodiments, the method comprises administering to the subject a RANK pathway inhibitor optionally in combination with a CDK inhibitor, e.g., CDK4/6 inhibitor. In exemplary embodiments, the subject is or has been treated with a CDK inhibitor, e.g., CDK4/6 inhibitor and the method comprises administering to the subject a RANK pathway inhibitor.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating cancer of the present disclosure can provide any amount or any level of treatment. Furthermore, the treatment provided by the methods of the present disclosure can include treatment of one or more conditions or symptoms or signs of the cancer being treated. Also, the treatment provided by the methods of the present disclosure can encompass slowing the progression of the cancer. For example, the methods can treat cancer by virtue of enhancing the T cell activity or an immune response against the cancer, reducing tumor or cancer growth or tumor burden, reducing metastasis of tumor cells, increasing cell death of tumor or cancer cells or increasing tumor regression, and the like. In accordance with the foregoing, provided herein are methods of reducing tumor growth or tumor burden or increasing tumor regression in a subject. In exemplary embodiments, the method comprises administering to the subject a RANK pathway inhibitor optionally in combination with a CDK inhibitor, e.g., a CDK4/6 inhibitor. In exemplary embodiments, the subject is or has been treated with a CDK inhibitor, e.g., a CDK4/6 inhibitor, and the method comprises administering to the subject a RANK pathway inhibitor.

In various aspects, the methods treat by way of delaying the onset or recurrence of the cancer by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject. In exemplary aspects, the methods of the present disclosure provide treatment by way of delaying the occurrence or onset of metastasis. In various instances, the methods provide treatment by way of delaying the occurrence or onset of a new metastasis. Accordingly, provided herein are methods of delaying the occurrence or onset of metastasis in a subject with cancer. In exemplary embodiments, the method comprises administering a RANK pathway inhibitor to the subject optionally in combination with a CDK inhibitor, e.g., a CDK4/6 inhibitor.

In exemplary instances, the treatment provided may be described in terms of or supported by data obtained from a clinical trial wherein the endpoints of the trial are progression-free survival (PFS), overall survival (OS), or time to deterioration of Eastern Cooperative Oncology Group (ECOG) performance status. In various aspects, the present disclosure provides a method of increasing PFS, OS, or time to deterioration of ECOG performance status in a subject with a cancer. In exemplary embodiments, the cancer is resistant to or with a reduced sensitivity to a CDK inhibitor, e.g., CDK4/6 inhibitor, and the method comprises administering to the subject a RANK pathway inhibitor optionally in combination with a CDK inhibitor, e.g., CDK4/6 inhibitor. As used herein, the term “progression-free survival” or “PFS” means the time a treated patient experiences without cancer getting worse (by whatever measure is being used to measure worsening). The term “overall survival” means how long the patient lives after treatment. ECOG performance status is a grade or score according to a scale used by doctors and researchers to assess a patient's disease, e.g., how the disease is progressing/regressing, how the disease affects the daily living abilities of the patient, and determine appropriate treatment and prognosis. ECOG performance status is determined according to the following criteria:

| Score | ECOG |
|---|---|
| 0 | Fully active, able to carry on all pre-disease performance |
| | without restriction |
| 1 | Restricted in physically strenuous activity but ambulatory and |
| | able to carry out work of a light or sedentary nature, e.g., light |
| | house work, office work |
| 2 | Ambulatory and capable of all selfcare but unable to carry out any |
| | work activities. Up and about more than 50% of waking hours |
| 3 | Capable of only limited selfcare, confined to bed or chair more |
| | than 50% of waking hours |
| 4 | Completely disabled. Cannot carry on any selfcare. Totally |
| | confined to bed or chair |
| 5 | Dead |

Oken et al., Am. J. Clin. Oncol 5: 649-655 (1982)

In exemplary aspects, the treatment provided may be by way of reducing the level of circulating tumor cells (CTCs). Accordingly, the present disclosure provides methods of reducing the level of CTCs in a subject. In exemplary embodiments, the method comprises administering to the subject a RANK pathway inhibitor optionally in combination with a CDK4/6 inhibitor. In exemplary embodiments, and the method comprises administering a RANK pathway inhibitor to the subject.

Routes and Timing of Administration

The RANK pathway inhibitor and/or the CDK inhibitor, e.g., CDK4/6 inhibitor, of the presently disclosed pharmaceutical composition or of the presently disclosed methods can be administered to the subject via any suitable route of administration. For example, the active agent can be administered to a subject via parenteral, nasal, oral, pulmonary, topical, vaginal, or rectal administration. The following discussion on routes of administration is merely provided to illustrate exemplary embodiments and should not be construed as limiting the scope in any way.

In exemplary aspects, the RANK pathway inhibitor and/or the CDK inhibitor, e.g., CDK inhibitor, of the presently disclosed pharmaceutical composition or of the presently disclosed methods is formulated for parenteral administration. The term, “parenteral” means not through the alimentary canal but by some other route such as subcutaneous, intramuscular, intraspinal, or intravenous. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The active agent (the RANK pathway inhibitor and/or the CDK inhibitor) can be administered with a physiologically acceptable diluent in a pharmaceutical carrier, such as a sterile liquid or mixture of liquids, including water, saline, aqueous dextrose and related sugar solutions, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. In exemplary aspects, the formulation for parenteral administration includes a soap. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof. In exemplary instances, preservatives and buffers are present in the parenteral formulation. In order to minimize or eliminate irritation at the site of injection, such compositions can contain one or more nonionic surfactants having a hydrophile-lipophile balance (HLB) of from about 12 to about 17. The quantity of surfactant in such formulations typically ranges from about 5% to about 15% by weight. Suitable surfactants include polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations in some aspects are presented in unit-dose or multi-dose sealed containers, such as ampoules and vials, syringes, and can be stored in a freeze-dried (lyophilized) condition requiring only the addition of the sterile liquid excipient, for example, water, for injections, immediately prior to use. Extemporaneous injection solutions and suspensions in some aspects are prepared from sterile powders, granules, and tablets of the kind previously described.

In exemplary aspects, the RANK pathway inhibitor and/or the CDK inhibitor are formulated for injection. Injectable formulations are in accordance with the present disclosure. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)).

Optionally, the RANK pathway inhibitor is administered to the subject via subcutaneous injection.

In various instances the CDK inhibitor is administered orally to the subject. Formulations suitable for oral administration can consist of (a) liquid solutions, such as an effective amount of the analog of the present disclosure dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the analog of the present disclosure in a flavor, usually sucrose and acacia or tragacanth, as well as pastilles comprising the analog of the present disclosure in an inert base, such as gelatin and glycerin, or sucrose and acacia, emulsions, gels, and the like containing, in addition to, such excipients as are known in the art.

With regard to the RANK pathway inhibitor and/or the CDK inhibitor, each may be administered according to any regimen including, for example, daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In exemplary aspects of the presently disclosed methods, when both a RANK pathway inhibitor and a CDK inhibitor are administered, the RANK pathway inhibitor and the CDK inhibitor are administered separately. In exemplary aspects, the RANK pathway inhibitor is administered to the subject once every 2 to 6 weeks (once every 2 weeks, once every 3 weeks, once every 4 weeks, once every 5 weeks, once every 6 weeks). Optionally, the RANK pathway inhibitor is administered to the subject every 4 weeks. Optionally, the RANK pathway inhibitor is administered to the subject once every 2 to 8 months (once every 2 months, once every 3 months, once every 4 months, once every 5 months, once every 6 months, once every 7 months, once every 8 months). In various instances, the CDK inhibitor is administered once daily to the subject. Optionally, the RANK pathway inhibitor is administered to the subject via injection, optionally, intramuscular injection or subcutaneous injection. In various instances the CDK inhibitor is administered orally to the subject.

In alternative aspects, the RANK pathway inhibitor and the CDK inhibitor are simultaneously administered to the subject. In exemplary aspects, the composition can be administered as separate formulations on in opine formulation.

Dosages

The active agents (the RANK pathway inhibitor and/or the CDK inhibitor) are believed to be useful in methods of increasing or restoring responsiveness of a cancer cell or tumor to treatment with a CDK inhibitor, delaying the occurrence or onset of a metastasis, reducing tumor growth or tumor burden or increasing tumor regression, increasing progression-free survival, overall survival, or time to deterioration of Eastern Cooperative Oncology Group (ECOG) performance status, and reducing the level of circulating tumor cells (CTCs) in a subject, as described herein, and are thus believed to be useful in methods of treating or preventing one or more diseases, e.g., cancer. For purposes of the disclosure, the amount or dose of the active agent (the RANK pathway inhibitor and/or the CDK inhibitor) administered should be sufficient to effect, e.g., a therapeutic or prophylactic response, in the subject or animal over a reasonable time frame. For example, the dose of the active agent (the RANK pathway inhibitor and/or the CDK inhibitor) should be sufficient to treat cancer as described herein in a period of from about 1 to 4 about days or about 1 to about 4 weeks or longer, e.g., about 5 to about 20 or more weeks, from the time of administration. In certain embodiments, the time period could be even longer. The dose will be determined by the efficacy of the particular active agent and the condition of the animal (e.g., human), as well as the body weight of the animal (e.g., human) to be treated.

Many assays for determining an administered dose are known in the art. For purposes herein, an assay, which comprises comparing tumor weight, the total flux emission, which represents tumor burden, or percentage (e.g., relative percentage) of Ki67-positive cells or percentage of p-pRb-positive cells, which provides a proliferation index, upon administration of a given dose of the active agent to a mammal among a set of mammals, each set of which is given a different dose, could be used to determine a starting dose to be administered to a mammal in a clinical trial. Methods of measuring tumor weight, total flux, or percentage (e.g., relative percentage) of Ki67-positive cells or percentage of p-pRb-positive cells are known in the art and described herein.

The dose of the active agent (the RANK pathway inhibitor and/or the CDK inhibitor) also will be determined by the existence, nature and extent of any adverse side effects that might accompany the administration of a particular active agent. Typically, the attending physician will decide the dosage of the active agent with which to treat each individual patient, taking into consideration a variety of factors, such as age, body weight, general health, diet, sex, active agent to be administered, route of administration, and the severity of the condition being treated. By way of example and not intending to limit the present disclosure, the dose of the active agent of the present disclosure can be about 0.0001 to about 1 g/kg body weight of the subject being treated/day, from about 0.0001 to about 0.001 g/kg body weight/day, or about 0.01 mg to about 1 g/kg body weight/day.

Controlled Release Formulations

In some embodiments, the active agents (the RANK pathway inhibitor and/or the CDK inhibitor) described herein can be modified into a depot form, such that the manner in which the active agent is released into the body to which it is administered is controlled with respect to time and location within the body (see, for example, U.S. Pat. No. 4,450,150). Depot forms of active agents (the RANK pathway inhibitor and/or the CDK inhibitor) can be, for example, an implantable composition comprising the active agents and a porous or non-porous material, such as a polymer, wherein the active agent is encapsulated by or diffused throughout the material and/or degradation of the non-porous material. The depot is then implanted into the desired location within the body of the subject and the active agent is released from the implant at a predetermined rate.

The pharmaceutical composition comprising the active agent in certain aspects is modified to have any type of in vivo release profile. In some aspects, the pharmaceutical composition is an immediate release, controlled release, sustained release, extended release, delayed release, or bi-phasic release formulation. Methods of formulating peptides for controlled release are known in the art. See, for example, Qian et al., J Pharm 374: 46-52 (2009) and International Patent Application Publication Nos. WO 2008/130158, WO2004/033036; WO2000/032218; and WO 1999/040942.

The instant compositions can further comprise, for example, micelles or liposomes, or some other encapsulated form, or can be administered in an extended release form to provide a prolonged storage and/or delivery effect.

Subjects

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. Optionally, the human is a female aged 18 years or more. In some aspects, the human is female. In various instances, the subject is a pre/perimenopausal or postmenopausal woman.

In exemplary aspects, the subject has cancer or a tumor. The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In particular embodiments, the tumor is non-small cell lung cancer (NSCLC), head and neck cancer, renal cancer, triple negative breast cancer, or gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject.

In exemplary aspects, the subject has cancer with a metastasis, an unresectable tumor, or a combination thereof. In various aspects, the cancer or tumor exhibits or has exhibited a resistance or reduced sensitivity to treatment with a CDK inhibitor. In exemplary aspects, the subject has breast cancer, optionally, luminal breast cancer or triple negative breast cancer. In various instances, the breast cancer hormone receptor (HR)-positive and/or HER2-negative. In various aspects, the breast cancer is advanced breast cancer and/or metastatic breast cancer. In various aspects, the subject has HR+/HER2− advanced or metastatic breast cancer that has progressed after taking endocrine therapy. In some aspects, the subject is a hormone receptor-positive (HR+)/HER2-negative (HER2−) advanced or metastatic breast cancer patient previously treated with endocrine therapy and chemotherapy after cancer has spread/metastasized. In various instances, the subject has HR+/HER2− advanced or metastatic breast cancer that has not been treated with hormonal therapy before in postmenopausal women (Arimidex (chemical name: anastrozole), Aromasin (chemical name: exemestane), and Femara (chemical name: letrozole). In various instances, the subject has HR+/HER2− advanced or metastatic breast cancer that has grown after being treated with hormonal therapy in postmenopausal women. In certain aspects, the subject is a pre/perimenopausal or postmenopausal woman with HR+, human epidermal growth factor receptor 2 (HER2)-negative advanced or metastatic breast cancer, as initial endocrine-based therapy. Optionally, the subject is a postmenopausal woman with HR+, HER2− advanced or metastatic breast cancer, as initial endocrine-based therapy or following disease progression on endocrine therapy. In exemplary aspects, the hormone receptor is an estrogen receptor (ER). In various aspects, the subject has ER+/HER2− advanced or metastatic breast cancer that has progressed after taking endocrine therapy. In some aspects, the subject is a estrogen receptor-positive (ER+)/HER2-negative (HER2−) advanced or metastatic breast cancer patient previously treated with endocrine therapy and chemotherapy after cancer has spread/metastasized.

In exemplary aspects, the subject has a cancer or tumor, as described in Knudson and Witkiewicz, 2017, supra. In exemplary aspects, the subject has Mantle Cell Lymphoma, Acute Lymphoblastic Lymphoma, Multiple Myeloma, Acute Myeloid Leukemia, Chronic Myelogenous Leukemia, Medulloblastoma, Liposarcoma, Rhabdomyosarcoma, Ewing Sarcoma, Synovial Sarcoma, Rhabdoid Tumor, MPNST, Gastric Cancer, Pancreatic Neuroendocrine cancer, Pancreatic Ductal Adenocarcinoma, Non small-cell lung cancer, Squamous Cell Carcinoma of Head and Neck, Glioma, Melanoma, Ovarian Cancer, Breast cancer, bladder cancer, renal cancer, colon cancer, esophageal cancer, or prostate cancer.

In various instances, the cancer comprises cells that express RANK or RANK-L. In various instances, the cancer comprises cells that over-express RANK or RANK-L. In exemplary embodiments, cells of the cancer overexpress one or more of RANK, CDK 4, CDK 6, or Cyclin D. In exemplary aspects, the subject has an increased level of circulating tumor cells (CTCs). In exemplary aspects, cells of the cancer overexpress one or more of RANK, CDK 4, CDK 6, or Cyclin D and/or the subject has an increased level of CTCs. In exemplary instances, the method comprises administering to the subject a RANK pathway inhibitor optionally in combination with the CDK inhibitor. In exemplary embodiments, the subject is or has been treated with a CDK inhibitor and (i) cells of the cancer overexpress one or more of RANK, CDK 4, CDK 6, or Cyclin D, (ii) the subject has an increased level of circulating tumor cells (CTCs), or (iii) a combination thereof, and the method comprises administering a RANK pathway inhibitor to the subject. In some aspects, the subject has been treated with endocrine therapy. In some aspects, the subject exhibited disease progression on endocrine therapy. In various aspects, the subject has previously been treated with endocrine therapy and chemotherapy after cancer has spread/metastasized. In some aspects, the subject has advanced or metastatic breast cancer that has progressed after taking endocrine therapy.

In exemplary aspects, the cancer or tumor cell is positive for one or more markers of intrinsic resistance as described in Knudson and Witkiewicz, 2017, supra. In exemplary aspects, the cancer or tumor demonstrates a loss of RB, high expression of p16INK4a, cyclin E overexpression or amplification of Cyclin E1/E2, E2Foverexpression of E2F3 amplification, Cyclin D1 amplification or translocation, CDK4 amplification, or CDKN2A loss. In exemplary aspects, the cancer or tumor cell is positive for one or more putative markers of resistance and sensitivity, as described in Knudson and Witkiewicz, 2017, supra.

The following examples are given merely to illustrate the present invention and not in any way to limit its scope.

### EXAMPLES

### Example 1

This example describes the materials and methods used in the studies described herein unless noted otherwise.

Cell Culture

All cell lines were initially obtained from ATCC. Human breast carcinoma cell lines MDA-MB-231GFP+Luc+ and MCF-7GFP+Luc+ (herein designated by MDA-MB-231 and MCF-7) were provided by Sérgio Dias Lab (IMM), and derived from parental cells by lentiviral transduction with GFP-Luciferase lentiviral particles and cell sorting of pure GFP+ cell populations. MDA-MB-436 and MDA-MB-468 cells were provided by Sérgio Dias Lab (IMM). T47D cells were provided by Philippe Clézardin Lab at INSERM. MDA-MB-231, MDA-MB-436, and MDA-MB-468 cells were cultured in Dulbecco's Modified Eagle's Medium (DMEM, Gibco) supplemented with 10% (v/v) fetal bovine serum (FBS, Gibco), 1% (v/v) Penicillin/Streptomycin (10,000 U/mL Penicillin, 10,000 μg/mL Streptomycin, Gibco). MCF-7 and T47D cells were cultured in the same medium, additionally supplemented with 0.01 mg/mL insulin (Gibco). Cells were maintained at 37° C. with 5% CO2.

RANK (TNFRSF11A) Overexpression

For lentiviral transduction, MCF-7 and T47D cells were seeded in 6-well-plates, at a density of 2×10⁵ cells/well. 24 hours after seeding, medium was replaced by fresh medium containing RANK lentiviral overexpression particles (RANK (TNFRSF11A) overexpression plasmid pReceiver-Lv121 (EX-00007-Lv121), GeneCopoeia) at different multiplicity of infection (MOI). Cells were selected with with 0.5 μg/mL (MCF-7) or 1.5 μg/ml (T47D) puromycin dihydrochloride (Sigma-Aldrich), starting three days after transduction. RANK overexpression (RANK OE) was confirmed by RT-qPCR.

RANK (TNFRSF11A) Knockdown

For lentiviral transduction, MDA-MB-231GFP+Luc+ cells were seeded in 24-well plates, at a density of 4×10⁴ cells/well. 24 hours after seeding, medium was replaced by fresh medium containing 8 μg/ml Polybrene (Sigma) and RANK shRNA (h) lentiviral particles (15-20 μl/well; sc-42960-V, Santa Cruz) or control shRNA lentiviral particles (15-20 μl/well; sc-108080, Santa Cruz). Cells were selected with 0.5 μg/mL Puromycin dihydrochloride (Sigma) starting three days after transduction. RANK overexpression KD was confirmed by RT-qPCR.

RT-qPCR

For RT-qPCR analysis of mRNA expression, cells were grown up to 80-90% confluency and total RNA was extracted using the NZY Total RNA Isolation kit (Nzytech), according to manufacturer's instructions. Total RNA was quantified in a NanoDrop spectrophotometer (Thermo Ficsher Scientific). Total RNA (500 ng to 1 μg) was treated with RQ1 RNase-free DNase I (Promega) for 30 min at 37° C., according to manufacturer's instructions. DNase I-treated RNA was reverse transcribed using the NZY M-MuLV First-Strand cDNA Synthesis kit (Nzytech), and Oligo(dT)20 primer, according to manufacturer's instructions.

cDNAs were amplified by real-time PCR using TaqMan Gene Expression Master Mix (Applied Biosystems), according to manufacturer's instructions, and specific primers for human RANK (PPH01102C, SA Biosciences) and GAPDH (PPH00150F, SA Biosciences). Gene expression was normalized using the housekeeping gene GAPDH, and relative mRNA expression was calculated using the 2-ΔΔCt method.

Western Blot

Activation of RANK pathway upon stimuli with RANKL was analysed by Western blot.

For this purpose, 4×105 cells were seeded in 6-well plates for 24 h, and serum-starved in low-serum medium (0,1% FBS, 1% Pen/Step) for another 24 h. Medium was replaced by fresh low-serum medium containing 1 μg/mL human RANKL (Amgen) and total cell lysates obtained at different time points. Total cell lysates were prepared with RIPA buffer containing protease and phosphatase inhibitors cocktails (1:100; Santa Cruz), according to manufacturer's instructions. Total protein was quantified using Pierce BCA Protein Assay Kit (ThermoSicentific), according to manufacturer's instructions. Proteins were resolved by SDS-PAGE, using 10% polyacrylamide gels, and then transferred to nitrocellulose membranes using an iBlot®2 Gel Transfer Device (Invitrogen), according to manufacturer's instruction.

Membranes were blocked for 1 h at room temperature (RT) in 5% Non-Fat Dry Milk (NFDM) in PBS-0.1% Tween (PBST) for β-actin; or in 5% bovine serum albumin (BSA) (Santa Cruz) for other antibodies. Membranes were incubated with the following specific antibodies, overnight at 4° C.: mouse anti-β Actin antibody (Ab6276; Abcam), rabbit polyclonal to NFkB p65 (ab16502, Abcam), rabbit monoclonal anti NF-kB p65 (D14E12) (#8242, Cell Signaling), rabbit monoclonal anti Phospho-NF-kB p65 (Ser536) (93H1) (#3033, Cell Signaling), mouse monoclonal anti IkBα (L35A5) (#4814, Cell Signaling), rabbit monoclonal anti Phospho-IkBα (Ser32) (14D4) (#2859, Cell Signaling), rabbit monoclonal anti Vimentin (D21H3) (#5471, Cell Signaling), rabbit monoclonal anti N-Cadherin (D4R1H) (#13116, Cell Signaling), rabbit monoclonal anti β-Catenin (D10A8) (#8480, Cell Signaling), rabbit monoclonal anti Snail (C15D3) (#3879, Cell Signaling), rabbit monoclonal anti Slug (C19G7) (#9585, Cell Signaling), rabbit monoclonal anti E-Cadherin (24E10) (#3195, Cell Signaling), mouse monoclonal anti Nanog (hNanog.2) (145768-80, eBioscience), mouse monoclonal anti Sox2 (245610) (MAB2018, R&D systems), mouse monoclonal anti OCT4 (7F9.2) (MAB4419, Millipore), rabbit monoclonal anti Phospho-Rb (Ser807/811) (D20B12) (#8516, Cell Signaling), rabbit monoclonal anti Cyclin D1 (92G2) (#2978, Cell Signaling), rabbit monoclonal anti Cyclin E (HE12) (05-363, Sigma), rabbit monoclonal anti CDK2 (78B2) (#2546, Cell Signaling), rabbit monoclonal anti CDK4 (D9G3E) (#12790, Cell Signaling), rabbit monoclonal anti CDK6 (DCS83) (#3136, Cell Signaling). After washing with PBST, membranes were incubated with horseradish peroxidase-conjugated (HRP) specific secondary antibodies: anti-mouse-HRP IgG and anti-rabbit-HRP IgG (1:5000; Cell Signaling), for 2 h at RT. Proteins were detected using a Novex® ECL HRP chemiluminescent substrate reagent kit (Invitrogen) according to the manufacturer's instructions, and x-ray films (Fujifilm) developed in a Curix 60 processor (AGFA), or the Amersham™ Imager 680 (GE Healthcare Life Sciences). Normalized protein expression was calculated using FIJI software and band densitometry analysis.

Flow Cytometry

For RANK expression analysis, trypsinized cells were washed and incubated with specific mouse antibody against human RANK (Amgen Inc.) for 30 min at 4° C. in the dark, then washed and labelled with goat anti-mouse secondary antibody conjugated with cy5IgM (1:100; Dianova) for 30 min at 4° C., in the dark. After staining, labelled cells were centrifuged at 120 g, washed, ressuspended in 1×PBS with 0.1% FBS and acquired on a LSR Fortessa (BD Biosciences, California, USA). Data was analyzed with FlowJo software.

Tumorsphere Formation Assay

Adherent non-confluent cells were harvested, washed in 3D Tumorsphere Medium XF (PromoCell) and seeded in ultra-low attachment 6-well plates (Corning Inc.) at a density of 10,000 cells/ml in the same medium, in triplicate. After 7 days, average tumorsphere size was calculated by measuring all tumorspheres >50 μm in diameter per well. Sphere Forming Capacity (SFC) (%) was determined as the number of mammospheres >50 μm/number of cells seeded)×100.

Viability Assays

Cells were seeded in 96 well-plates (2-5×10⁴ cells/mL), with or without paclitaxel (Y0000698, Sigma), doxorubicin hydrochloride (D2975000, Sigma), palbociclib (PD 0332991 isethionate, PZ0199, Sigma), ribociclib (LEE011 succinate, SC-488174, Santa Cruz), abemaciclib mesylate (LY2835219, S7158, Selleckchem), fulvestrant (S1191, Selleckchem), everolimus (#73122, StemCell Technologies), full length human OPG (SRP3132, Sigma), and OPG-Fc (Amgen). Medium was replaced every 2 days. After 7 days, 1:10 Alamar blue (Invitrogen) was added to each well and fluorescence was measured 2 hours after incubation (excitation 560 nm; emission 590 nm) in an Infinite M200 microplate reader (Tecan).

Animal Models

All animal experiments were reviewed and approved by Institutional Animal Welfare Body, and licensed by the national entity Direcção Geral de Alimentagçãe Veterinaria (DGAV). In all studies involving animals, mice were handled and euthanized in accordance with approved institutional, national and international guidelines, applying the Principle of the 3Rs.

Orthotopic Xenograft Models

For the orthotopic MCF-7 xenografts' model, four week old NOD scid gamma (NSG) mice (Charles River) were supplemented with subcutaneous 17β-estradiol pellets (60-day release, 0.36 mg/pellet, Innovative Research of America), six days prior to breast cancer cell injection (day −6). Pellets were inserted with a trochar in the mid-scapular region of the mouse under mild isofluorane anesthesia.

Cells were harvested at the exponential phase of growth and resuspended at 2×10⁵ cells/ml (MCF-7 xenografts) or 1.0×10⁶ cells/ml (MDA-MB-231 xenografts) in 50% phenol-free matrigel solution (Corning). Mice were injected unilaterally with 100 μl of cell suspension, directly into the second thoracic fat pad by subcutaneous injection at the base of the nipple.

Tumor growth was monitored weekly by luminescence analysis. Mice were injected with 100 μl/10 g body weight (BW) XenoLight D-Luciferin—K+ Salt Bioluminescent Substrate (PerkinElmer) and after 4 min they were anesthetized with 75 mg/Kg BW Ketamine+1 mg/Kg BW Medetomidine. After approximately 6 min luminescence was analysed in an IVIS Lumina, using Living Image 3.0 software (30 s of exposure; field of view D 12.5 cm; subject height 1.5 cm), and mice recovered with 1 mg/Kg BW Atipamezole.

For MCF-7 drug sensitivity experiments mice were randomized based on tumor size measured by bioluminescence and treated with Palbociclib 25 mg/Kg/day p.o. (PD 0332991 isethionate, PZ0199, Sigma) plus Fulvestrant 1 mg/day s.c. (S1191, Selleckchem) or vehicle (0,1M Na Lactate (L7900, Sigma) plus 95% corn oil (C8267, Sgma), 5% DMSO (Sigma)).

For MDA-MB-231 drug sensitivity experiments Balb/c nude mice were randomized based on tumor size measured by bioluminescence (n=2/group) and treated with palbociclib 25 mg/Kg/d p.o.; OPG-Fc 10 mg/kg i.p. twice weekly; or Palbociclib+OPG-Fc 10 mg/Kg.

Mice were sacrificed by administration of 0.25 mg/Kg BW Sodium Pentobarbital (Eutasil). At necropsy, primary tumors were harvested and sectioned into two fragments for paraffin embedding or snapshot freezing. Organs were harvested and paraffin embedded.

Experimental Metastases Model

Nod scid gama (NSG) mice were inoculated in the tail vein with 2.5×10⁶ cells/ml, MCF-7GFP+Luc+ (Parental) or MCF-7 RANK OE GFP+Luc+ (RANK OE) cells (n=3/group). Tumours were imaged by bioluminescence 2 h and every week post tumour inoculation till the end of the experience.

For CTC analysis, venous blood was collected by cardiac puncture before sacrifice, into 1.5 mL centrifuge tubes with 5 μl EDTA 0,5M (pH8.0). Erythrocytes were lysed by incubation with 1×RBC Lysis Buffer Multi-species (eBioscience) for 13 min at RT. Cells were washed with FACS buffer and centrifuged for 3 min at 2,000 rpm. The supernatant was discarded and cells were ressuspended in FACS buffer. Samples were analysed for GFP expression in a BD Fortessa 2 flow cytometer.

Immunohistochemistry

5 μm tissue sections from FFPE samples were stained by immunohistochemistry (IHC) for the detection of Ki67 and p-pRb (Ser807/11). Deparaffinization and antigen retrieval was performed in a PT Link Pre-Treatment Module for Tissue Specimens (Dako), using Antigen Retrieval Solution pH9.0, at 94° C. for 20 min. Endogenous peroxidase was blocked with Preoxidase Blocking Solution (Dako) for 10 min at RT, and total protein was blocked by incubation with Protein Block Solution (Dako), for 20 min at RT. Slides were incubated for 30 min with rabbit anti-human Ki67 primary antibody (1:100, MIB-1, Dako) or rabbit monoclonal anti Phospho-Rb (Ser807/811) (D20B12) (1:400, #8516, Cell Signaling), in Antibody Diluent (Dako). Slides were incubated with EnVision™ Detection System, rabbit/mouse (Dako), according to manufacturer's instructions, followed by 5 min of incubation with DAB (Dako). Slides were counterstained with hematoxylin, dehydrated, mounted with Quick-D mounting medium (Klinipath) and visualized in a bright field microscope (LeicaDM750 with a Leica ICC50 HD camera). Imunoratio was obtained through the calculation of the percentage of DAB-stained nuclear area over total nuclear area (hematoxylin-stained nuclei regions) (5 fields, 400×, ImageJ software).

Statistical Analysis

Data was analyzed using GraphPad Prism6 software. The number of replicates performed for each experiment is indicated. Statistics were performed by one-way ANOVA or unpaired t-test (in vitro experiments); two-way ANOVA (tumour burden and mice BW). Results are presented as mean with SEM and p-value <0.05 was considered significant. Survival analyzes used the Kaplan-Meier method and log-rank test, and Cutoff finder software (Budczies et al. (2012), PLoS ONE 7(12): e51862.).

### Example 2

This example shows the generation and characterization of RANK over-expressing cancer cells.

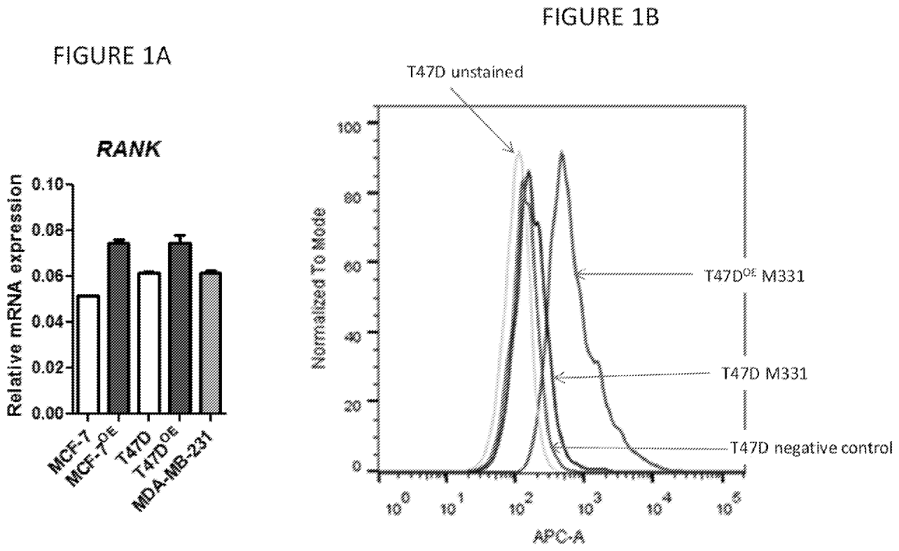

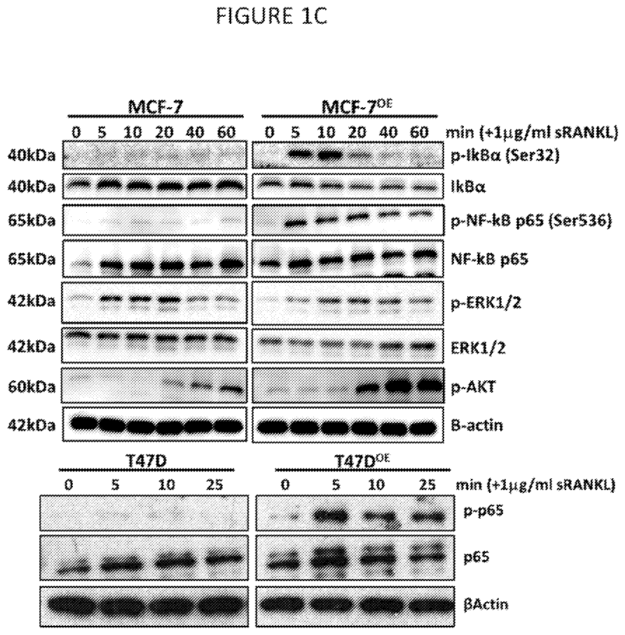

LBC cells normally express low levels of RANK. To test the effect of RANK expression levels in this context, parental cell lines were used to generate RANK overexpressing (OE) cells by lentiviral transduction. Briefly, each of MCF-7 and T47D cells was seeded in multi-well-plates and then exposed to fresh medium containing RANK lentiviral overexpression particles. Cells were selected with puromycin dihydrochloride beginning three days post-transduction. Over-expression of RANK was confirmed by RT-qPCR (FIG. 1A) and flow cytometry (FIG. 1B).

As shown in FIG. 1A, both MCF-7 OE cells and T47D OE cells exhibited increased expression of RANK compared to their parental counterparts. Also, as shown in FIG. 1B, T47D OE cells demonstrated higher RANK expression as determined by flow cytometry in comparison to the parental counterpart.

The downstream signal transduction of RANK OE cells were next tested by stimulating the MCF-7 OE cells and T47D OE cells with soluble RANK Ligand (sRANKL) then analyzing by Western blotting phosphorylation of IkBα, NFkB, ERK and AKT. Degradation of IkBα also was determined for this purpose. As shown in FIG. 1C, the stimulated MCF-7 OE cells and T47D OE cells exhibited increased RANK pathway activity.

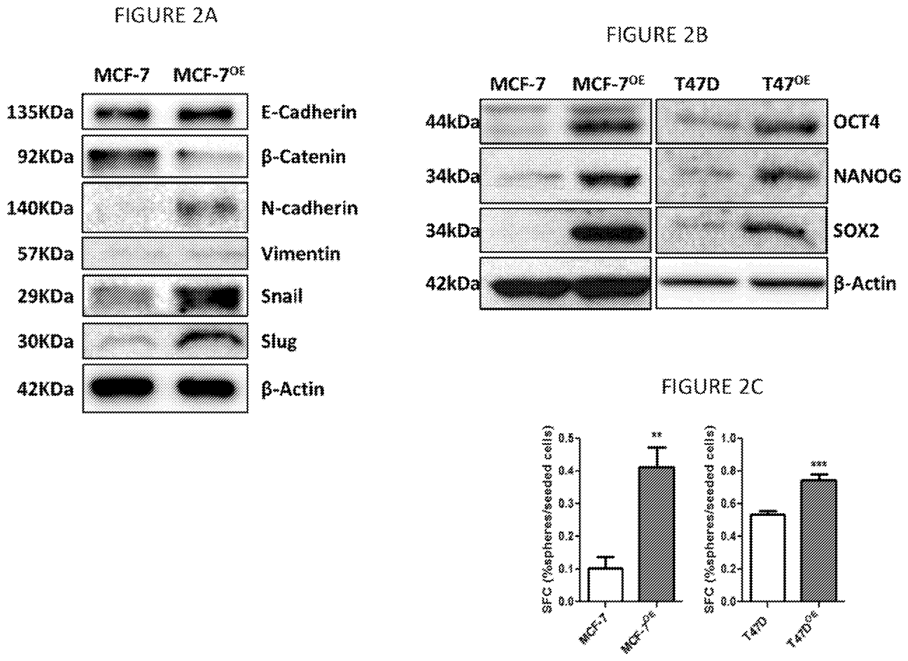

The phenotype of the RANK OE cells were tested by analyzing expression levels of epithelial marker (including β-catenin and E-cadherin), mesenchymal markers (including N-cadherin, vimentin, Snail and Slug), and stem cell markers (including SOX2, Oct4 and NANOG). As shown in FIG. 2A, MCF-7 OE cells demonstrated decreased expression of β-catenin and increased expression of the mesenchymal markers. FIG. 2B shows the stem cell marker expression levels of MCF-7 OE cells and T47D OE cells, and their parental counterparts. As shown in FIG. 2B, the OE cells overexpressed the stem cell markers SOX2, OCT4, and NANOG, thereby demonstrating stem cell-like features.

RANK OE cell phenotype was also characterized by analyzing the sphere forming capacity (SFC) of the RANK OE cells. Briefly, adherent non-confluent cells were harvested, washed and seeded in ultra-low attachment multi-well and average tumorsphere size was calculated by measuring all tumorspheres >50 μm in diameter per well. Sphere Forming Capacity (SFC) (%) was determined as the number of mammospheres >50 μm/number of cells seeded)×100. The data from this assay is plotted in the graph of FIG. 2C. As shown here, the % SFC of RANK OE cells (both MCF-7 OE and T47D OE cells) were greater than their parental counterparts which express RANK at low levels.

This example demonstrated the generation of RANK OE LBC cells and the phenotypic differences of these cells in comparison to their parental counterparts.

### Example 3

This example demonstrates RANK overexpression correlates with reduced sensitivity to CDK4/6 inhibitors and blockade of the RANK pathway sensitizes Luminal Breast Cancer (LBC) and Triple Negative Breast Cancer (TNBC) cells to CDK4/6 inhibitors.

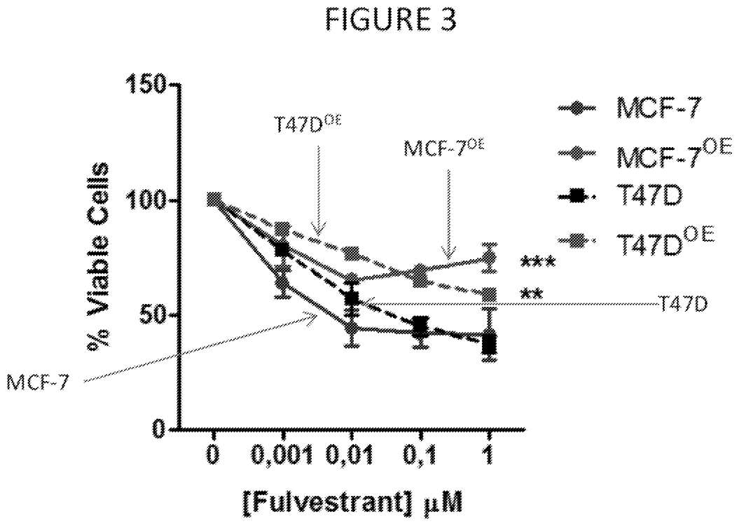

As shown in Example 2, the RANK OE LBC cells exhibited an altered phenotype. We next tested if RANK OE cells also differed from their parental counterparts by way of their response to targeted therapies commonly used to treat LBC. In particular, RANK OE cells were tested for responsiveness to hormone therapy. Briefly, RANK OE cells were seeded in multi-well plates and exposed to fulvestrant for 7 days. Cell viability was assessed by an Alamar blue assay and the results are shown in FIG. 3. As shown in this figure, RANK OE cells demonstrated decreased sensitivity to fulvestrant.

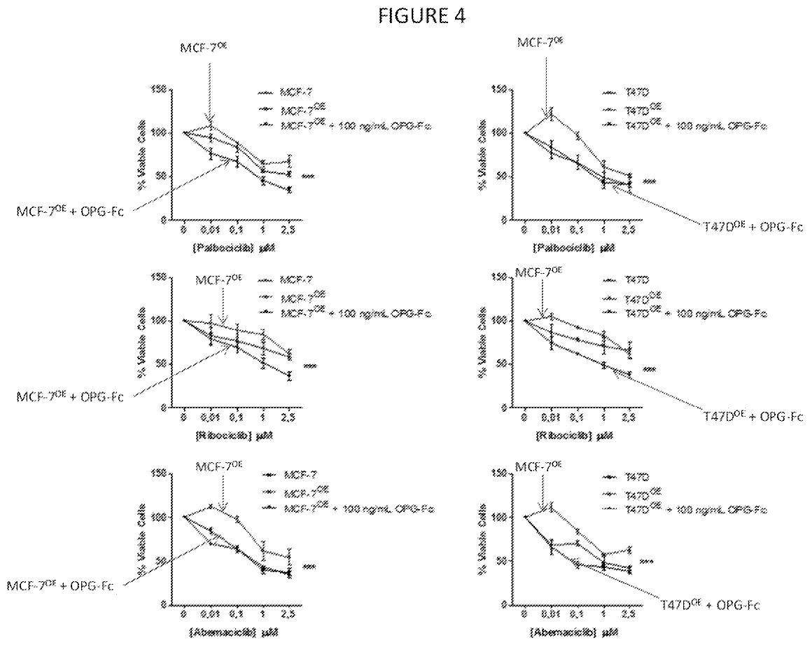

A similar drug sensitivity assay was used to test the sensitivity of RANK OE cells to CDK4/6 inhibitors. In this assay, however, RANK OE cells were exposed to a CDK 4/6 inhibitor alone or in combination with an inhibitor of the RANK pathway, in order to test the effect of blocking the RANK pathway in this context. Briefly, RANK OE cells were seeded in multi-well plates and exposed to palbociclib, ribociclib or abemaciclib alone or in combination with OPG-Fc for 7 days. Osteoprotegrin (OPG) is the physiologic soluble decoy receptor for RANK ligand (RANKL), and OPG-Fc is a fusion protein comprising only the four cysteine-rich domains of OPG (D1 to D4) fused with the Fc domain of an IgG1 antibody. Cell viability was assessed by an Alamar blue assay and the results are shown in FIG. 4. As shown in FIG. 4, RANK OE cells demonstrated a decreased sensitivity to each of the CDK4/6 inhibitors tested (red lines). Interestingly, when the RANK OE cells were exposed to the CDK4/6 inhibitor in combination with the inhibitor of the RANK pathway (OPG-Fc), the sensitivity to the CDK4/6 inhibitor was restored back to a level that is similar to that demonstrated by the parental counterpart cells which express RANK at low levels. These results suggest that sensitivity to CDK4/6 inhibitors may be restored by using CDK4/6 inhibitors in combination with an inhibitor of the RANK pathway.

### Example 4

This example demonstrates that elevated RANK expression levels correlate with resistance to CDK4/6 combination therapy and that the inhibition of the RANK pathway restores sensitivity to CDK4/6 combination therapy.

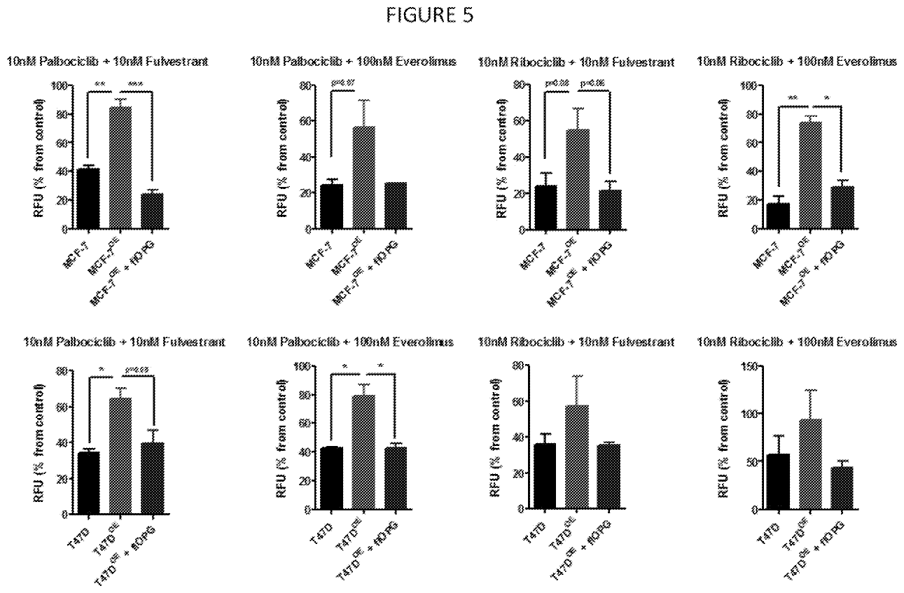

The data above shows that elevated RANK expression levels are related with resistance to therapy and suggests that the inhibition of the RANK pathway restores or induces sensitivity to chemo treatments. These concepts were further supported by testing the sensitivity of RANK OE cells to CDK4/6 inhibitor combination therapy alone or in further combination with an inhibitor of the RANK pathway. In this study, the CDK4/6 inhibitors palbociclib and ribociclib were exposed to RANK OE cells in combination with fulvestrant or everolimus. This combination therapy was then tested alone or in combination with OPG, an inhibitor of the RANK pathway. In this assay, the full-length OPG protein was used, in contrast to the previous example utilizing OPG-Fc. The assay was carried out as essentially described above in Examples 1-3 and the results are shown in FIG. 5.

As shown in FIG. 5, RANK OE cells displayed a decreased sensitivity to CDK4/6 inhibitor combination therapy (palbociclib+fulvestrant; palbociclib+everolimus; ribociblib+fulvestrant; ribociblib+everolimus (red bars)) as shown by increased cell viability relative to the parental counterparts (black bars). When CDK4/6 inhibitor combination therapy was further combined with an inhibitor of the RANK pathway (OPG), the sensitivity to the CDK4/6 inhibitor combination therapy was restored back to levels of the parental counterparts.

### Example 5

This example demonstrates that the RANK pathway contributes to the intrinsic resistance of triple negative breast cancer (TNBC) to CDK4/6 inhibitors and that inhibition of the RANK pathway through knockdown expression of RANK restores sensitivity to CDK4/6 inhibitor therapy.

In the studies described in Examples 2-4, luminal breast cancer cells engineered to stably overexpress RANK were used to demonstrate a relationship between RANK expression levels and sensitivity to CDK4/6 inhibitors. Triple negative breast cancer (TNBC) has previously shown resistance to CDK4/6 inhibitor therapy (Richard Finn et al. Breast Cancer Res 2009. Finn R et al SABCS 2008, Finn R S, et al. Breast Cancer Res. 2009; 11(5):R77. https://doi.org/10.2147/BCTT.S46725 a review). Thus, the sensitivity to CDK4/6 inhibitors exhibited by MDA-MB-231 TNBC cells was compared to that of RANK knock-down (KD) clones.

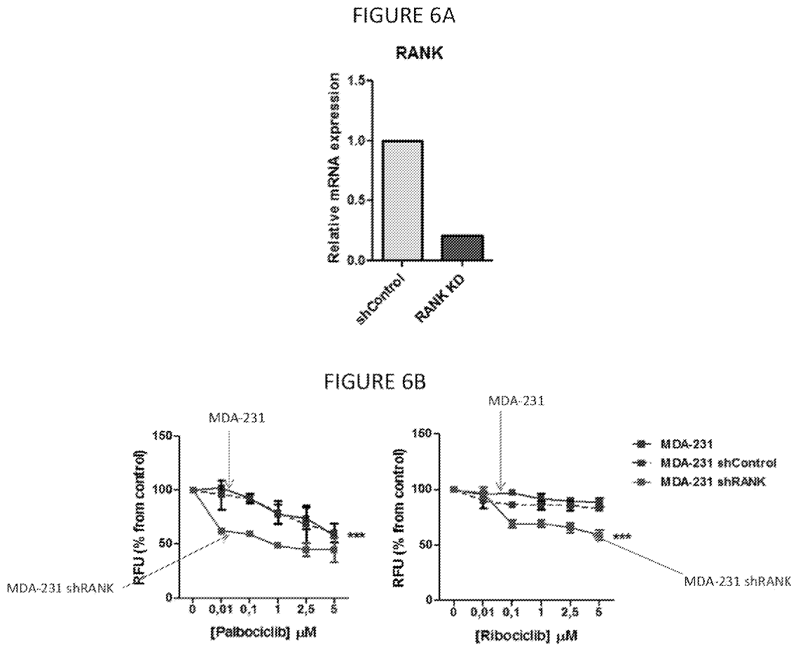

RANK KD clones were first generated for use in sensitivity assays. Briefly, MCF-7GFP+Luc+ cells were seeded in multi-well plates and then exposed to fresh medium comprising RANK shRNA (h) lentiviral particles or control shRNA lentiviral particles. The transduced cells were selected using Puromycin dihydrochloride starting three days post-transduction. Knockdown of RANK expression by cells exposed to RANK shRNA lentiviral particles was confirmed by RT-qPCR and the level of RANK expression of these cells were compared to RANK expression by cells transduced with control shRNA lentiviral particles (FIG. 6A). Sensitivity to CDK4/6 inhibitors was tested as essentially described in previous examples but TNBC RANK KD cells (MDA-231 shRANK), non-transduced TNBC cells (MDA-231) or control lentiviral particle-transduced (MDA-231 shControl) cells were used and the results of the assay are shown in FIG. 6B. As shown in FIG. 6B, both MDA-231 cells and MDA-231 shControl cells demonstrated a resistance to CDK4/6 inhibitors palbociclib and ribociclib. Interestingly, TNBC RANK KD cells demonstrated restored sensitivity to both of these CDK4/6 inhibitors, in comparison to the RANK expressing cells (MDA-231 cells and MDA-231 shControl cells).

### Example 6

This example demonstrates that the RANK pathway contributes to the intrinsic resistance of triple negative breast cancer (TNBC) to CDK4/6 inhibitors and that inhibition of the RANK pathway restores sensitivity to CDK4/6 inhibitor therapy.

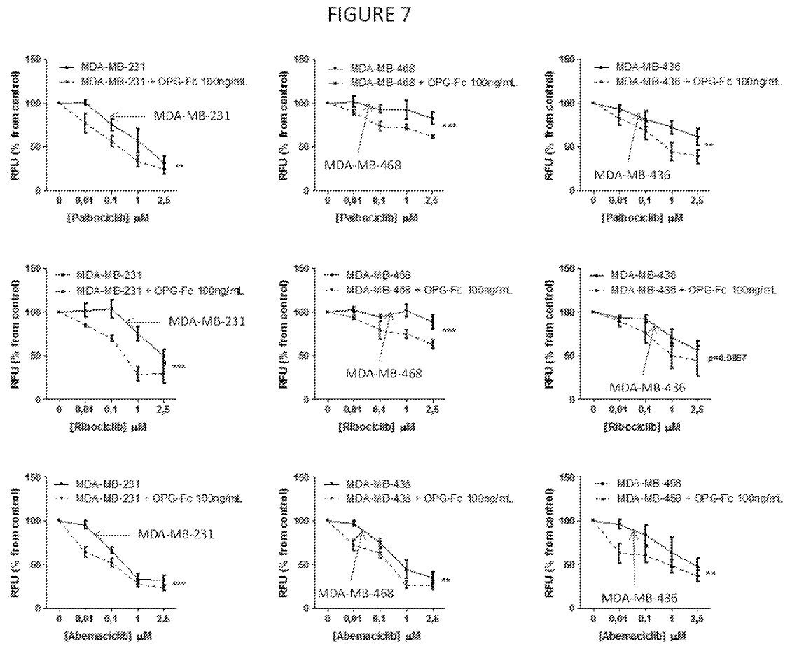

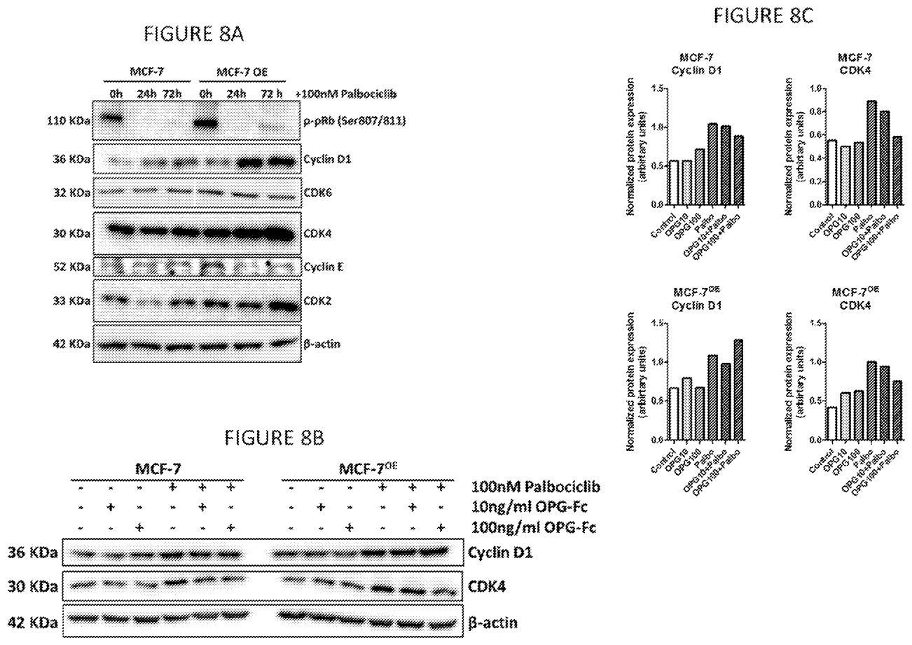

Sensitivity to CDK4/6 inhibitors palbociclib, ribociclib and abemaciclib was tested as essentially described in previous examples except that TNBC cells (MDA-MB-231, MDA-MB-436, MDA-MB-468) were used, instead of LBC cells. TNBC cells were exposed to CDK4/6 inhibitors alone or in combination with the RANK pathway inhibitor, OPG-Fc. The results are shown in FIG. 7.

As shown in FIG. 7, TNBC cells demonstrated a resistance to each of the CDK4/6 inhibitors tested. Interestingly, when the cells were exposed to the CDK4/6 inhibitor in combination with the RANK pathway inhibitor (OPG-Fc), the sensitivity to the CDK4/6 inhibitor was restored, as shown by reduced cell viability relative to the cells treated with the CDK4/6 inhibitor without OPG-Fc.

RANK-pathway blockage could also sensitize TNBC cells to CDK4/6 inhibitors. Using a panel of three TNBC cell lines and the three available CDK4/6 inhibitors, we demonstrate that RANK pathway mediates resistance to palbociclib, ribociclib and abemaciclib, since osteoprotegerin (OPG-Fc) restored sensitivity to therapy (FIG. 7).

### Example 7

This example demonstrates that blockade of the RANK pathway abrogates up-regulated expression of proteins involved in cyclin/CDKs complexes.

To understand the reason why RANK OE cells are less sensitive to CDK4/6 inhibitors, the expression of proteins involved in cyclin/CDKs complexes were analyzed. Briefly, expression of cyclin D1, CDK4, CDK6, Cyclin E and CDK2 were analyzed by Western blotting cell lysates of RANK OE (MDF-7 OE) cells exposed to palbociclib for 0 hours, 24 hours or 72 hours, and compared to the corresponding expression levels in the parental counterpart cells (expressing low levels of RANK; MCF-7 cells). RANK OE cells demonstrate increased expression of cyclin D1, CDK4, Cyclin E and CDK2, compared to the parental counterpart cells, and, CyclinD1, CDK4 and CDK2 were increased upon exposure to a CDK4/6 inhibitor.

The same assay was carried out with RANK OE cells and the corresponding parental cells expressing low levels of RANK except this time the cells were exposed to palbociclib for seven days alone or in combination with OPG-Fc at either 10 ng/mL or 100 ng/mL. When the RANK pathway was inhibited with OPG-Fc, RANK OE cells no longer exhibited the up-regulation of CDK4. These data suggest that the up-regulation of CDK4 involved in cyclin/CDKs complexes is abrogated when the RANK pathway is inhibited.

### Example 8

This example demonstrates the characterization of in vivo growth of luminal BC cells engineered to overexpress RANK.

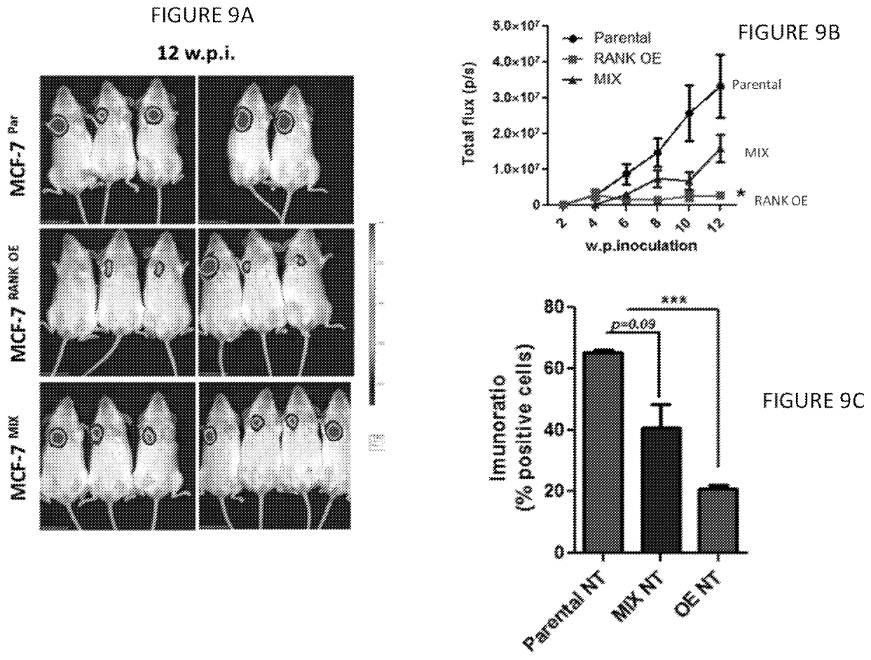

To determine the effects of RANK expression on the in vivo growth of cancer cells, Nod Scid Gamma (NSG) mice were inoculated in the 2^{nd} thoracic mammary fat pad with (1) MCF-7GFP+Luc+ (Parental) cells, (2) MCF-7 RANK OE GFP+Luc+ (RANK OE) cells or (3) a combination thereof (MCF-7 GFP+Luc+ and MCF-7 RANK OE at a 1:1 ratio) (Mix). In this study, each group contained 5 mice. The resulting tumors formed from the inoculated cancer cells were imaged by bioluminescence every week post-tumor inoculation tills the end of the study (twelve weeks). A series of representative images of tumors at 12 weeks post-inoculation is shown in FIG. 9A, the total flux, which is representative of emitted photons/s and allows the quantification of tumor burden, is shown in FIG. 9B, and the percentage of Ki67-positive cells, which represents an index of proliferation, is shown in FIG. 9C. As shown in these figures, tumors formed from cells overexpressing RANK exhibited a decreased growth and proliferation rate.

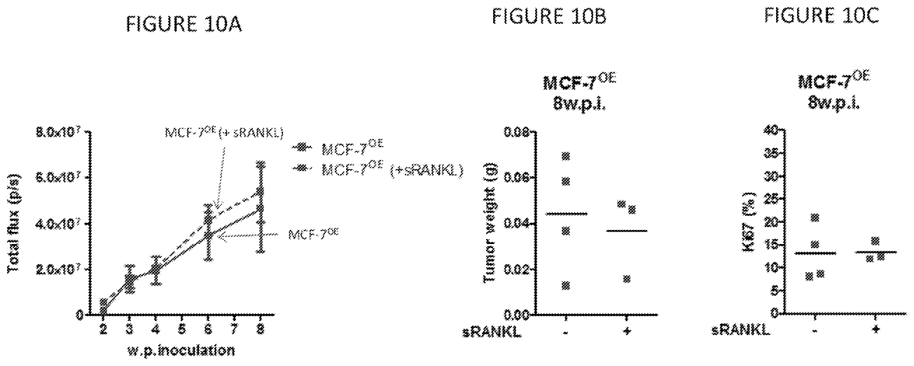

To determine whether the decreased proliferation rate was due to RANKL availability, NSG mice were inoculated with one of Parental, RANK OE, or Mix cells, as described above, and treated with human soluble RANKL or vehicle control subcutaneously. As shown in FIGS. 10A-10C, the administration of sRANKL did not affect the tumor growth.

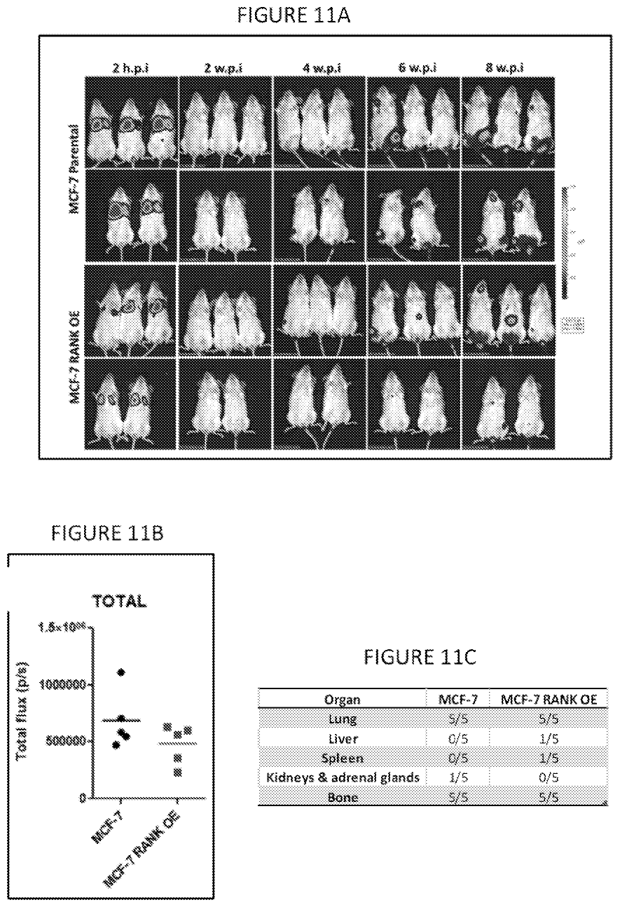

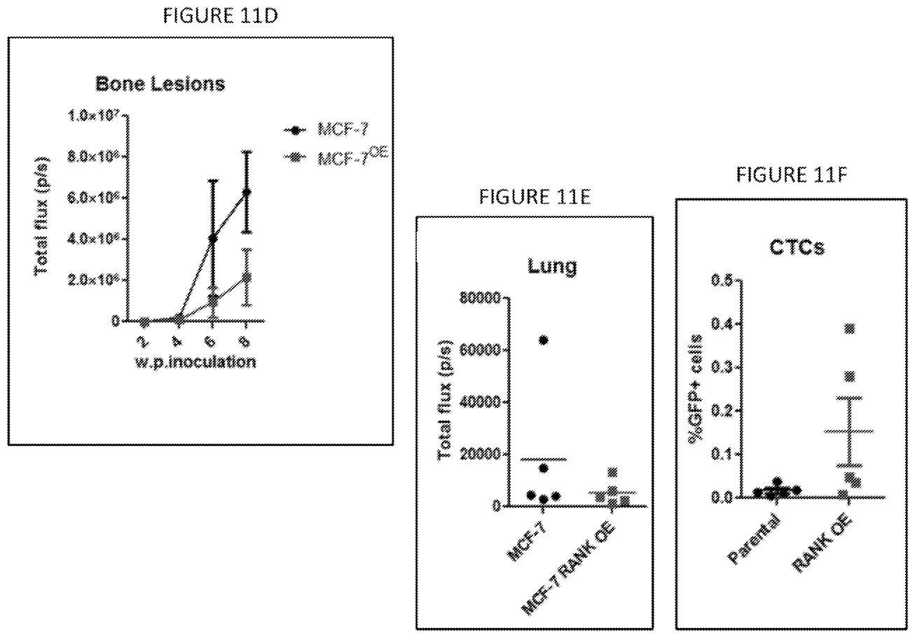

To determine if cells overexpressing RANK were more invasive in vivo, an experimental metastases model was performed. Briefly, NSG mice were inoculated in the tail vein with Parental or RANK OE cells. Each group contained 3 mice. Lung seeding was confirmed by bioluminescence 2 hours post-inoculation and the resulting tumors formed from the inoculated cancer cells were imaged every week post-inoculation until the end of the experiment. A series of representative images of tumors at the indicated timepoint relative to time post-inoculation (p.i.) is shown in FIG. 11A, the total flux is shown in FIG. 11B, the macrometastases (as assessed by ex vivo bioluminescence at necropsy) is shown in FIG. 11C, and the total flux on bone lesions, total flux on lung, and the number of circulating tumor cells (CTC) are shown in FIGS. 11D, 11E, and 11F, respectively. As shown in these figures, RANK OE cells were equally metastatic in lungs and bones, relative to the parental cell line expressing low levels of RANK, but mice inoculated with RANK OE cells exhibited an increased number of CTCs. The latter observation is consistent with a more mesenchymal phenotype, and reinforces an aggressive potential.

### Example 9

This example demonstrates the in vivo sensitivity to CDK4/6 inhibitors in combination with hormone therapy of LBC cells engineered to overexpress RANK.

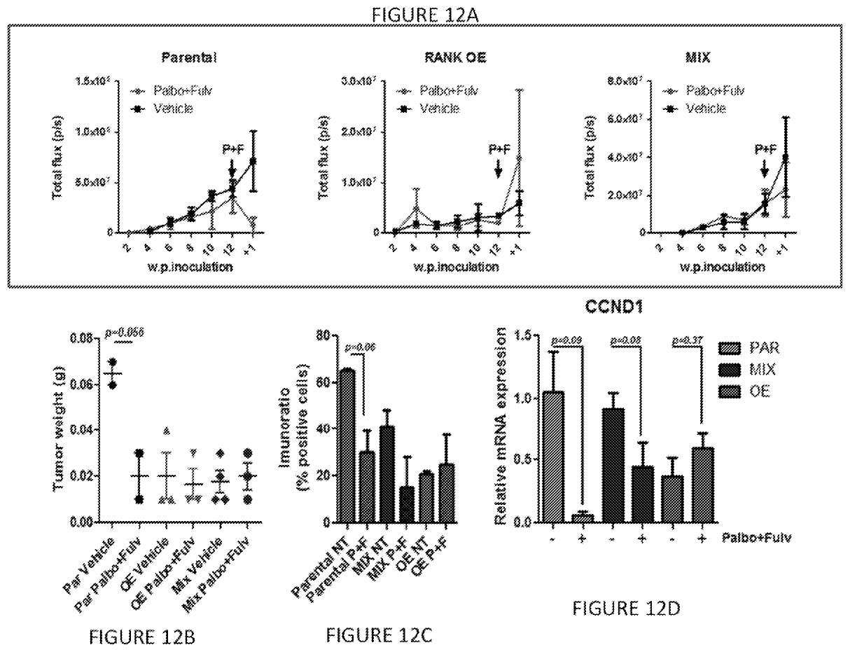

NSG mice were inoculated in the 2^{nd} thoracic mammary fat pad with (1) MCF-7GFP+Luc+ (Parental) cells, (2) MCF-7 RANK OE GFP+Luc+ (RANK OE) cells or (3) a combination thereof (MCF-7 GFP+Luc+ and MCF-7 RANK OE at a 1:1 ratio) (Mix). In this study, each group contained 3-4 mice. The resulting tumors formed from the inoculated cancer cells were imaged by bioluminescence every week post-tumor inoculation tills the end of the study. At 12-weeks post-inoculation, mice were randomized into groups based on tumor size and treated with (A) a tumor regressing dose of palbociclib (based on Vijayaraghavan et al., Nature Communications 8, article number 15916 (2017); doi:10.1038/ncomms15916) for 7 days in combination with Fulvestrant or (B) vehicle control. The total flux is shown in FIG. 12A, the tumor weight at necropsy is graphed in FIG. 12B, the percentage of Ki67-positive cells (which represents an index of proliferation) is shown in FIG. 12C, and the expression of CCND1 (Cyclin D1) is shown in FIG. 12D. As shown in FIG. 12A, combination treatment (palbociclib and fulvestrant) was able to induce a regression in MCF-7 xenografts but not on MCF-7^{OE} or mixed xenografts (FIG. 12A). The treatment on MCF-7 xenografts was confirmed by decreased tumor weight at sacrifice (as shown in FIG. 12B), a decreased proliferation index (as shown in FIG. 12C), and a decreased CCND1 expression (as shown in FIG. 12D), in comparison with tumors not treated with palbociclib and fulvestrant. These data demonstrate that tumors formed from cells overexpressing RANK exhibited resistance to combination therapy with palbociclib and fulvestrant in vivo.

### Example 10

This example demonstrates proof-of-concept that in vivo RANK pathway inhibition sensitizes breast cancer cells to CDK4/6 inhibitors.

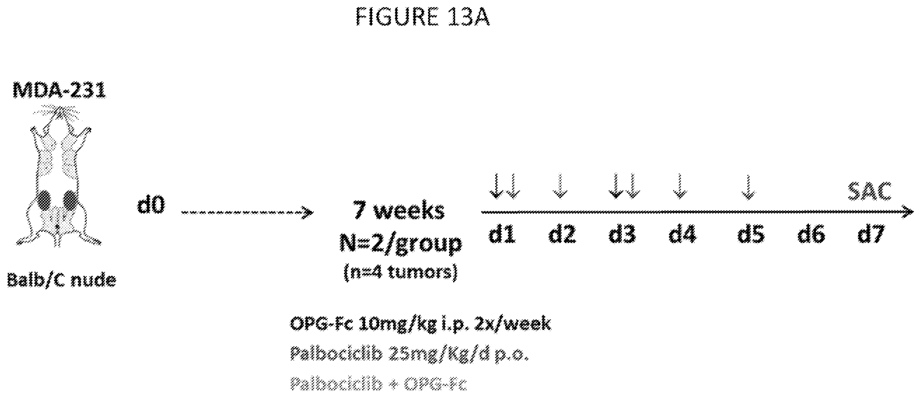

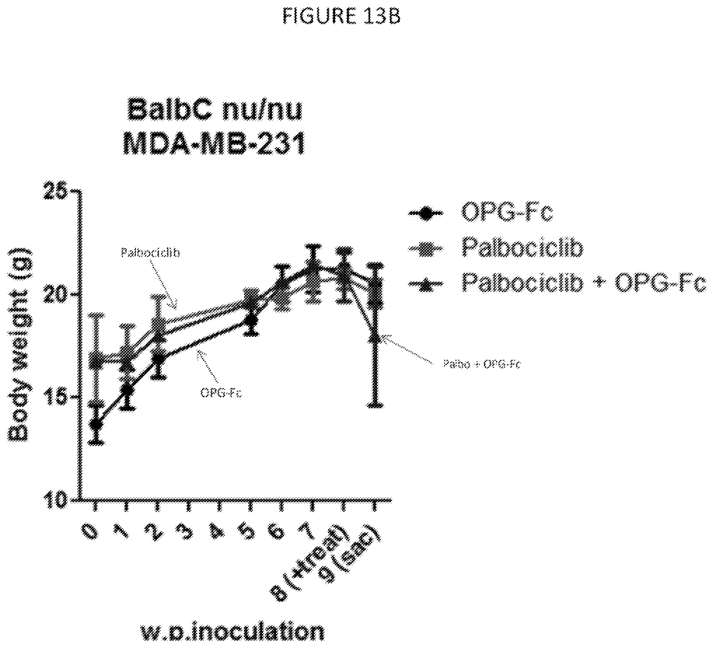

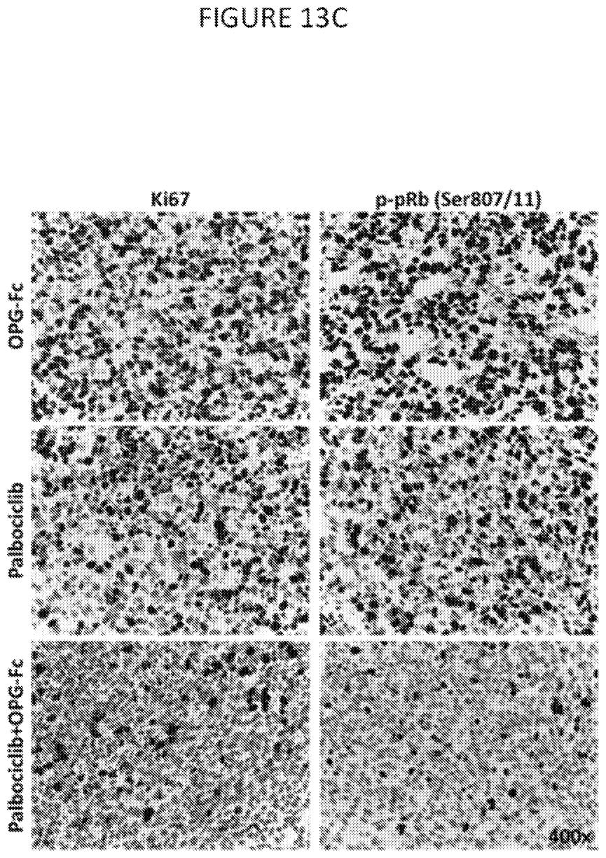

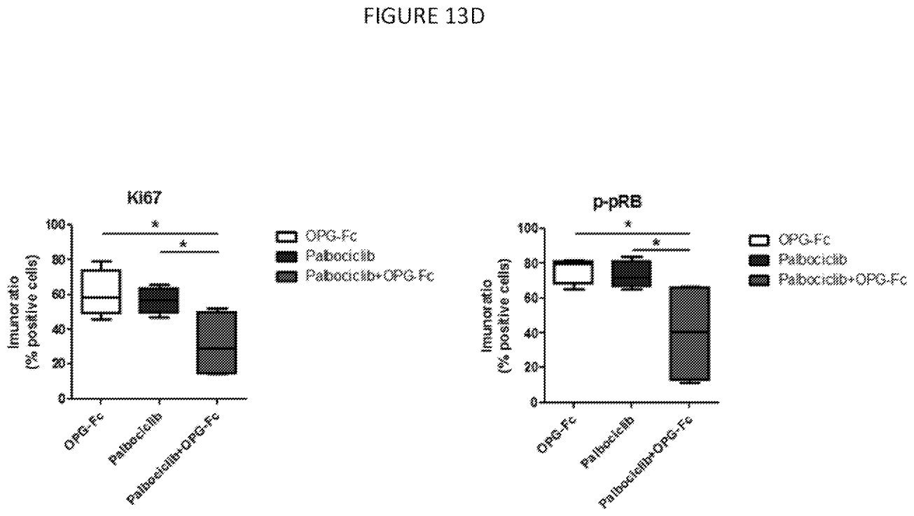

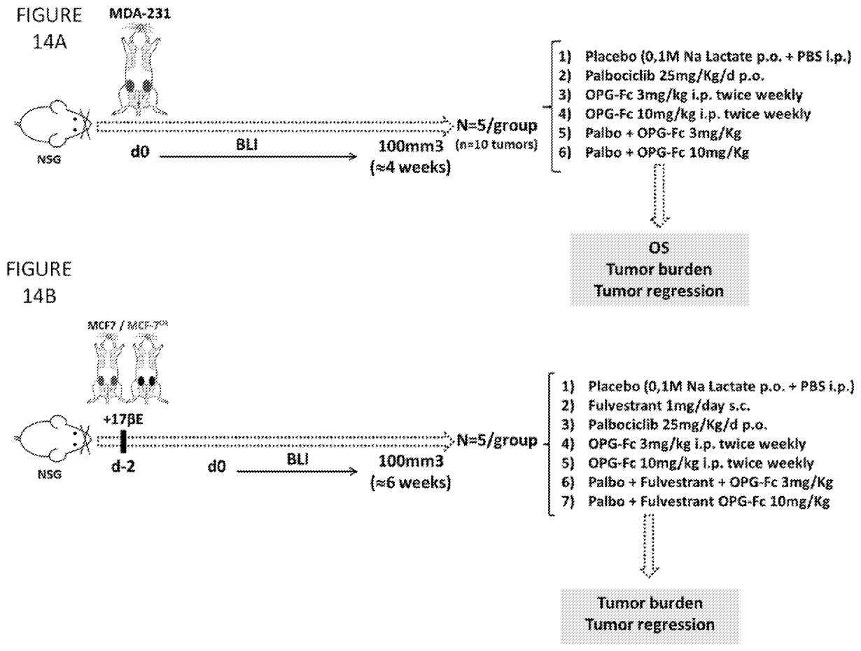

A pilot study to test the sensitivity of TNBC xenografts to CDK4/6 inhibitors (palbociclib) in combination with RANK pathway blockage (OPG-Fc) was performed. Briefly, NSG mice were inoculated bilaterally in the 4^{th} abdominal mammary fat pad with MDA-MB-231 GFP+Luc+ cells (n=2/group) Seven weeks post inoculation, mice were randomized into groups based on tumor size and then treated with (A) OPG-Fc (10 mg/Kg i.p. twice per week); (B) Palbociclib (25 mg/Kg/day p.o.) or (C) a combination thereof. A schematic of the experimental design is shown in FIG. 13A. Body weight was measured throughout the experiment and the results are shown in FIG. 13B. As shown in this figure, body weight was not different between groups. The proliferation index of tumors as measured by the quantification of Ki67-positive cells and p-pRB positive cells is shown in FIG. 13D. FIG. 13C is a series of images of Ki67-stained or p-pRb stained cells. As shown in these figures, tumors treated with palbociclib plus OPG-Fc demonstrated a significant decrease in Ki67 and p-pRB expression. These data support that RANK pathway blockade sensitizes triple negative tumors to treatment with CDK 4/6 inhibitors, such as palbociclib.

### Example 11

This example describes the design of two studies for testing sensitivity to CDK4/6 inhibitors in combination with RANK pathway blockade in both luminal breast cancer and triple negative breast cancer.

In the first study, for TNBC, NSG mice are inoculated bilaterally in the 4^{th} abdominal mammary fat pad with MDA-MB-231 GFP+Luc+ cells (n=5/group). After tumor inoculation, tumors are imaged by bioluminescence every week till tumors reach approximately 100 mm³ (e.g., ˜4 weeks). When tumors reach approximately 100 mm³, mice are randomized into groups based on tumor size and treated with (1) placebo (0.1 M Sodium lactate p.o.+PBS i.p.), (2) palbociclib (25 mg/kg/d p.o.), (3) OPG-Fc (3 mg/kg i.p. 2× weekly); (4) OPG-Fc (10 mg/kg i.p. 2× weekly); (5) a combination of (2) and (3); or (6) a combination of (2) and (4). A schematic of the experimental design is shown in FIG. 14A.

A second study, for LBC, NSG mice are supplemented with 17β-estradiol pellets two days before inoculation with (1) MCF-7GFP+Luc+ (Parental) or (2) MCF-7 RANK OE GFP+Luc+(RANK OE). After tumor inoculation, tumors are imaged by bioluminescence every week till tumors reach approximately 100 mm³ (e.g., ˜4 weeks). When tumors reach approximately 100 mm³, mice are randomized into groups based on tumor size and treated with (1) placebo (0.1 M Sodium lactate p.o.+PBS i.p.), (2) palbociclib (25 mg/kg/d p.o.), (3) OPG-Fc (3 mg/kg i.p. 2× weekly); (4) OPG-Fc (10 mg/kg i.p. 2× weekly); (5) a combination of (2) and (3); or (6) a combination of (2) and (4). A schematic of the experimental design is shown in FIG. 14B.

Overall survival (OS), tumor burden and tumor regression is measured at the end of the study.

In these studies, TNBC (FIG. 14A) and estrogen receptor positive (ER+) breast cancer (Luminal BC) (FIG. 14B) sensitivity to CDK4/6 inhibitors (palbociclib) in combination with RANK pathway blockade are tested. It is anticipated that combination of CDK4/6 inhibitors with RANK pathway blockage will increase tumor regression, and increase overall survival.

### Example 12

This example describes the design of a clinical trial to determine the safety and efficacy of the combination of denosumab and CDK 4/6 inhibitors for the treatment of human subjects with triple negative breast cancer or luminal breast cancer.

1. Triple Negative Breast Cancer (TNBC)

Characteristics of eligible patients for the TNBC study include age 18 or older, with triple negative breast cancer, unresectable or metastatic, with measurable disease at the extraskeletal site as per RECIST 1.1, who have received at least two chemotherapy regimens for advanced disease and not more than five, including an anthracycline and a taxane. Patients are to have adequate bone marrow, liver and renal function and an Eastern Cooperative Oncology Group (ECOG) performance status of 0-2, as well as a life expectancy of three months or more.

Characteristics of ineligible patients (e.g., key exclusion criteria) include the use of any investigational drug or treatment within four weeks of the study; the use of chemotherapy or radiation within three weeks of the study; known brain metastases unless treated and stable, and previous treatment with denosumab

All patients are to provide written informed consent and study approval is obtained from the central ethics committee. All procedures accord with the World Medical Association (WMA) Declaration of Helsinki revised edition (WMA General Assembly, Tokyo, 2004), the International Conference for Harmonisation (ICH) of technical requirements for registration of pharmaceuticals for human use, good clinical practice and local ethical and legal requirements.

The purpose of this trial protocol is to assess the activity of denosumab in the standard dose of 120 mg q4w in combination with a CDK4/6 inhibitor in a phase 2 clinical trial. Additional regimens to be tested include 120 mg every 2 weeks for the first two months followed by the regimen of 120 mg q4w. Treatment is carried out until disease progression, death, intolerable toxicity, patient's withdrawal of consent or a clinician's decision to withdraw the patient.

The primary endpoint of this study is Disease Control Rate (DCR) which is defined by the total of patients with stable disease (SD), partial response (PR) or complete response (CR) as per RECIST 1.1. Secondary endpoints include the following:

| | |
|---|---|
| Characteristic | Defined by or as demonstrated by |
| Objective | the total of patients with CR or PR as per RECIST 1.1; |
| Response | |
| Rate (ORR) | |
| Progression- | the time between the start of therapy and progression or |
| Free Survival | death by any cause |
| (PFS) | |
| Overall | the time between the start of therapy and death by any |
| Survival | cause |
| (OS) | |
| Time to | the time between the start of therapy and first decrease |
| Deterioration | in ECOG score; |
| of ECOG | |
| Safety | Adverse effects (according to Common Terminology |
| | Criteria for Adverse Events (CTCAE v5.0; U.S. Dept. of |
| | Health and Human Services (published Nov. 27, 2017, |
| | https://ctep.cancer.gov/protocoldevelopment/electronic_ |
| | applications/docs/ctcae_v5_quick_reference_5x7.pdf.) |

The study is carried out according to one of the following trial designs:
Single arm, Simon's Two Stage Design in which all patients are treated with a CDK4/6 inhibitor and denosumab at 120 mg q4w. Historical comparator of the Treatment Physicians of Choice (TPC) arm of the EMBRACE trial (Eribulin; The Lancet, Volume 377, ISSUE 9769, P914-923, Mar. 12, 2011
DOI:https://doi.org/10.1016/SO140-6736(11)60070-6); or
Randomized 2:1 between CDK inhibitor+Denosumab 120 mg q4w and Treatment of Physicians' choice (TPC) to allow for removal of selection bias and confounding (data from the EMBRACE trial is from the first decade of 2000);
   Patients will be stratified for the number of previous chemotherapy regimens (2 or >2) and metastasis to the liver/lungs (yes/no).

2. Luminal Breast Cancer (LBC)

Characteristics of eligible patients for the LBC study include age 18 or older, with either Estrogen Receptor(ER)/Progesterone Receptor(PR) positive, c-erbB2-negative breast cancer, unresectable or metastatic with measurable disease (at extraskeletal site) as per RECIST 1.1, who have not previously received CDK4/6 inhibitors or endocrine therapy (ET). One chemotherapy regimen for advanced disease is allowed. Patients who are pre-menopausal have to be submitted to surgical or chemical castration.

Patients are to have adequate bone marrow, liver and renal function and an Eastern Cooperative Oncology Group (ECOG) performance status of 0-2, as well as life expectancy of three months or more.

Characteristics of ineligible patients (e.g., key exclusion criteria) include the use of any investigational drug or treatment within four weeks of the study; the use of chemotherapy or radiation within three weeks of the study; known brain metastases unless treated and stable, or previous treatment with denosumab.

All patients are to provide written informed consent and study approval is obtained from the central ethics committee. All procedures accord with the World Medical Association (WMA) Declaration of Helsinki revised edition (WMA General Assembly, Tokyo, 2004), the International Conference for Harmonisation (ICH) of technical requirements for registration of pharmaceuticals for human use, good clinical practice and local ethical and legal requirements.

The purpose of this trial protocol is to assess the activity of denosumab in the standard dose of 120 mg q4w in combination with a CDK4/6 inhibitor in a phase 2 clinical trial. Additional regimens to be tested include 120 mg every 2 weeks for the first two months followed by the regimen of 120 mg q4w. Treatment will be done until progression, death, intolerable toxicity, clinician's decision or patient withdrawal of consent.

The primary endpoint of this study is Objective Response Rate (ORR) in Extraskeletal Disease (ESD) defined by the total of patients with stable disease (SD) or partial response (PR) as per RECIST 1.1. Secondary endpoints include the following:

| | |
|---|---|
| Characteristic | Defined by or as demonstrated by |
| Disease | the total of patients with SD, CR or PR as per |
| Control | RECIST 1.1; |
| Rate (DCR) | |
| Progression- | the time between the start of therapy and progression or |
| Free Survival | death by any cause |
| (PFS) | |
| Overall | the time between the start of therapy and death by any |
| Survival (OS) | cause |
| Time to | the time between the start of therapy and first decrease |
| Deterioration | in ECOG score; |
| of ECOG | |
| Safety | Adverse effects (according to Common Terminology |
| | Criteria for Adverse Events (CTCAE v5.0; U.S. Dept. of |
| | Health and Human Services (published Nov. 27, 2017, |
| | https://ctep.cancer.gov/protocoldevelopment/electronic_ |
| | applications/docs/ctcae_v5_quick_reference_5x7.pdf.) |

The trial design includes the following features: (1) Patients are stratified for the presence of lung/liver metastasis (yes/no), previous chemotherapy and endocrine treatment choice (NSAI/SERM); (2) Patients without bone metastasis are randomised 2:1 between CDK4/6 inhibitor+ET and CDK4/6 inhibitor+ET+Denosumab 120 mg q4w; (3) Patients with bone metastasis are randomised 2:1 between CDK4/6 inhibitor+ET+Zoledronic Acid and CDK4/6 inhibitor+ET+Denosumab 120 mg q4w.

### Example 13

This example describes experiments designed to test sensitivity to an inhibitor which blocks CDK4 or CDK6 in combination with RANK pathway blockade in both luminal breast cancer and triple negative breast cancer.

This example describes experiments designed to test sensitivity to an inhibitor which blocks CDK4 in combination with RANK pathway blockade in both luminal breast cancer and triple negative breast cancer.

The sensitivity to CDK4 inhibitors is tested as essentially described in previous examples (Examples 2-6) using LBC cells (e.g., MCF-7 and T47D) and TNBC cells (MDA-MB-231, MDA-MB-436, MDA-MB-468). LBC and TNBC cells are seeded in multi-well plates and then exposed to a CDK4 inhibitor, alone or in combination with the RANK pathway inhibitor, OPG-Fc.

The CDK4 inhibitors that are used in this assay include 3-ATA (3-Amino-9-thio(10H)-acridone) and Cdk4 Inhibitor III (5-(N-(4-Methylphenyl)amino)-2-methyl-4,7-dioxobenzothiazole, Ryuvidine). Viability of cells is assessed by Alamar blue assay, as described herein.

To test the sensitivity to CDK4 inhibitors of TNBC or LBC cells in vivo, NSG mice are inoculated bilaterally in the 4th abdominal mammary fat pad with MCF-7GFP+Luc+ (Parental), MCF-7 RANK OE GFP+Luc+ (RANK OE), or MDA-MB-231 as essentially described herein. Twelve weeks after inoculation, mice are randomized into groups based on tumor size and treated with (A) CDK4 inhibitor, (B) OPG, (C) a combination of (A) and (B), or (D) a vehicle control. Tumors are imaged by bioluminescence every week post inoculation until the end of the study. Total flux and quantification of Ki67-positive and phospho-Rb-positive cells are measured as described herein.

### Example 14

This example describes experiments designed to test loss or decrease of CDK4 or CDK6 expression in combination with RANK pathway blockade in growth of both luminal breast cancer and triple negative breast cancer.

In one set of experiments, cells are transduced with lentiviral vectors comprising a shRNA that targets CDK4. In another set of experiments, cells are transduced with lentiviral vectors comprising a shRNA that targets CDK6. In both cases control shRNA are used.

LBC and TNBC cells with CDK4 or CDK6 knock-down (decreased expression in comparison with control) or knock-out (total loss of expression in comparison with control), or control shRNA, are seeded in multi-well plates and then exposed to RANK pathway inhibitor, OPG-Fc. Cell viability is assessed by Alamar blue assay, as described herein.

To test loss or decrease of CDK4 or CDK6 expression in combination with RANK pathway blockade in growth of both luminal breast cancer and triple negative breast cancer in vivo, NSG mice are inoculated bilaterally in the 4th abdominal mammary fat pad with MCF-7GFP+Luc+ (Parental), MCF-7 RANK OE GFP+Luc+ (RANK OE), MDA-MB-231, or their CDK4, CDK6, control shRNA knock-down counterparts as essentially described herein. Twelve weeks after inoculation, mice are randomized into groups based on tumor size and treated with (A) OPG or (B) a vehicle control. Tumors are imaged by bioluminescence every week post inoculation until the end of the study. Total flux and quantification of Ki67-positive and phospho-Rb-positive cells are measured as described herein.

### Example 15

This example describes elevated RANK expression in estrogen receptor-positive breast cancer is a driver of stemness.

Background: The receptor activator of NFkB (RANK)-RANK ligand (RANKL) pathway, pivotal regulator of bone remodeling, has emerged as a major mediator of progesterone-driven breast cancer (BC) carcinogenesis. RANK expression is particularly elevated in triple negative BC and has been associated with aggressiveness and poor prognosis. This study addressed the impact of elevated RANK expression in estrogen receptor (ER)-positive BC.

Methods: In silico analysis of RANK (TNFRSF11A) expression was performed in the TCGA BC cohort. ER+HER2− cell lines overexpressing RANK were obtained by lentiviral transduction and standard assays were used for gene expression and phenotype evaluation. In vivo tumor growth was measured in orthotopic xenografts or after tail vein inoculation. Comparisons between two groups were performed using unpaired t-test. Survival analyzes used the Kaplan-Meier method and log-rank test.

Results: RANK expression was higher in ER− breast tumors (p<0.001); and associated with decreased 5-year overall survival (HR=2.12 (1.16-3.87), p=0.012). However, 5% of ER+ tumors were found to express RANK within the ER− 75% Q range. ER+ cell lines overexpressing RANK (MCF-70E and T47DOE) were characterized by hyper activation of downstream pathways; upregulation of mesenchymal and stem cell markers; increased sphere forming capacity. In silico, elevated RANK expression in ER+ tumors was significantly correlated with mesenchymal, stemness. In vivo, MCF-70E xenografts were consistently smaller in comparison with parental ones. However, tail vein inoculation showed that MCF-7 and MCF-70E cells where identically metastatic in the lungs and skeleton, and animals harboring MCF-70E cells had more circulating tumor cells.

Conclusions: Our data shows for the first time that RANK expression in ER+BC is not unneglectable, and may impact on the tumor progression, particularly in the metastatic setting.

### Example 16

This example demonstrates the biological features of estrogen receptor-positive breast cancer with elevated RANK expression.

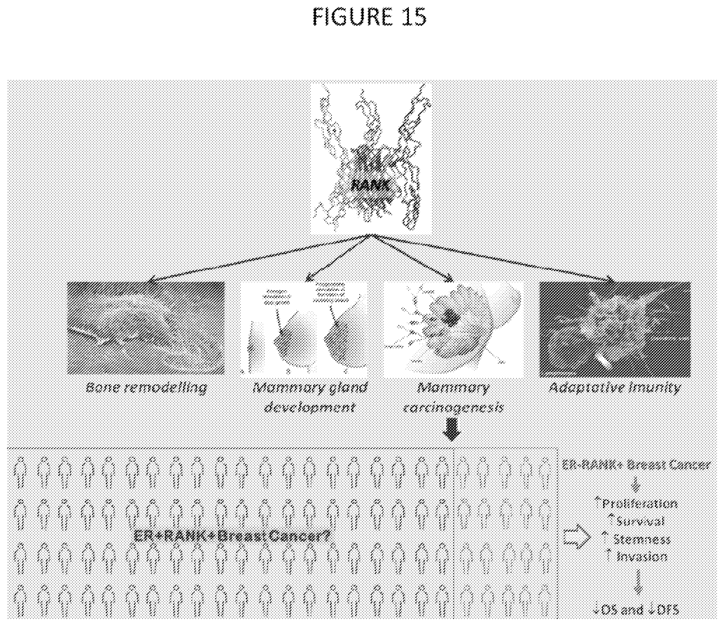

Background: The RANK-RANKL pathway is the pivotal regulator of bone remodeling. In the past decade RANK-RANKL axis has emerged as a major mediator of progesterone-driven breast cancer (BC) carcinogenesis. RANK expression is particularly elevated in triple negative breast cancer (TNBC) and has been associated with aggressiveness and poor prognosis. RANK expression in Luminal BC was not previously assessed. See FIG. 15. The aim of this study was to determine what is the biological impact of elevated RANK expression in ER-positive (ER+) breast cancer.

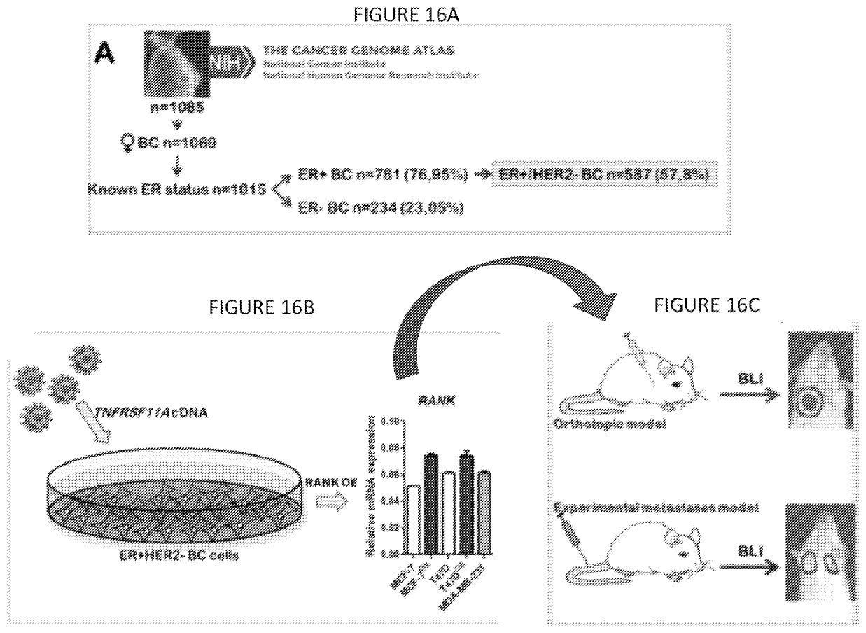

The methodology of this study included an in silico analysis of RANK expression performed on The Cancer Genome Atlas (TCGA) breast cancer (BC) cohort. The TCGA BC cohort was used to quantify RANK and further interest genes' expression. FIG. 16A. ER+HER2-negative (HER2-) cell lines overexpressing RANK were obtained by lentiviral transduction and standard assays were used for gene expression and phenotype evaluation. Human breast carcinoma cell lines MCF-7GFP+Luc+ and T47D cells were transduced with RANK lentiviral overexpression particles (EX-00007-Lv121, GeneCoepoeia) and RANK overexpression (RANK OE) was confirmed by RT-qPCR. FIG. 16B. In vivo tumor growth was measured in orthotopic xenografts or after tail vein inoculation. All animal experiments were licensed by the DGAV; and animals were handled and euthanized in accordance with approved institutional, national and international guidelines, applying the Principle of the 3Rs. 4-week old NSG mice were supplemented with sic. 1713-estradiol pellets. 2)(105 cells/ml in 50% phenol-free matrigel solution (Corning) and were injected into the second thoracic fat pad or tail vein, and tumor growth was monitored weekly by luminescence analysis. FIG. 16C.

Results

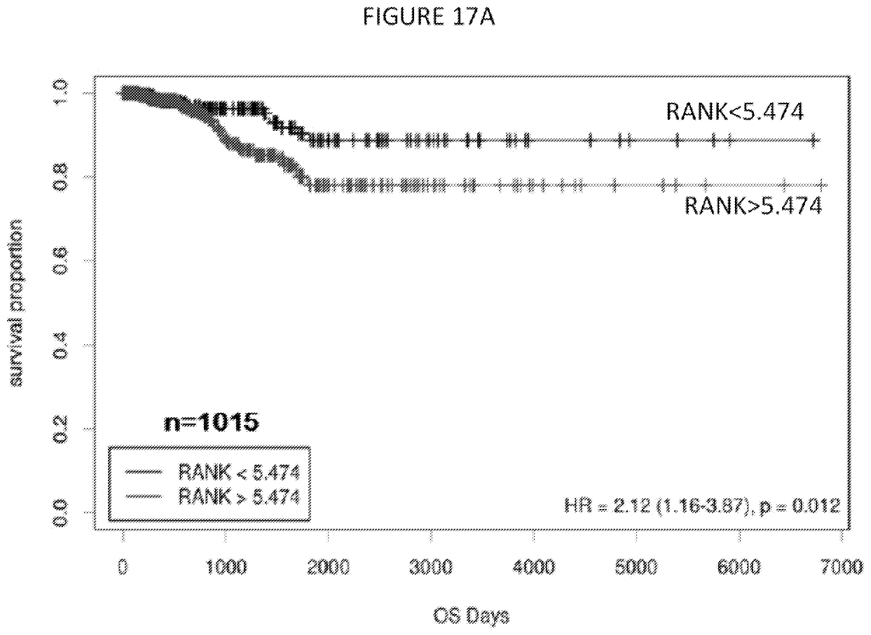

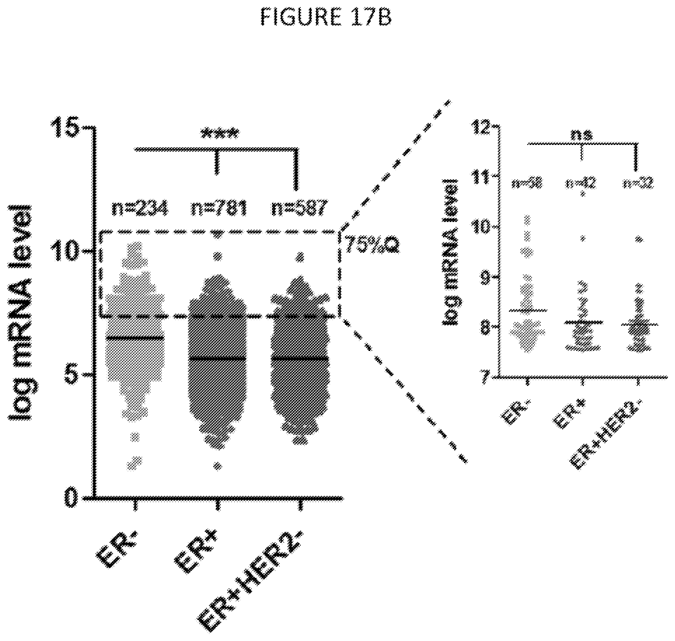

In a set of experiments, in silico analysis of RANK expression was performed in the TCGA BC cohort. As shown in FIG. 17A, female BC patients from TCGA (n=1015) were dichotomized according to RANK expression using the best cut-off (Cut-off Finder software), and survival curves plotted using the Log-rank test. FIG. 17B shows the median RANK expression was compared between ER- and ER+BC tumors; and within the 75% Q of ER-tumors. Results are presented as the mean±SEM. p-value was calculated using unpaired t-test, *p<0.05,**p<0.01,***p<0.001 These data show that RANK expression was higher in ER-breast tumors, high RANK expression (RANK-high) was associated with decreased 5-year overall survival (OS), and 5% ER+BC express RANK within the ER-75% Q range.

In a series of experiments, NSG mice were inoculated in the 2nd thoracic mammary fat pad with MCF-7, MCF-70E or a 1:1 mixture of MCF-7 and MCF-70E cells. FIG. 9A is a series of representative images from the bioluminescence analysis at the end of experiment. FIG. 9B is a graph of total flux (p/s) and FIG. 9C is a graph of the Quantification of Ki67 (Imunoratio). Data is presented as mean±SEM. p-value was calculated using 2-way ANOVA or unpaired t-test, *p<0.05, **p<0.01, ***p<0.001. These data show that MCF-70E xenografts were consistently smaller in comparison with parental ones.

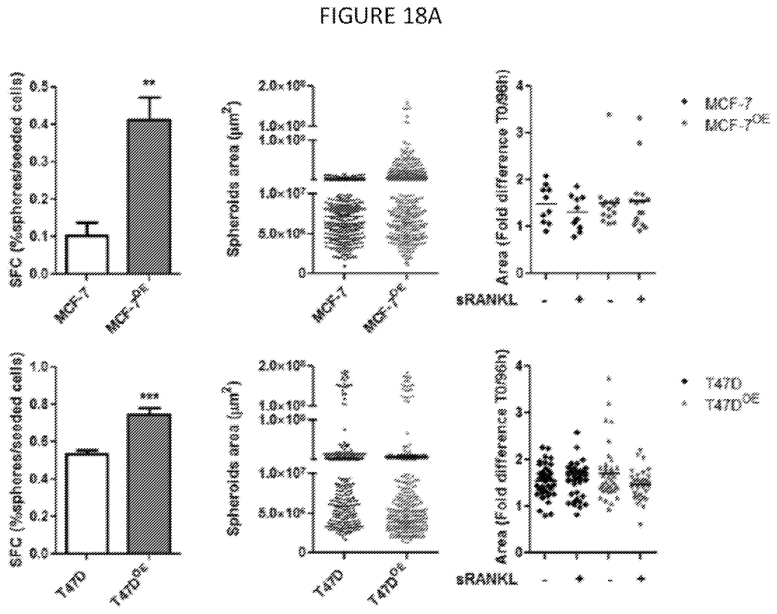

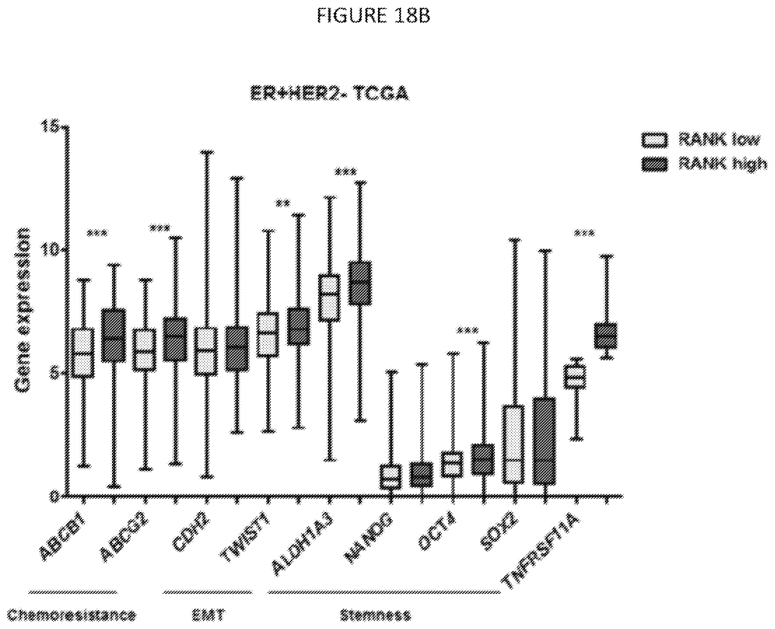

Adherent cells were cultured in non-adherent conditions and sphere forming capacity (SFC) (%) was determined as the number of mammospheres (>50 μm/number of cells seeded)×100, after 7 days spheres were measured using FiJi software and area compared in mammospheres growing with and without sRANKL (FIG. 18A). The TCGA BC cohort was used to quantify RANK and further interest genes' expression (FIG. 18B).

In a set of experiments, NSG mice were inoculated in the tail vein with MCF-7 or MCF-70E cells (n=3/group). Tumors were imaged by bioluminescence 2 h and every week post tumour inoculation till the end of the experience. FIG. 11A is a series of representative images from the bioluminescence analysis. FIG. 11B is a graph of the total flux (p/s). FIG. 11D is a graph of the total flux on bone lesion (p/s). FIG. 11E is a graph of the total flux on lung lesions (p/s). FIG. 11F is a graph of the % GFP+ cells (which represented circulating tumor cells (CTCs)) as measured by flow cytometry. In this experiment, blood was collected by cardiac puncture at sacrifice and CTCs (GFP+ cells) were quantified by flow cytometry. Data analysis was performed using FlowJo V10 software. Data is presented as mean±SEM. p-value was calculated using 2-way ANOVA or unpaired t-test, *p<0.05, **p<0.01, ***p<0.001. Here, tail vein inoculation showed that MCF-7 and MCF-70E cells were identically metastatic in the lungs and skeleton. Animals with MCF-70E xenografts had more circulating tumor cells.

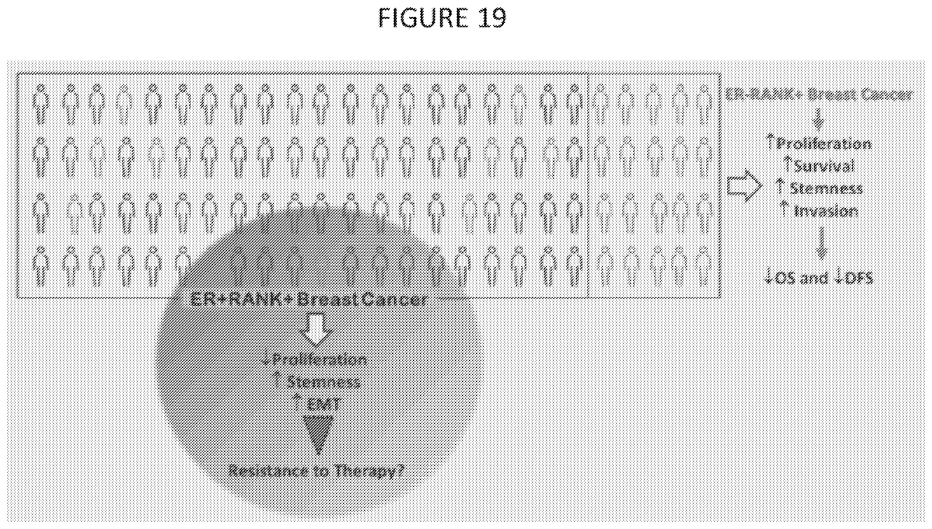

Conclusion (FIG. 19): These data show for the first time the relevance of RANK expression in ER+BC. RANK expression in ER+ tumors may impact tumor progression.

### Example 17

This example demonstrates RANK OE ER+HER2− breast cancer cells are resistant to CDK4 inhibitors.

Example 3 demonstrates RANK OE ER+HER2− cells are resistant to CDK4/6 inhibitors, namely palbociclib, ribociclib and abemaciclib; and that RANKL blockade with OPG-Fc is able to sensitize cells to these drugs. Since CDK4/6 inhibitors have different affinity profiles to CDK4 and CDK6 (Wiedemeyer V W. R. (2018) Resistance Mechanisms to Cyclin-Dependent Kinase Inhibitors. In: Yarden Y., Elkabets M. (eds) Resistance to Anti-Cancer Therapeutics Targeting Receptor Tyrosine Kinases and Downstream Pathways. Resistance to Targeted Anti-Cancer Therapeutics, vol 15. Springer, Cham), and because resistance could be related to a specific CDK, RANK OE cells were tested to see if they are equally resistant to CDK4 and/or CDK6-specific inhibitors (Example 13).

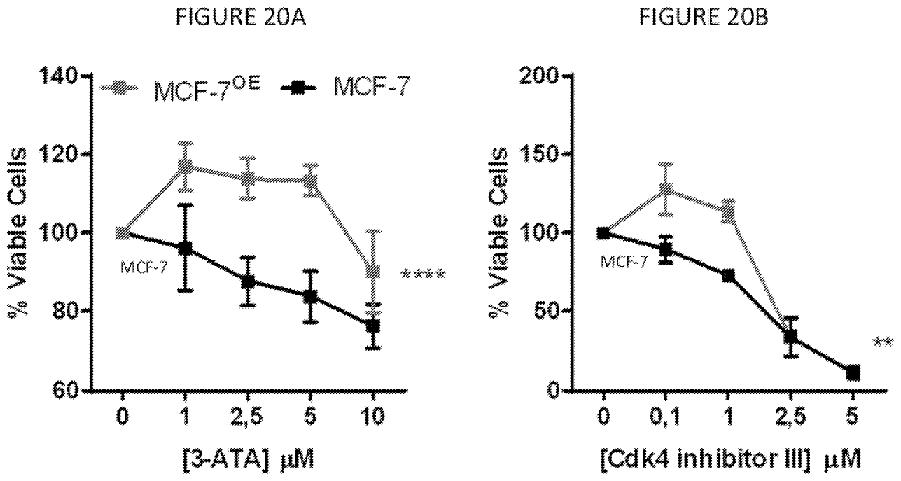

To address this question, cell growth in the presence or absence of a CDK4 inhibitor was quantified. Briefly, cells were seeded in 96-well plates and exposed to two different drugs, CDK4 inhibitors, 3-ATA (FIG. 20A) or Cdk4 inhibitor Ill (FIG. 20B) for seven days. Medium was replaced every two days, and viability was assessed by Alamar blue assay. The results are shown in FIGS. 20A and 20B. FIG. 20A provides the cell growth quantification in the presence of the CDK4 inhibitor 3-ATA and FIG. 20B provides the cell growth quantification in the presence of another CDK4 inhibitor, Cdk4 Inhibitor Ill. The results are the mean of at least three independent assays, with 4 replicates per assay, and presented as the mean±SEM. p-value was calculated using 2-way ANOVA, *p<0.05, **p<0.01, ***p<0.001.

As shown in FIGS. 20A and 208, RANK OE cells were resistant to both CDK4 inhibitors up to 2.5 μM and these cells were resistant to even higher doses of 3-ATA.

Since there are no specific CDK6 inhibitors, CDK6 knockdown or knockout RANK OE cell lines are made to address the sensitivity to specific CDK6 targeting. The cell lines are functionally down-regulated (e.g. CDK knockdown) using shRNA or knockout using gRNA/CRISPR-Cas9. Cell growth quantification of the CDK6 knockdown cell lines are compared to wild-type cell lines.

### Example 18

This example demonstrates that resistance to CDK4/6 inhibitors is not due to other CDK compensatory effects.

It was questioned whether resistance to CDK4/6 inhibitors is mediated by the compensatory activity of other CDKs, namely CDK2, which has been previously described as inducing palbociclib resistance (van der Linden M H, Willekes M, van Roon E, et al. MLL fusion-driven activation of CDK6 potentiates proliferation in MLL-rearranged infant ALL. Cell Cycle (Georgetown, Tex.). 2014; 13(5):834-844. DOI: 10.4161/cc.27757). To answer this question, cell growth in the presence of the pan-CDK inhibitor seliciclib (also known as roscovitine or CYC202) was analyzed. Briefly, cells were seeded in 96-well plates and exposed to different drugs for seven days. Medium was replaced every two days, and viability was assessed by Alamar blue assay.

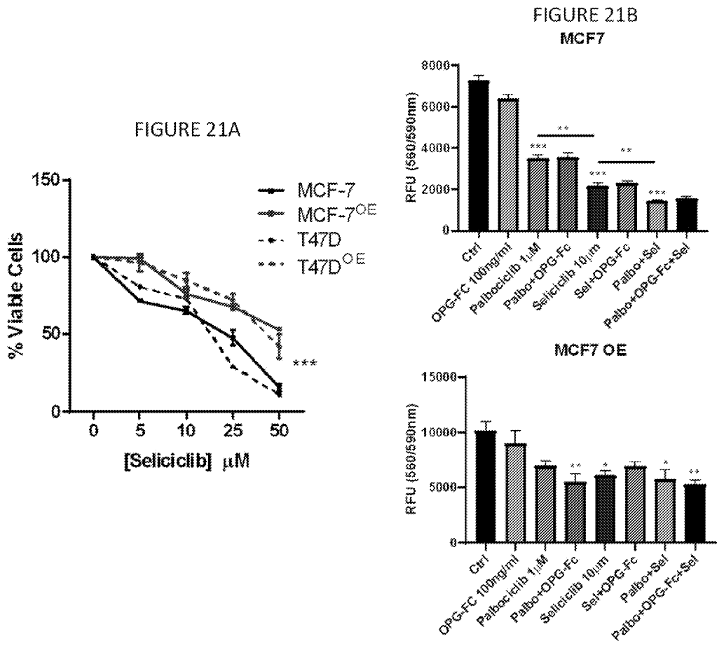

The results are shown in FIG. 21 and represent the mean of at least three independent assays, with 4 replicates per assay, and presented as the mean±SEM. p-value was calculated using 2-way ANOVA, *p<0.05, **p<0.01, ***p<0.001. Seliciclib is an inhibitor of CDK2/E, CDK2/A, CDK7 and CDK9. These data show that RANK OE cells were less sensitive to seliciclib than parental (RANK low) cells (FIG. 21A). Additionally, the combination of palbociclib plus seliciclib was more effective in parental MCF-7 cells as expected (FIG. 21B), but not in RANK OE cells, where seliciclib did not enhance the effect of palbociclib, or palbociclib plus OPF-Fc (FIG. 21B). This suggests that resistance to CDK4/6 inhibitors is not mediated by the compensatory action of other CDKs (namely, CDK2/E, CDK2/A, CDK7 and CDK9).

### Example 19

This example demonstrates OPG-Fc sensitizes TNBC cell lines to CDK4/6 inhibitors independently of pRB, PIK3CA, PTEN and BRCA1 mutations.

Using a panel of three TNBC cell lines (MDA-MB-231, MDA-MB-436 and MDA-MB-468), Example 6 demonstrates that RANK pathway mediates resistance to palbociclib, ribociclib and abemaciclib, since osteoprotegerin (OPG-Fc) restored sensitivity to therapy. Next, it was questioned if either resistance to CDK4/6 or effect of OPG-Fc could be dependent on mutational status of genes frequently mutated in TNBC, like pRB, PIK3CA, PTEN and BRCA1. Therefore, the previous analysis was expanded to include three additional TNBC cell lines (MDA-MB-157, HCC1937 and BT-20) (Table 1).

TABLE 1
Mutational profile of TNBC cell lines used in this study.

| | | | | PI3K | |
|---|---|---|---|---|---|
| Cell Line | pRb | P53 | BRCA1 | Pathway | Other |
| MDA-MB-231 | WT | Mut | WT | KRAS Mut | |
| MDA-MB-468 | Mut | Mut | WT | PTEN | Amplified |
| | | | | deletion | EGFR |
| MDA-MB-436 | Mut | Mut | Mut | WT | — |
| MDA-MB-157 | WT | Mut | WT | WT | — |
| BT-20 | WT | Mut | WT | PIK3CA | Amplified |
| | | | | mutation | EGFR |
| HCC1937 | Mut | Mut | Mut | PTEN | — |
| | | | | deletion | |

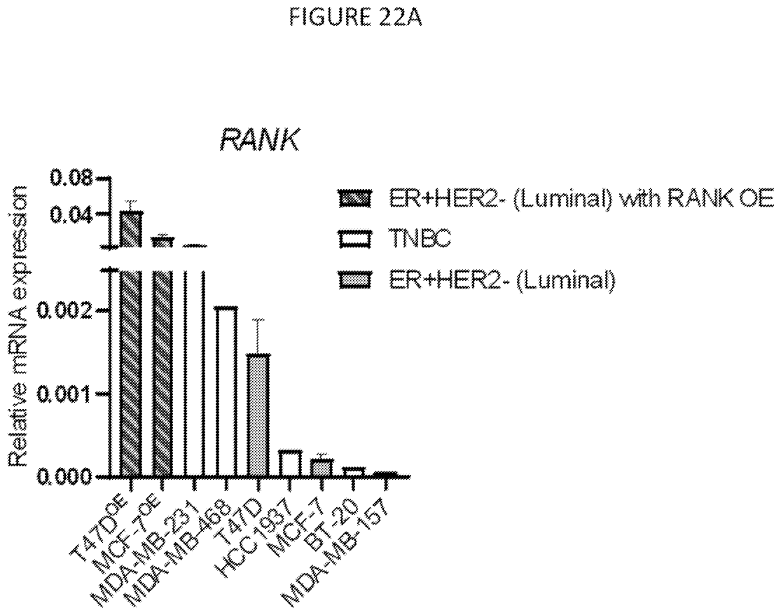

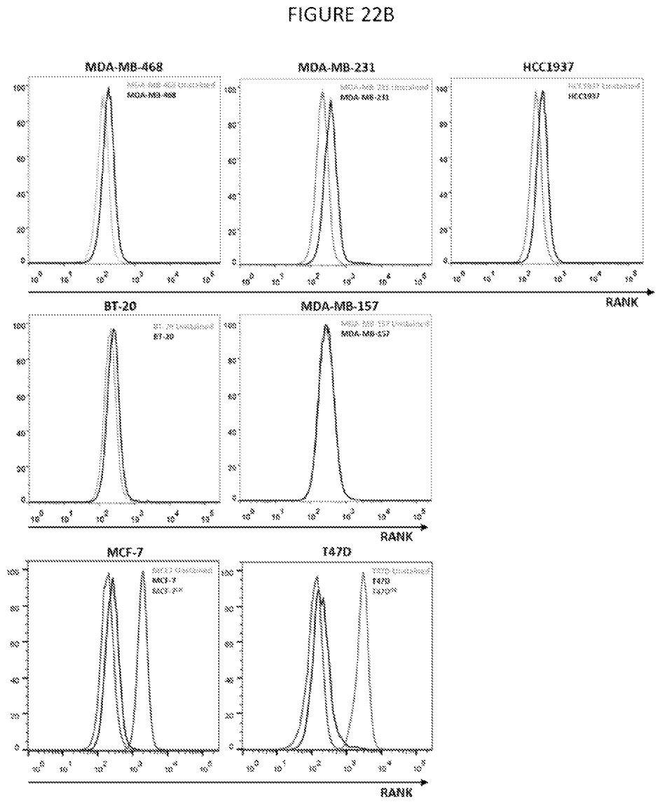

The expression of RANK in all the cell lines used in this study was assessed by RT-qPCR (FIG. 22A) and flow cytometry (FIG. 22B). RNAseq data available from the Cancer Cell Line Encyclopedia (CCLE) database is presented in Table 2. RANK expression is heterogeneous amongst TNBC and Luminal cell lines, and RANK CE luminal cell lines used in this study express the highest levels of RANK. Different methodologies used to quantify gene expression and laboratory-driven alteration of cell lines contribute to some discrepancies between data (e.g. T47D cell line).

TABLE 2
Log2RPKM value of RANK (TNFRSF11A) gene in
each breast cancer cell line used in this study based
on Cancer Cell Line Encyclopedia (CCLE) database.

| Cell line | TNFRSF11A | ER (+/−) |
|---|---|---|
| MDAMB468_BREAST | 0.904624149 | − |
| MDAMB231_BREAST | 0.838153185 | − |
| BT20_BREAST | −0.059324100 | − |
| HCC1937_BREAST | −0.950896290 | − |
| MCF7_BREAST | −1.557351949 | + |
| T47D_BREAST | −1.793258066 | + |
| MDAMB436_BREAST | −2.104042041 | − |
| MDAMB157_BREAST | −4.918962961 | − |

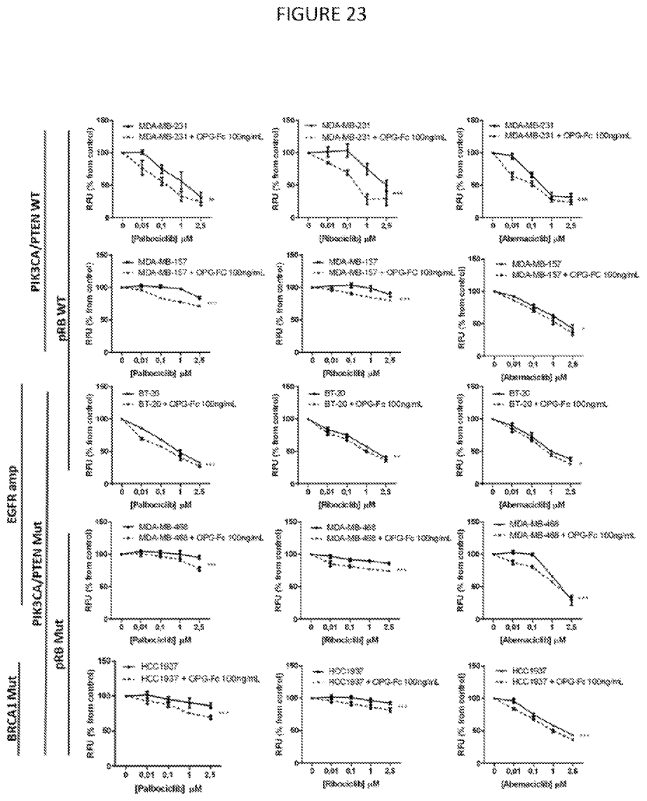

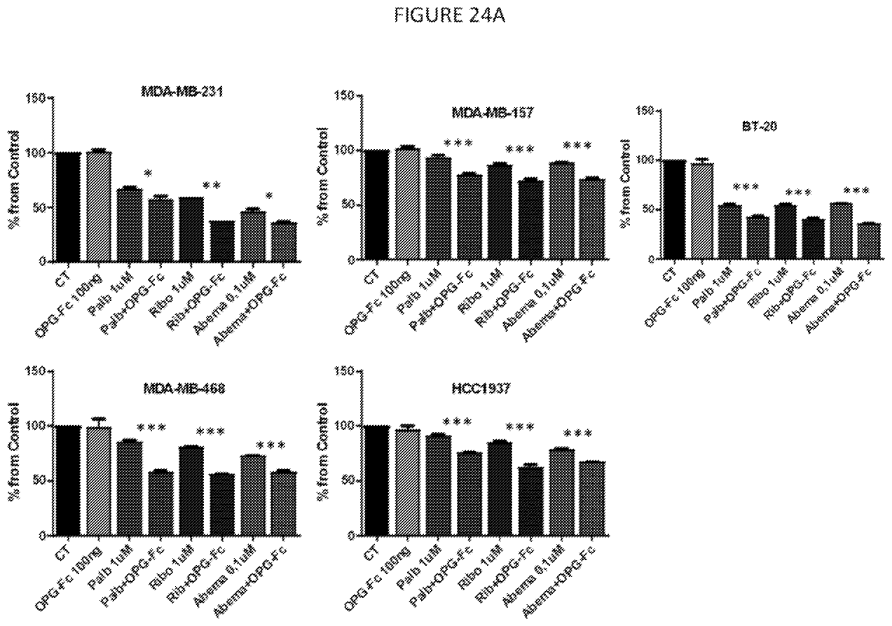

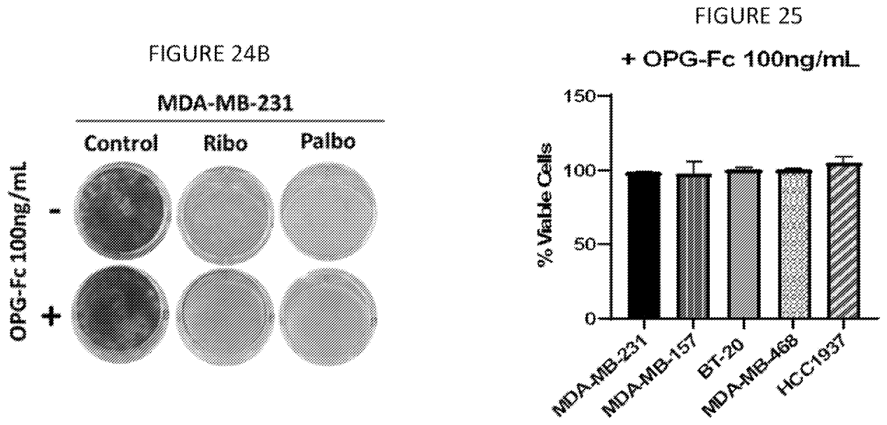

Cells were seeded in 96-well plates and exposed to different drugs for seven days. Medium was replaced every two days, and viability was assessed by Alamar blue assay. The results are shown in FIG. 23 and represent the mean of at least three independent assays, with 4 replicates per assay, and presented as the mean±SEM. p-value was calculated using 2-way ANOVA, *p<0.05, **p<0.01, ***p<0.001. These data suggest that OPG-Fc sensitizes TNBC cell lines to CDK4/6 inhibitors independently of pRB, PIK3CA, PTEN and BRCA1 mutations, and EGFR amplification (FIG. 23), as the same effect was observed in all cell lines. These findings were confirmed by clonogenic assay. Briefly, cells were seeded in 6-well plates, exposed to different drugs for six days and allowed to recover for six days in drug-free media. Cells were stained with crystal violet (FIG. 24B), lysed with 1% SDS and media absorbance measured at 570 nM (FIG. 24A). The results are shown in FIG. 24A, and images of the cell colonies are provided in FIG. 24B and the results represent the mean of 3 replicates, and presented as the mean±SEM. p-value was calculated using 2-way ANOVA, *p<0.05, **p<0.01, ***p<0.001.

### Example 20

This example demonstrates that OPG-Fc alone has no effect on proliferation of cell lines.

Cells were seeded in 96-well plates and exposed to OPG-Fc for seven days. Medium was replaced every two days, and viability was assessed by Alamar blue assay. The results are shown in FIG. 25 and represent the mean of at least three independent assays, with 4 replicates per assay, and presented as the mean±SEM. p-value was calculated using 1-way ANOVA and considered non-significant if p>0.05. This suggests that the increase in CDK4/6 efficacy observed with the addition of OPG-Fc is not related to a direct effect of OPG-Fc or RANK-pathway on cell proliferation.

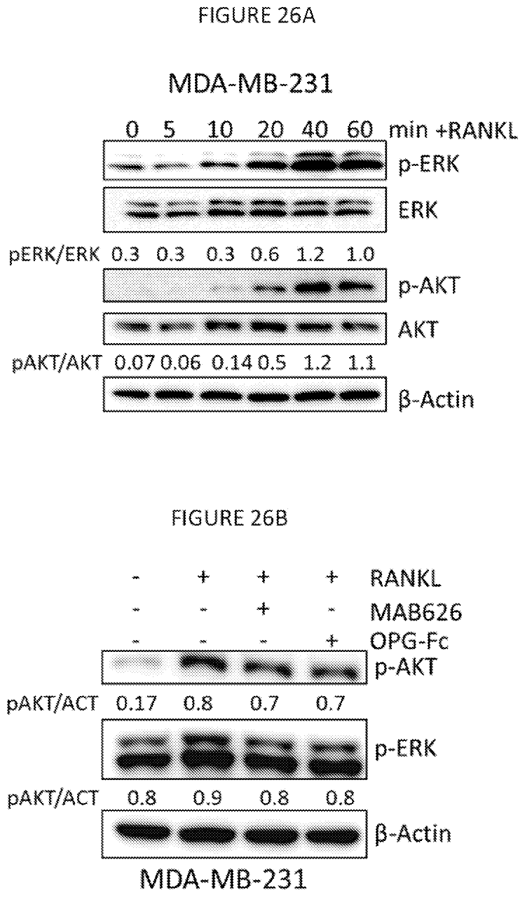

It was also confirmed that OPG-Fc is able to neutralize RANKL. Protein expression was assessed by western blot. Cells were seeded in 6-well plates, serum starved for 24 h, and exposed to 1 μg/ml RANKL for the indicated time points (FIG. 26A). For RANKL neutralization (FIG. 26B), RANKL was previously incubated for 60 min at 37° C. in serum-depleted medium±100 ng/ml OPG-Fc or 2.5 μg/ml MAB626, and proteins analyzed after 60 min. β-Actin was used as loading control and band intensity was quantified using FiJi. RANKL stimuli drives the phosphorylation of RANK-downstream factors, like ERK and AKT (FIG. 26A). In the presence of OPG-Fc or the anti-RANKL monoclonal antibody MAB626, RANKL-driven RANK pathway activation was decreased (FIG. 26B).

### Example 21

This example demonstrates TNBC RANK-mediated resistance to CDK4/6 inhibitors and demonstrates OPG-Fc decreases tumor growth in vivo.

Data provided herein demonstrate that ER+HER2− RANK OE cells were characterized by decreased proliferation rate but increased invasiveness in vitro and in vivo (Gomes et al. 2020 in Revision). MCF-7 RANK OE cells were shown as resistant to palbociclib in vivo, in an orthotopic model. An experiment showing that RANK pathway inhibition (OPG-Fc) sensitizes TNBC cells to CDK4/6 inhibitors (palbociclib) in vivo was carried out (Example 10), as shown by a significant decrease in Ki67 and p-pRB expression in tumors treated with palbociclib plus OPG-Fc.

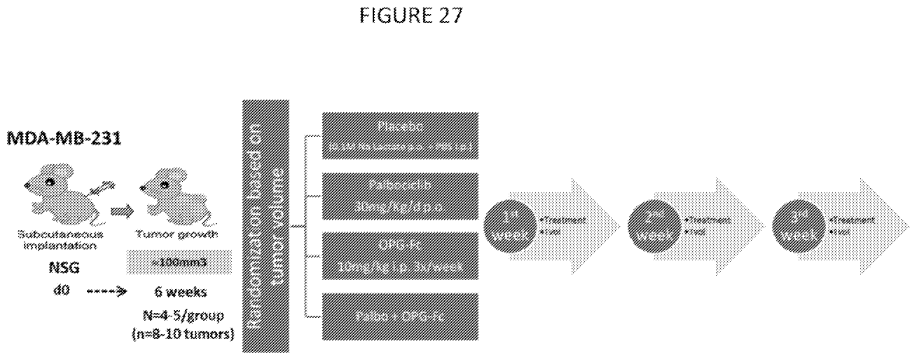

Based on these findings TNBC BC sensitivity to CDK4/6 inhibitors (palbociclib) in combination with RANK pathway blockage was tested in a TNBC ectopicxenograft model (FIG. 27). Nod scid gama (NSG) mice were inoculated subcutaneously and bilaterally in the flanks (n=5/group). Tumors were measured with caliper. When tumors reach approximately 100 mm³ mice were randomized based on tumor size and treated according to the experimental protocol. Palbociclib was used at 30 mg/Kg/day for 21 consecutive days, as the human equivalent dose (HED) of 125 mg/day approved for palbociclib is equivalent to 29.25 mg/Kg/day in mice (Nair and Jacob, J Basic Clin Pharm. 2016 March; 7(2):27-31). OPG-Fc was used at 10 mg/Kg 3×/week (HED 291.6 mg/15 days).

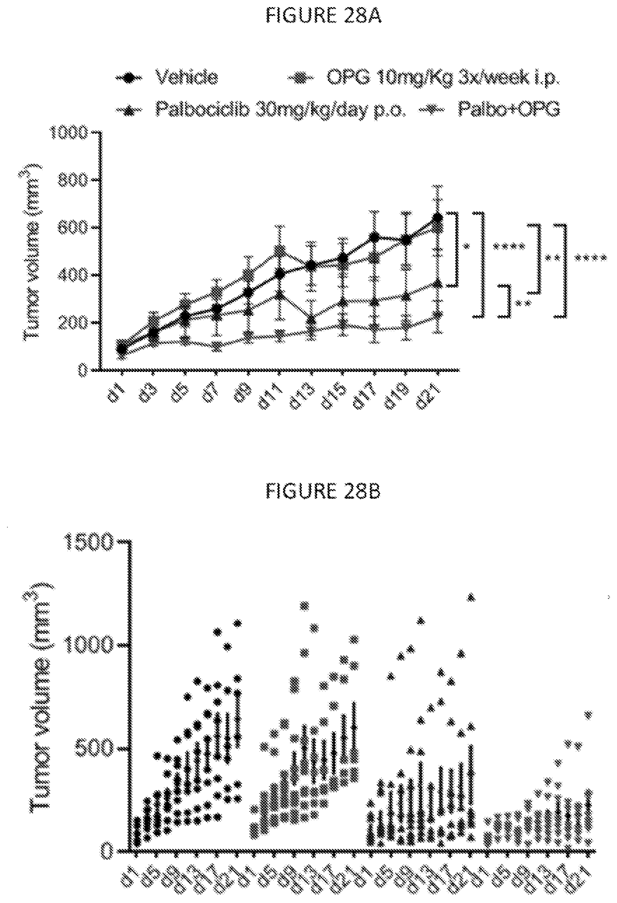

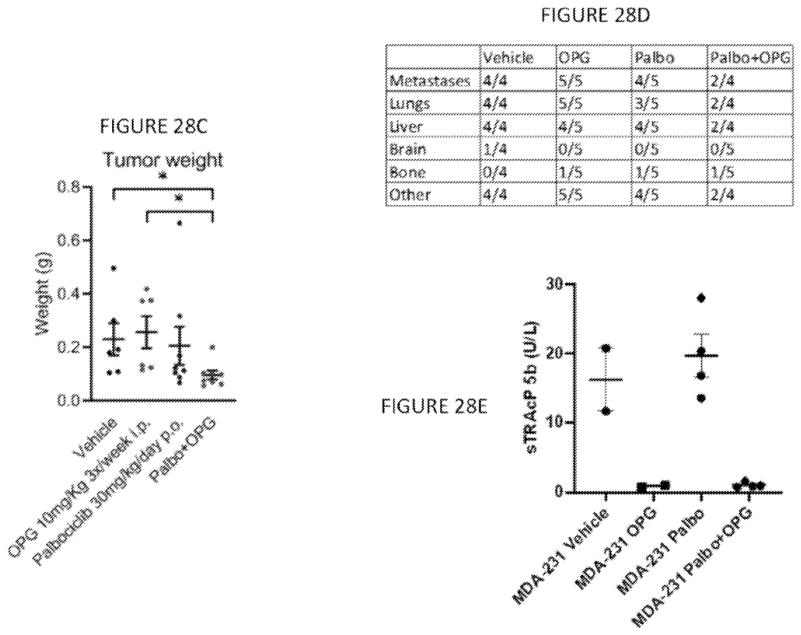

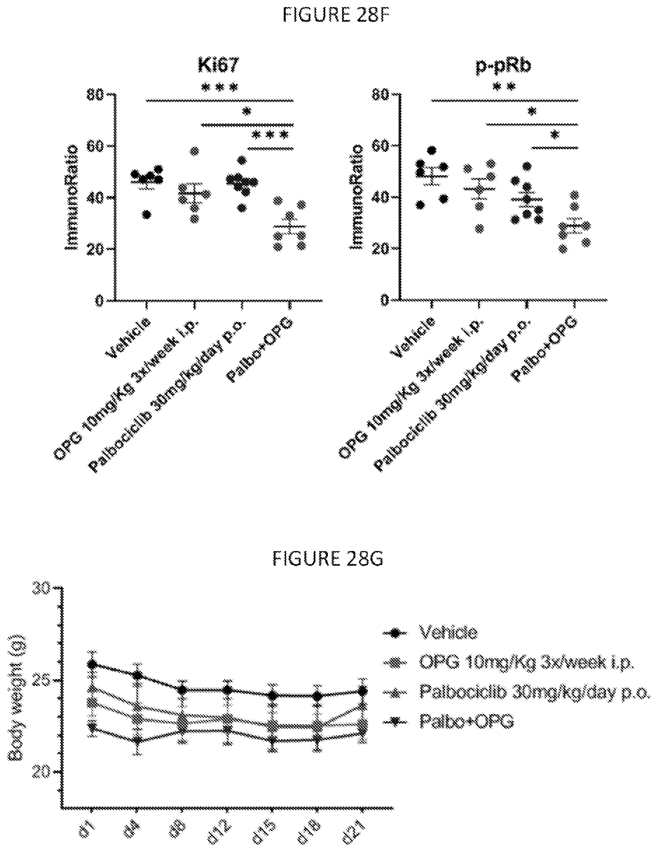

Nod scid gama (NSG) mice were inoculated subcutaneously and bilaterally in the flanks with MDA-MB-231 cells (n=4-5/group). Six weeks post inoculation mice were randomized based on tumor size and treated with OPG-Fc 10 mg/Kg i.p. 3× per week; Palbociclib 30 mg/Kg/day p.o. or the combination. Tumor volume was measured with caliper every two days and calculated using the formula Tᵥₒₗ=½(length×width² and graphs of tumor volume plotted as a function of time are provided in FIGS. 28A and 28B. Tumor weight at necropsy for each group of mice is provided in the graph of FIG. 28C. The number of mice with metastases after histopathological assessment of organs post necropsy is provided in the table of FIG. 28D. Osteoclast-specific TRAcP 5b was quantified in serum collected at necropsy and a graph of the sTRAcP 5B is provided in FIG. 28E. Quantification of Ki67 and p-pRb (ImunoRatio) is provided in the pair of graphs in FIG. 28F. A graph of the body weight of mice plotted as a function of time is provided in FIG. 28G. The data are presented as mean±SEM. p-value was calculated using ANOVA, *p<0.05, **p<0.01, ***p<0.001.

MDA-MB-231 xenografts were not regressed on therapy, but showed slowing of tumor growth after CDK4/6 inhibitor treatment with palbociclib, which was improved by OPG-Fc (FIG. 28A,B). Accordingly, Ki67 and p-pRb were significantly decreased in tumors from mice treated with the combination (FIG. 28F). OPG-Fc suppressed osteoclast activity as confirmed assessed by serum TRAcP 5b quantification (FIG. 28E).

### Example 22

This example demonstrates additional studies to test the efficacy of a combination treatment comprising a RANK pathway inhibitor and a CDK4/6 inhibitor.

An in vivo model to test the efficacy of Palbociclib+Fulvestrant+OPG-Fc in comparison to Palbociclib+Fulvestrant in RANK OE luminal breast cancer cells is made. Nod scid gamma (NSG) mice are inoculated subcutaneously and bilaterally in the flanks (n=5/group). Tumors are measured with calipers. When tumors reach approximately 100 mm³ mice are randomized based on tumor size and treated according to the experimental protocol (FIG. 29).

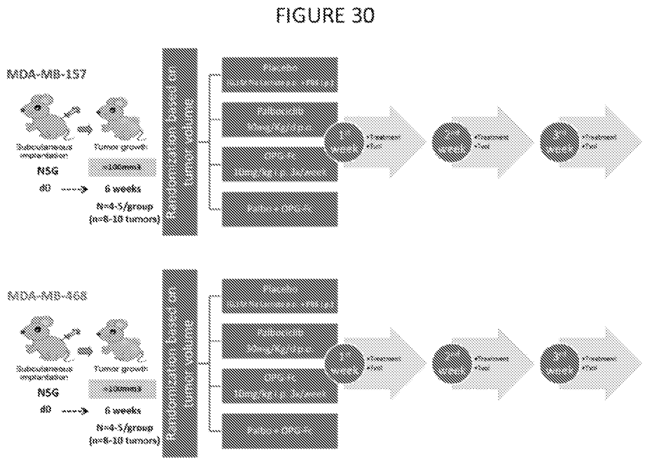

Also, an in vivo model to expand the tests on the efficacy of Palbociclib+OPG-Fc in comparison with Palbociclib alone in TNBC cells (MDA-MB-157 and MDA-MB-468) is made. By choosing these two cell lines it is intended to test cells with higher Palbociclib IC50 than MDA-MB-231, and with different RANK expression; as well as a pRB deficient cell line (MDA-MB-468). Nod scid gama (NSG) mice are inoculated subcutaneously and bilaterally in the flanks (n=5/group). Tumors are measured with calipers. When tumors reach approximately 100 mm³ mice are randomized based on tumor size and treated according to the experimental protocol shown in FIG. 30.

Upon the conclusion of these experiments, tumors will be analyzed by deep coverage RNA-seq, to elucidate on the molecular mechanism of resistance and OPG-Fc effect. Findings will be functionally validated (e.g. in cell lines, patient-derived organoids (PDO), patient-derived xenografts (PDX), and stable cell lines from PDX).

### Example 23

This example describes the materials and methods used in Examples 17-22.

Cell Culture

BT-20 and MDA-MB-157 cells were purchased from ATCC. Human breast carcinoma cell lines MDA-MB-231GFP+Luc+, and MCF-7GFP+Luc+ (herein designated by MDA-MB-231 and MCF-7) were provided by Sérgio Dias Lab (IMM), and derived from parental cells by lentiviral transduction with GFP-Luciferase lentiviral particles and cell sorting of pure GFP+ cell populations. T47D cells were provided by Philippe Clézardin Lab at INSERM. MDA-MB-468 and HCC1937 were provided by Rita Fior Lab at Fundação Champalimaud. MDA-MB-231 and MDA-MB-468 cells were cultured in Dulbecco's Modified Eagle's Medium (DMEM, Gibco), BT-20 in Modified Eagle's Medium (MEM, Gibco), and HCC1937 in RPMI 1640 (Gibco), all supplemented with 10% (v/v) fetal bovine serum (FBS, Gibco) (20% for MDA-MB-157), 1% (v/v) Penicillin/Streptomycin (10,000 U/mL Penicillin, 10,000 μg/mL Streptomycin, Gibco). MCF-7 and T47D cells were cultured in the same medium, additionally supplemented with 0.01 mg/mL insulin (Gibco). Cells were maintained at 37° C. with 5% C02, used at low passage number, and tested for Mycoplasma contamination by qPCR.

RT-qPCR

Cells total RNA was extracted using the NZY Total RNA Isolation kit (#MB13402, Nzytech). DNase I-treated RNA was reverse transcribed using the NZY M-MuLV First-Strand cDNA Synthesis kit (#MB17301, Nzytech) and Oligo(dT)20 primer; and cDNAs were amplified by real-time PCR using TaqMan Gene Expression Master Mix (#4369016, Applied Biosystems) and specific primers for TNFRSF11A (#Hs00921372_ml, Applied Biosystems) and GAPDH (#PPH00150F, SA Biosciences). Gene expression was normalized using the housekeeping gene GAPDH, and relative mRNA expression was calculated using the 2-ACt method.

Flow Cytometry

For RANK expression analysis, trypsinized cells were incubated with mouse monoclonal antibody anti-RANK (#M331, Amgen Inc.) and labelled with 1:100 Cy5 conjugated AffiniPure goat anti-mouse IgG (#115-175-205, Dianova). Analysis was made using FlowJo V10 software.

In Silico Analysis

Normalized RANK (TNFRSF11A) expression (log 2 RPKM (Reads Per Kilobase Million)) in breast cancer cell lines was derived from the Cancer Cell Line Encyclopedia (CCLE) database (https://portals.broadinstitute.or/ccle).

Western Blot

Activation of RANK pathway upon stimuli with RANKL was analyzed by Western blot.

For this purpose, 4×10⁵ cells were seeded in 6-well plates for 24 h, and serum-starved in low-serum medium (0,1% FBS, 1% Pen/Step) for another 24 h. Medium was replaced by fresh low-serum medium containing 1 μg/mL human RANKL (Amgen) and total cell lysates obtained at different time points. For RANKL neutralization, RANKL was incubated in low-serum medium at 37° C. for 60 min with 100 ng/ml OPG-Fc (PL-33324, Amgen) or 2.5 μg/ml MAB626 (R&D). Total cell lysates were prepared with RIPA buffer containing protease and phosphatase inhibitors cocktails (1:100; Santa Cruz), according to manufacturer's instructions. Total protein was quantified using Pierce BCA Protein Assay Kit (ThermoSicentific), according to manufacturer's instructions. Proteins were resolved by SDS-PAGE, using 10% polyacrylamide gels, and then transferred to nitrocellulose membranes using an iBlot®2 Gel Transfer Device (Invitrogen), according to manufacturer's instruction.

Membranes were blocked for 1 h at room temperature (RT) in 5% Non-Fat Dry Milk (NFDM) in PBS-0.1% Tween (PBST) for β-actin; or in 5% bovine serum albumin (BSA) (Santa Cruz) for other antibodies. Membranes were incubated with the following specific antibodies, overnight at 4° C.: mouse anti-β Actin antibody (Ab6276; Abcam), rabbit polyclonal anti Phospho-ERK1/2 (Thr-202/Tyr-204) (#sc-1682, Santa Cruz Biotechnology), rabbit polyclonal anti ERK1/2 (c-14) (#sc-154, Santa Cruz Biotechnology), rabbit polyclonal anti Phospho-AKT1/2/3 (Ser-473) (D9E) (#sc-7985, Santa Cruz Biotechnology), rabbit polyclonal anti AKT1/2/3 (H-136) (#sc-8312, Santa Cruz Biotechnology), After washing with PBST, membranes were incubated with horseradish peroxidase-conjugated (HRP) specific secondary antibodies: anti-mouse-HRP IgG and anti-rabbit-HRP IgG (1:5000; Cell Signaling), for 2 h at RT. Proteins were detected using a Novex® ECL HRP chemiluminescent substrate reagent kit (Invitrogen) according to the manufacturer's instructions, and x-ray films (Fujifilm) developed in a Curix 60 processor (AGFA), or the Amersham™ Imager 680 (GE Healthcare Life Sciences).

Viability Assays

Cells were seeded in 96 well-plates (2-5×10⁴ cells/mL), with or without palbociclib (PD 0332991 isethionate, PZ0199, Sigma), ribociclib (LEE011 succinate, SC-488174, Santa Cruz), abemaciclib mesylate (LY2835219, S7158, Selleckchem), 3-ATA (sc-202414, Santa Cruz), Cdk4 inhibitor Ill (sc-202988, Santa Cruz), seliciclib (S1153, Selleckchem), and OPG-Fc (PL-33324, Amgen). Medium was replaced every 2 days. After 7 days, 1:10 Alamar blue (Invitrogen) was added to each well and fluorescence was measured 2 hours after incubation (excitation 560 nm; emission 590 nm) in an Infinite M200 microplate reader (Tecan).

Clonogenic Assays

For colony formation assay, 3,000 to 5,000 cells/well were plated in 6-well plates, treated for 6 days with test drugs, and allowed to recover for another six days in drug-free medium. Cells were fixed with 2% PFA for 10 min, and stained with 1% crystal violet solution (Sigma). Stain was solubilized in 1% SDS, and absorbance measured at 570 nm in an Infinite M200 microplate reader (Tecan).

Animal Model

All animal experiments were reviewed and approved by Institutional Animal Welfare Body, and licensed by the Direcgso Geral de Alimentação e Veterinaria (DGAV). In all studies involving animals, mice were handled and euthanized in accordance with approved institutional, national and international guidelines, applying the Principle of the 3Rs.

Ectopic Xenograft Model

For the ectopic MDA-MB-231 xenograft' model, cells were harvested at the exponential phase of growth and resuspended at 1.0×10⁶ cells/ml in 50% phenol-free matrigel solution (Corning). Four-week-old NOD scid gamma (NSG) mice (Charles River) mice were injected bilaterally with 100 μl of cell suspension, directly into the flanks. Tumor growth was monitored every two days with caliper and tumor volume calculated using the formula Tvol=½(length×width²). When tumors reached 100 mm³, mice were randomized based on tumor size and treated with palbociclib 30 mg/Kg/d p.o.; OPG-Fc 10 mg/kg i.p. 3×/wek; Palbociclib+OPG-Fc 10 mg/Kg; or vehicle, for 21 consecutive days. Mice were sacrificed by administration of 0.25 mg/KgBW Sodium Pentobarbital (Eutasil). At necropsy, primary tumors were harvested and sectioned into two fragments for paraffin embedding or snapshot freezing. Organs were harvested and paraffin embedded.

Immunohistochemistry

5 μm tissue sections from FFPE samples were stained by immunohistochemistry (IHC) for the detection of Ki67 and p-pRb (Ser807/11). Deparaffinization and antigen retrieval was performed in a PT Link Pre-Treatment Module for Tissue Specimens (Dako), using Antigen Retrieval Solution pH9.0, at 94° C. for 20 min. Endogenous peroxidase was blocked with Preoxidase Blocking Solution (Dako) for 10 min at RT, and total protein was blocked by incubation with Protein Block Solution (Dako), for 20 min at RT. Slides were incubated for 30 min with rabbit anti-human Ki67 primary antibody (1:100, MIB-1, Dako) or rabbit monoclonal anti Phospho-Rb (Ser807/811) (D20B12) (1:400, #8516, Cell Signaling), in Antibody Diluent (Dako). Slides were incubated with EnVision™ Detection System, rabbit/mouse (Dako), according to manufacturer's instructions, followed by 5 min of incubation with DAB (Dako). Slides were counterstained with hematoxylin, dehydrated, mounted with Quick-D mounting medium (Klinipath) and visualized in a bright field microscope (LeicaDM750 with a Leica ICC50 HD camera). Imunoratio was obtained through the calculi of the percentage of DAB-stained nuclear area over total nuclear area (hematoxylin-stained nuclei regions) (5 fields, 400×, ImageJ software).

sTRAcP 5b ELISA

TRAcP 5b was quantified in mouse serum using the MouseTRAP (TRAcP 5b) ELISA kit (IDS), according to the manufacturer instructions.

Statistical Analysis

Data was analysed using GraphPad Prism6 software. The number of replicates performed for each experiment is indicated. Statistics were performed by one-way ANOVA or unpaired t-test (in vitro experiments); two-way ANOVA (tumour burden and mice BW). Results are presented as mean with SEM and p-value <0.05 was considered significant.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A pharmaceutical composition comprising i) a RANK pathway inhibitor in combination with ii) a CDK inhibitor.

2. The pharmaceutical composition of claim 1, wherein the RANK pathway inhibitor inhibits a binding interaction between RANK and RANK ligand (RANKL).

3. The pharmaceutical composition of claim 2, wherein the RANK pathway inhibitor comprises osteoprotegerin (OPG), a RANKL-binding fragment thereof, or an antigen-binding protein that binds to RANK or RANKL.

4. The pharmaceutical composition of claim 3, wherein the antigen-binding protein is a fully human antibody, a humanized antibody, or a chimeric antibody.

5. The pharmaceutical composition of claim 3, wherein the antigen-binding protein is a Fab, Fab′, F(ab′)2, or a single chain Fv comprising one, two, three, four, five or more of the heavy and light chain complementarity determining region (CDR) of an anti-RANK antibody or an anti-RANKL antibody.

6. The pharmaceutical composition of any one of claims 3 to 5, wherein the antigen-binding protein binds to RANKL.

7. The pharmaceutical composition of claim 6, wherein the antigen-binding protein comprises:
a. a heavy chain CDR1 amino acid sequence of SEQ ID NO: 8, or a variant sequence thereof which differs by only one or two amino acids or which has at least or about 70% sequence identity;
b. a heavy chain CDR2 amino acid sequence of SEQ ID NO: 9, or a variant sequence thereof which differs by only one or two amino acids or which has at least or about 70% sequence identity;
c. a heavy chain CDR3 amino acid sequence of SEQ ID NO: 10, or a variant sequence thereof which differs by only one or two amino acids or which has at least or about 70% sequence identity;
d. a light chain CDR1 amino acid sequence of SEQ ID NO: 5, or a variant sequence thereof which differs by only one or two amino acids or which has at least or about 70% sequence identity;
e. a light chain CDR2 amino acid sequence of SEQ ID NO: 6, or a variant sequence thereof which differs by only one or two amino acids or which has at least or about 70% sequence identity;
f. a light chain CDR3 amino acid sequence of SEQ ID NO: 7, or a variant sequence thereof which differs by only one or two amino acids or which has at least or about 70% sequence identity; or
g. a combination of any two or more of (a)-(f).

8. The pharmaceutical composition of claim 7, wherein the antigen-binding protein comprises
(A) a light chain variable domain selected from the group consisting of:
(i). a light chain variable domain comprising an amino acid sequence or SEQ ID NO: 1, or a variant sequence which differs by only one or two amino acids or which has at least or about 70% sequence identity to SEQ ID NO: 1;
(ii). a light chain variable domain comprising an amino acid sequence encoded by a polynucleotide sequence comprising SEQ ID NO: 19;
(iii). a light chain variable domain comprising an amino acid sequence encoded by a polynucleotide that hybridizes under stringent conditions to the complement of a polynucleotide consisting of SEQ ID NO: 19; or
(B) a heavy chain variable domain selected from the group consisting of:
(i). a heavy chain variable domain comprising an amino acid of SEQ ID NO: 2, or a variant sequence which differs by only one or two amino acids or which has at least or about 70% sequence identity to SEQ ID NO: 2;
(ii). a heavy chain variable domain comprising an amino acid sequence encoded by a polynucleotide sequence comprising SEQ ID NO: 20.
(iii). a heavy chain variable domain comprising an amino acid sequence encoded by a polynucleotide that hybridizes under stringent conditions to the complement of a polynucleotide consisting of SEQ ID NO: 20; or
(C) a light chain variable domain of (A) and a heavy chain variable domain of (B).

9. The pharmaceutical composition of claim 7 or 8, wherein the variant sequence has at least or about 80%, at least or about 85%, at least or about 90%, at least or about 95% sequence identity to the SEQ ID NO.

10. The pharmaceutical composition of any one of claims 3 to 9, wherein the antigen-binding protein is an IgG1, IgG2, or IgG4 antibody, optionally, comprising a kappa light chain.

11. The pharmaceutical composition of any one of claims 3 to 10, wherein the antigen-binding protein comprises the amino acid sequence of SEQ ID NO: 15.

12. The pharmaceutical composition of any one of claims 3 to 11, wherein the antigen-binding protein comprises the amino acid sequence of SEQ ID NO: 16, SEQ ID NO: 17, or SEQ ID NO: 18.

13. The pharmaceutical composition of any one of claims 3 to 12, wherein the antigen-binding protein comprises:
(A) a light chain selected from the group consisting of:
(i). a light chain comprising an amino acid sequence of SEQ ID NO: 3 or SEQ ID NO: 13 or a variant sequence which differs by only one or two amino acids or which has at least or about 70% sequence identity to SEQ ID NO: 3 or 13;
(ii). a light chain variable domain comprising an amino acid sequence encoded by a polynucleotide sequence comprising SEQ ID NO: 21 or 23;
(iii). a light chain comprising an amino acid sequence encoded by a polynucleotide that hybridizes under stringent conditions to the complement of a polynucleotide consisting of SEQ ID NO: 21 or 23; or
(B) a heavy chain selected from the group consisting of:
(i). a heavy chain comprising an amino acid sequence of SEQ ID NO: 4 or SEQ ID NO: 14 or a variant sequence which differs by only one or two amino acids or which has at least or about 70% sequence identity to SEQ ID NO: 4 or SEQ ID NO: 14;
(ii). a heavy chain variable domain comprising an amino acid sequence encoded by a polynucleotide sequence comprising SEQ ID NO: 22 or 24.
(iii). a heavy chain comprising an amino acid sequence encoded by a polynucleotide that hybridizes under stringent conditions to the complement of a polynucleotide consisting of SEQ ID NO: 22 or 24; or
(C) a light chain variable domain of (A) and a heavy chain variable domain of (B).

14. The pharmaceutical composition of any one of claims 3 to 13, wherein the antigen-binding protein comprises the amino acid sequences of SEQ ID NOs: 5, 6, 7, 8, 9, and 10.

15. The pharmaceutical composition of any one of claims 3 to 14, wherein the antigen-binding protein comprises an amino acid sequence SEQ ID NO: 1 and an amino acid sequence of SEQ ID NO: 2.

16. The pharmaceutical composition of claim 15, wherein the antigen-binding protein further comprises an amino acid sequence of SEQ ID NO: 16 and an amino acid sequence of SEQ ID NO: 28.

17. The pharmaceutical composition of any one of claims 3 to 16, wherein the antigen-binding protein comprises an amino acid sequence of SEQ ID NO:13 and an amino acid sequence of SEQ ID NO: 14.

18. The pharmaceutical composition of any one of claims 3 to 17, wherein the antigen-binding protein comprises an amino acid sequence of SEQ ID NO:3 and an amino acid sequence of SEQ ID NO: 4.

19. The pharmaceutical composition of any one of the preceding claims, wherein the CDK inhibitor is a CDK4/6 inhibitor, optionally, a serine/threonine kinase inhibitor, a Cytochrome P450 (CYP450) 3A Inhibitor, or both

20. The pharmaceutical composition of claim 19, wherein the CDK4/6 inhibitor inhibits the phosphorylation of retinoblastoma (Rb) protein.

21. The pharmaceutical composition of claim 19 or 20, wherein the CDK4/6 inhibitor comprises a structure of Structure I or Structure II:

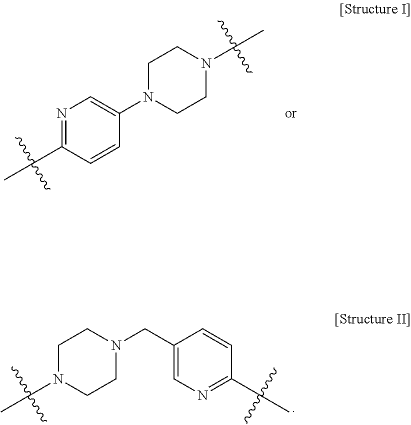 [SMILES: *c1ccc(N2CCN(*)CC2)cn1 ; *c1ccc(CN2CCN(*)CC2)cn1]

22. The pharmaceutical composition of claim 21, wherein the CDK4/6 inhibitor comprises a structure of Structure I or Structure and further comprises a structure of A-B, wherein A comprises a bicyclic structure and B comprises a monocyclic structure.

23. The pharmaceutical composition of claim 22, wherein A-B comprises a structure of Structure III or Structure IV or Structure V:

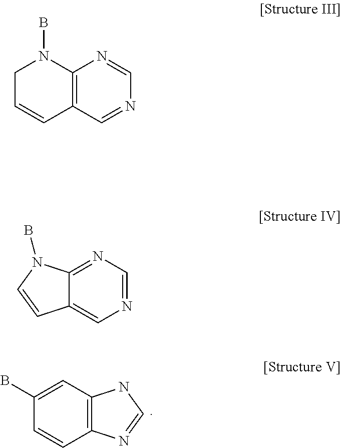

24. The pharmaceutical composition of claim 23, wherein B of Structure III or IV is a cyclopentane.

25. The pharmaceutical composition of claim 23, wherein B of Structure V comprises a pyrimidine.

26. The pharmaceutical composition of any one of claims 21 to 25, wherein the CDK4/6 inhibitor comprises the structure of 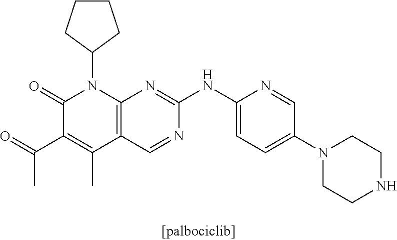 [SMILES: CC(=O)c1c(C)c2cnc(Nc3ccc(N4CCNCC4)cn3)nc2n(C2CCCC2)c1=O]
or a pharmaceutically acceptable salt thereof.

27. The pharmaceutical composition of any one of claims 21 to 25, wherein the CDK4/6 inhibitor comprises the structure of 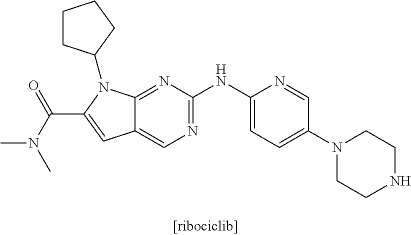 [SMILES: CN(C)C(=O)c1cc2cnc(Nc3ccc(N4CCNCC4)cn3)nc2n1C1CCCC1]
or a pharmaceutically acceptable salt thereof.

28. The pharmaceutical composition of any one of claims 21 to 25, wherein the CDK4/6 inhibitor comprises the structure of 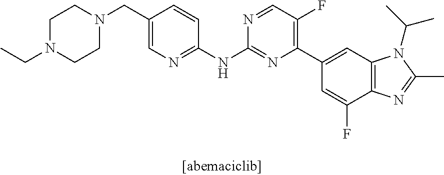 [SMILES: CCN1CCN(Cc2ccc(Nc3ncc(F)c(-c4cc(F)c5nc(C)n(C(C)C)c5c4)n3)nc2)CC1]
or a pharmaceutically acceptable salt thereof.

29. The pharmaceutical composition of any one of the preceding claims, wherein the CDK inhibitor is packaged separately from the RANK pathway inhibitor.

30. The pharmaceutical composition of any one of the preceding claims, further comprising a hormone therapy agent.

31. The pharmaceutical composition of any one of the preceding claims, further comprising an aromatase inhibitor, an ER-targeted agent, rapamycin or a rapamycin analog, an anti-HER2 drug or a PI3K inhibitor.

32. The pharmaceutical composition of claim 31, wherein the aromatase inhibitor is letrozole, anastrozole, or exemestante.

33. The pharmaceutical composition of claim 31, wherein the rapamycin analog is everolimus, temsirolimus, ridaforolimus, zotarolimus, and 32-deoxo-rapamycin.

34. The pharmaceutical composition of claim 31, wherein the ER-targeted agent is fulvestrant or tamoxifen.

35. The pharmaceutical composition of claim 31, wherein the anti-HER2 drug is trastuzumab, pertuzumab, lapatinib, T-DM1, or neratinib.

36. The pharmaceutical composition of claim 31, wherein the PI3K inhibitor is taselisib, alpelisib or buparlisib.

37. The pharmaceutical composition of any one of the preceding claims, which (A) increases or restores responsiveness or sensitivity of a cancer cell to treatment with a CDK inhibitor, (B) treats a subject with a cancer, optionally, wherein the cancer exhibits a reduced responsiveness to treatment with a CDK inhibitor or the subject is or has been treated with a CDK inhibitor, (C) delays the occurrence or onset of metastasis in a subject, (D) reduces tumor growth or tumor burden or increases tumor regression in a subject, optionally, wherein the subject is or has been treated with a CDK inhibitor, (E) increases progression-free survival, overall survival, or time to deterioration of Eastern Cooperative Oncology Group (ECOG) performance status in a subject with a cancer, optionally, wherein the cancer is resistant to or exhibits a reduced sensitivity to a CDK inhibitor, (F) reduces the level of circulating tumor cells (CTCs) in a subject, or (G) any combination thereof.

38. A pharmaceutical composition of any one of the preceding claims for use in increasing or restoring responsiveness or sensitivity of a cancer cell to treatment with a CDK inhibitor, treating cancer in a subject, delaying the occurrence or onset of metastasis in a subject with cancer, reducing tumor growth or tumor burden or increasing tumor regression in a subject, increasing progression-free survival, overall survival, or time to deterioration of Eastern Cooperative Oncology Group (ECOG) performance status in a subject with a tumor or cancer cell resistant to or with a reduced sensitivity to a CDK inhibitor, and/or reducing the level of circulating tumor cells (CTCs) in a subject.

39. A method of increasing or restoring responsiveness or sensitivity of a cancer cell to treatment with a CDK inhibitor, comprising administering a RANK pathway inhibitor to a subject who is or has been treated with a CDK inhibitor.

40. A method of increasing or restoring responsiveness or sensitivity of a cancer cell to treatment with a CDK inhibitor, comprising administering to the subject a RANK pathway inhibitor optionally in combination with a CDK inhibitor.

41. A method of treating cancer in a subject who is or has been treated with a CDK inhibitor, comprising administering to the subject a RANK pathway inhibitor optionally in combination with the CDK inhibitor.

42. A method of treating a subject with a cancer with a reduced responsiveness to treatment with a CDK inhibitor, comprising administering to the subject a RANK pathway inhibitor optionally in combination with the CDK inhibitor.

43. A method of treating a subject with a cancer, wherein (i) cells of the cancer overexpress one or more of RANK, CDK 4, CDK 6, or Cyclin D, (ii) the subject has an increased level of circulating tumor cells (CTCs), or (iii) a combination thereof, said method comprising administering to the subject a RANK pathway inhibitor optionally in combination with the CDK inhibitor.

44. A method of delaying the occurrence or onset of metastasis in a subject with cancer, wherein the subject is or has been treated with a CDK inhibitor, comprising administering a RANK pathway inhibitor to the subject.

45. A method of delaying the occurrence or onset of metastasis in a subject with cancer, comprising administering a RANK pathway inhibitor to the subject optionally in combination with a CDK inhibitor.

46. A method of reducing tumor growth or tumor burden or increasing tumor regression in a subject who is or has been treated with a CDK inhibitor, comprising administering to the subject a RANK pathway inhibitor.

47. A method of reducing tumor growth or tumor burden or increasing tumor regression in a subject, comprising administering to the subject a RANK pathway inhibitor optionally in combination with a CDK inhibitor.

48. A method of increasing progression-free survival, overall survival, or time to deterioration of Eastern Cooperative Oncology Group (ECOG) performance status in a subject with a tumor or cancer cell resistant to or with a reduced sensitivity to a CDK inhibitor, comprising administering to the subject a RANK pathway inhibitor optionally in combination with a CDK inhibitor.

49. A method of reducing the level of circulating tumor cells (CTCs) in a subject, comprising administering to the subject a RANK pathway inhibitor optionally in combination with a CDK inhibitor.

50. The pharmaceutical composition for use of claim 38 or method of any one of claims 39 to 49, wherein the RANK pathway inhibitor and the CDK inhibitor are administered separately.

51. The pharmaceutical composition for use of claim 38 or method of any one of claims 39 to 49, wherein the RANK pathway inhibitor and the CDK inhibitor are simultaneously administered to the subject.

52. The pharmaceutical composition for use of claim 38 or method of any one of claims 39 to 51, wherein the RANK pathway inhibitor is administered to the subject via subcutaneous injection.

53. The pharmaceutical composition for use of claim 38 or method of any one of claims 39 to 52, wherein the CDK inhibitor is administered orally to the subject.

54. The pharmaceutical composition for use of claim 38 or method of any one of claims 39 to 52, wherein the RANK pathway inhibitor is administered to the subject once every 2 to 6 weeks, optionally, once every 4 weeks.

55. The method of any one of claims 38 to 53, wherein the RANK pathway inhibitor is administered to the subject once every 2 to 8 months, optionally, once every 6 months.

56. The method of any one of claims 38 to 54, wherein the CDK inhibitor is administered once daily to the subject.

57. The method of any one of claims 38 to 55, wherein subject has cancer and the cancer comprises cells that express RANK or RANK-L.

58. The method of any one of claims 38 to 56, wherein the subject has a cancer with a metastasis, an unresectable tumor, or a combination thereof.

59. The method of any one of claims 38 to 57, wherein the subject has breast cancer.

60. The method of claim 58, wherein the breast cancer is triple negative breast cancer.

61. The method of claim 58 or 59, wherein the breast cancer is hormone receptor (HR)-positive, HER2-negative.

62. The method of any one of claims 58 to 60, wherein the breast cancer is advanced breast cancer and/or metastatic breast cancer.

63. The method of any one of claims 38 to 61, wherein the subject exhibits or has exhibited a resistance or reduced sensitivity to treatment with a CDK inhibitor.

64. The method of any one of claims 38 to 62, wherein the RANK pathway inhibitor inhibits a binding interaction between RANK and RANK ligand (RANKL).

65. The method of claim 63, wherein the RANK pathway inhibitor comprises osteoprotegerin (OPG), a RANKL-binding fragment thereof, or an antigen-binding protein that binds to RANK or RANKL.

66. The method of claim 64, wherein the antigen-binding protein is a fully human antibody, a humanized antibody, or a chimeric antibody.

67. The method of claim 64, wherein the antigen-binding protein is a Fab, Fab′, F(ab′)2, or a single chain Fv comprising one, two, three, four, five or more of the heavy and light chain complementarity determining region (CDR) of an anti-RANK antibody or an anti-RANKL antibody.

68. The method of any one of claims 64 to 66, wherein the antigen-binding protein binds to RANKL.

69. The method of claim 67, wherein the antigen-binding protein comprises:
a. a heavy chain CDR1 amino acid sequence of SEQ ID NO: 8, or a variant sequence thereof which differs by only one or two amino acids or which has at least or about 70% sequence identity;
b. a heavy chain CDR2 amino acid sequence of SEQ ID NO: 9, or a variant sequence thereof which differs by only one or two amino acids or which has at least or about 70% sequence identity;
c. a heavy chain CDR3 amino acid sequence of SEQ ID NO: 10, or a variant sequence thereof which differs by only one or two amino acids or which has at least or about 70% sequence identity;
d. a light chain CDR1 amino acid sequence of SEQ ID NO: 5, or a variant sequence thereof which differs by only one or two amino acids or which has at least or about 70% sequence identity;
e. a light chain CDR2 amino acid sequence of SEQ ID NO: 6, or a variant sequence thereof which differs by only one or two amino acids or which has at least or about 70% sequence identity;
f. a light chain CDR3 amino acid sequence of SEQ ID NO: 7, or a variant sequence thereof which differs by only one or two amino acids or which has at least or about 70% sequence identity;
g. a combination of any two or more of (a)-(f).

70. The method of claim 68, wherein the antigen-binding protein comprises
(A) a light chain variable domain selected from the group consisting of:
(i). a light chain variable domain comprising an amino acid sequence or SEQ ID NO: 1, or a variant sequence which differs by only one or two amino acids or which has at least or about 70% sequence identity to SEQ ID NO: 1;
(ii). a light chain variable domain comprising an amino acid sequence encoded by a polynucleotide sequence comprising SEQ ID NO: 19;
(iii). a light chain variable domain comprising an amino acid sequence encoded by a polynucleotide that hybridizes under stringent conditions to the complement of a polynucleotide consisting of SEQ ID NO: 19; or
(B) a heavy chain variable domain selected from the group consisting of:
(i). a heavy chain variable domain comprising an amino acid of SEQ ID NO: 2, or a variant sequence which differs by only one or two amino acids or which has at least or about 70% sequence identity to SEQ ID NO: 2;
(ii). a heavy chain variable domain comprising an amino acid sequence encoded by a polynucleotide sequence comprising SEQ ID NO: 20.
(iii). a heavy chain variable domain comprising an amino acid sequence encoded by a polynucleotide that hybridizes under stringent conditions to the complement of a polynucleotide consisting of SEQ ID NO: 20; or
(C) a light chain variable domain of (A) and a heavy chain variable domain of (B).

71. The method of claim 68 or 69, wherein the variant sequence has at least or about 80%, at least or about 85%, at least or about 90%, at least or about 95% sequence identity to the SEQ ID NO.

72. The method of any one of claims 64 to 70, wherein the antigen-binding protein is an IgG1, IgG2, or IgG4 antibody, optionally, comprising a kappa light chain.

73. The method of any one of claims 64 to 71, wherein the antigen-binding protein comprises the amino acid sequence of SEQ ID NO: 15.

74. The method of any one of claims 64 to 72, wherein the antigen-binding protein comprises the amino acid sequence of SEQ ID NO: 16, SEQ ID NO: 17, or SEQ ID NO: 18.

75. The method of any one of claims 64 to 73, wherein the antigen-binding protein comprises:
(A) a light chain selected from the group consisting of:
(i). a light chain comprising an amino acid sequence of SEQ ID NO: 3 or SEQ ID NO: 13 or a variant sequence which differs by only one or two amino acids or which has at least or about 70% sequence identity to SEQ ID NO: 3 or 13;
(ii). a light chain variable domain comprising an amino acid sequence encoded by a polynucleotide sequence comprising SEQ ID NO: 21 or 23;
(iii). a light chain comprising an amino acid sequence encoded by a polynucleotide that hybridizes under stringent conditions to the complement of a polynucleotide consisting of SEQ ID NO: 21 or 23; or
(B) a heavy chain selected from the group consisting of:
(i). a heavy chain comprising an amino acid sequence of SEQ ID NO: 4 or SEQ ID NO: 14 or a variant sequence which differs by only one or two amino acids or which has at least or about 70% sequence identity to SEQ ID NO: 4 or SEQ ID NO: 14;
(ii). a heavy chain variable domain comprising an amino acid sequence encoded by a polynucleotide sequence comprising SEQ ID NO: 22 or 24.
(iii). a heavy chain comprising an amino acid sequence encoded by a polynucleotide that hybridizes under stringent conditions to the complement of a polynucleotide consisting of SEQ ID NO: 22 or 24; or
(C) a light chain variable domain of (A) and a heavy chain variable domain of (B).

76. The method of any one of claims 64 to 74, wherein the antigen-binding protein comprises the amino acid sequences of SEQ ID NOs: 5, 6, 7, 8, 9, and 10.

77. The method of any one of claims 64 to 75, wherein the antigen-binding protein comprises an amino acid sequence SEQ ID NO: 1 and an amino acid sequence of SEQ ID NO: 2.

78. The method of claim 76, wherein the antigen-binding protein further comprises an amino acid sequence of SEQ ID NO: 16 and an amino acid sequence of SEQ ID NO: 28.

79. The method of any one of claims 64 to 77, wherein the antigen-binding protein comprises an amino acid sequence of SEQ ID NO:13 and an amino acid sequence of SEQ ID NO: 14.

80. The method of claim 78, wherein the antigen-binding protein comprises an amino acid sequence of SEQ ID NO:3 and an amino acid sequence of SEQ ID NO: 4.

81. The method of any one of the preceding claims, wherein the CDK inhibitor is a CDK4/6 inhibitor, optionally, a serine/threonine kinase inhibitor, a Cytochrome P450 (CYP450) 3A Inhibitor, or both.

82. The method of claim 80, wherein the CDK4/6 inhibitor inhibits the phosphorylation of retinoblastoma (Rb) protein.

83. The method of claim 80 or 81, wherein the CDK4/6 inhibitor comprises a structure of Structure I or Structure II:

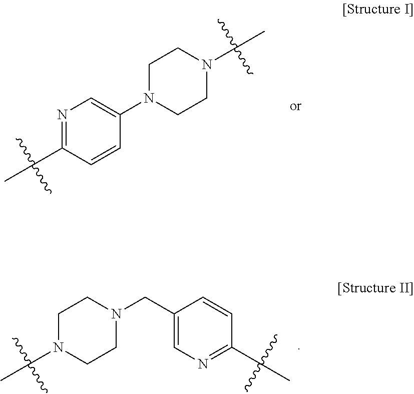 [SMILES: *c1ccc(N2CCN(*)CC2)cn1 ; *c1ccc(CN2CCN(*)CC2)cn1]

84. The method of claim 82, wherein the CDK4/6 inhibitor comprises a structure of Structure I or Structure II and further comprises a structure of A-B, wherein A comprises a bicyclic structure and B comprises a monocyclic structure.

85. The method of claim 83, wherein A-B comprises a structure of Structure III or Structure IV or Structure V:

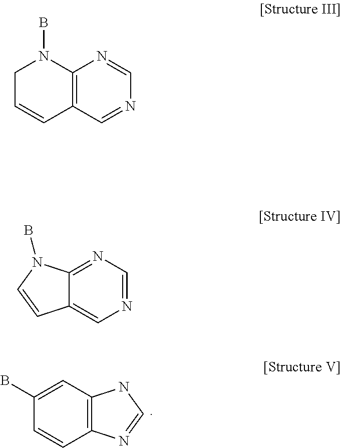

86. The method of claim 84, wherein B of Structure III or IV is a cyclopentane.

87. The method of claim 84, wherein B of Structure V comprises a pyrimidine.

88. The method of any one of the preceding claims, wherein the CDK4/6 inhibitor comprises the structure of 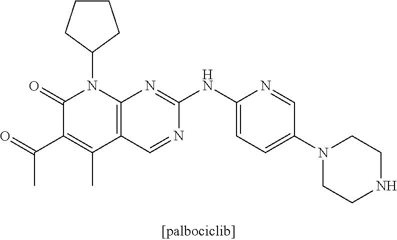 [SMILES: CC(=O)c1c(C)c2cnc(Nc3ccc(N4CCNCC4)cn3)nc2n(C2CCCC2)c1=O]
or a pharmaceutically acceptable salt thereof.

89. The method of any one of the preceding claims, wherein the CDK4/6 inhibitor comprises the structure of 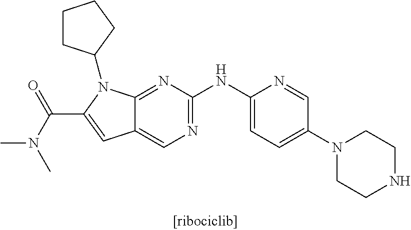 [SMILES: CN(C)C(=O)c1cc2cnc(Nc3ccc(N4CCNCC4)cn3)nc2n1C1CCCC1]
or a pharmaceutically acceptable salt thereof.

90. The method of any one of the preceding claims, wherein the CDK4/6 inhibitor comprises the structure of 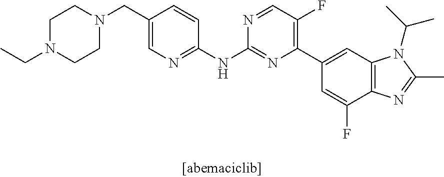 [SMILES: CCN1CCN(Cc2ccc(Nc3ncc(F)c(-c4cc(F)c5nc(C)n(C(C)C)c5c4)n3)nc2)CC1]
or a pharmaceutically acceptable salt thereof.

91. The method of any one of the preceding claims, wherein the CDK inhibitor is packaged separately from the RANK pathway inhibitor.

92. The method of any one of the preceding claims, further comprising administering to the subject a hormone therapy agent.

93. The method of claim 91, further comprising administering an aromatase inhibitor, an ER-targeted agent, rapamycin or a rapamycin analog, an anti-HER2 drug or a PI3K inhibitor.

94. The method of claim 92, wherein the aromatase inhibitor is letrozole, anastrozole, or exemestante.

95. The method of claim 92, wherein the rapamycin analog is everolimus, temsirolimus, ridaforolimus, zotarolimus, and 32-deoxo-rapamycin.

96. The method of claim 92, wherein the ER-targeted agent is fulvestrant or tamoxifen.

97. The method of claim 92, wherein the anti-HER2 drug is trastuzumab, pertuzumab, lapatinib, T-DM1, or neratinib.

98. The method of claim 92, wherein the PI3K inhibitor is taselisib, alpelisib or buparlisib.

99. A pharmaceutical composition of any one of the preceding claims for use in a method of any one of claims 39-98.


SEQUENCE LISTING (32 sequences)

SEQ ID NO: 1 - protein, 108 aa, Homo sapiens
  MISC_FEATURE: denosumab variable light chain
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 2 - protein, 122 aa, Homo sapiens
  MISC_FEATURE: denosumab variable heavy chain
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 3 - protein, 235 aa, Homo sapiens
  MISC_FEATURE: denosumab full length light chain
METPAQLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 467 aa, Homo sapiens
  MISC_FEATURE: denosumab full length heavy chain
MEFGLSWLFLVAILKGVQCEVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 12 aa, Homo sapiens
  MISC_FEATURE: Light Chain CDR 1
RASQSVRGRYLA

SEQ ID NO: 6 - protein, 7 aa, Homo sapiens
  MISC_FEATURE: Light Chain CDR 2
GASSRAT

SEQ ID NO: 7 - protein, 9 aa, Homo sapiens
  MISC_FEATURE: Light Chain CDR 3
QQYGSSPRT

SEQ ID NO: 8 - protein, 5 aa, Homo sapiens
  MISC_FEATURE: Heavy Chain CDR 1
SYAMS

SEQ ID NO: 9 - protein, 17 aa, Homo sapiens
  MISC_FEATURE: Heavy Chain CDR 2
GITGSGGSTYYADSVKG

SEQ ID NO: 10 - protein, 13 aa, Homo sapiens
  MISC_FEATURE: Heavy Chain CDR 3
DPGTTVIMSWFDP

SEQ ID NO: 11 - DNA, 2141 bp, Homo sapiens
ggccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccccgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacctgccagtgggagatgttagactcatggtgtgttacacaatggtttttaaattttgtaatgaattcctagaattaaaccagattggagcaattacgggttgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctataacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatttgtcaagagtagttatataattgttgaacaggtgtttttccacaagtgccgcaaattgtacctttttttttttttcaaaatagaaaagttattagtggtttatcagcaaaaaagtccaattttaatttagtaaatgttattttatactgtacaataaaaacattgcctttgaatgttaattttttggtacaaaaataaatttatatgaaaaaaaaaaaaaaaaaaaaaaaaaaaaaaaa

SEQ ID NO: 12 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 13 - protein, 215 aa, Artificial Sequence
  Synthetic Polypeptide
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 448 aa, Artificial Sequence
  Synthetic Polypeptide
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 15 - protein, 330 aa, Artificial Sequence
  Synthetic Polypeptide
  misc_feature (14)..(14): Xaa is Cys or Ser
  misc_feature (16)..(16): Xaa is Arg or Lys
  misc_feature (20)..(20): Xaa is Glu or Gly
  misc_feature (21)..(21): Xaa is Ser or Gly
  misc_feature (75)..(75): Xaa is Ser or Asn
  misc_feature (76)..(76): Xaa is Leu or Phe
  misc_feature (79)..(79): Xaa is Lys or Gln
  misc_feature (82)..(82): Xaa is Thr or Ile
  misc_feature (86)..(86): Xaa is Asp or Asn
  misc_feature (97)..(97): Xaa is Arg, Thr, or Lys
  misc_feature (100)..(100): Xaa is Ser, Arg, or Pro
  misc_feature (102)..(102): Xaa is Tyr, Cys, or Ser
  misc_feature (103)..(103): Xaa is Gly or Cys
  misc_feature (104)..(104): Xaa is absent or Asp
  misc_feature (105)..(105): Xaa is absent or Lys
  misc_feature (106)..(106): Xaa is absent or Thr
  misc_feature (107)..(107): Xaa is Pro, Val or His
  misc_feature (108)..(108): Xaa is Pro, Glu, or Thr
  misc_feature (111)..(111): Xaa is Ser or Pro
  misc_feature (116)..(116): Xaa is Glu or Pro
  misc_feature (117)..(117): Xaa is Phe, Val, or Leu
  misc_feature (118)..(118): Xaa is Leu or Ala
  misc_feature (120)..(120): Xaa is absent or Gly
  misc_feature (151)..(151): Xaa is Gln or His
  misc_feature (157)..(157): Xaa is Gln or Lys
  misc_feature (179)..(179): Xaa is Phe or Tyr
  misc_feature (183)..(183): Xaa is Tyr or Phe
  misc_feature (192)..(192): Xaa is Leu or Val
  misc_feature (210)..(210): Xaa is Gly or Ala
  misc_feature (213)..(213): Xaa is Ser or Ala
  misc_feature (214)..(214): Xaa is Ser or Pro
  misc_feature (222)..(222): Xaa is Ala or Thr
  misc_feature (238)..(238): Xaa is Gln or Arg
  misc_feature (239)..(239): Xaa is Glu or Asp
  misc_feature (241)..(241): Xaa is Met or Leu
  misc_feature (280)..(280): Xaa is Val or Met
  misc_feature (292)..(292): Xaa is Arg or Lys
  misc_feature (302)..(302): Xaa is Glu or Gln
  misc_feature (328)..(328): Xaa is Leu or Pro
ASTKGPSVFPLAPXSXSTSXXTAALGCLVKDYFPEPVTVSWNSGALTSGVHTFPAVLQSSGLYSLSSVVTVPSSXXGTXTYXCNVXHKPSNTKVDKXVEXKXXXXXXXCPXCPAPXXXGXPSVFLFPPKPKDTLMISRTPEVTCVVVDVSXEDPEVXFNWYVDGVEVHNAKTKPREEQXNSTXRVVSVLTVXHQDWLNGKEYKCKVSNKXLPXXIEKTISKXKGQPREPQVYTLPPSXXEXTKNQVSLTCLVKGFYPSDIAVEWESNGQPENNYKTTPPXLDSDGSFFLYSXLTVDKSRWQXGNVFSCSVMHEALHNHYTQKSLSLSXGK

SEQ ID NO: 16 - protein, 326 aa, Artificial Sequence
  Synthetic Polypeptide
  MISC_FEATURE: IgG2 Constant HC
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 330 aa, Artificial Sequence [sequence omitted]

SEQ ID NO: 18 - protein, 327 aa, Artificial Sequence
  Synthetic Polypeptide
  MISC_FEATURE: IgG4 constant HC
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - DNA, 324 bp, Artificial Sequence
  Synthetic Polynucleotide
  misc_feature: variable LC
gaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaa

SEQ ID NO: 20 - DNA, 366 bp, Artificial Sequence
  Synthetic Polynucleotide
  misc_feature: variable HC
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctca

SEQ ID NO: 21 - DNA, 705 bp, Artificial Sequence
  Synthetic Polynucleotide
  misc_feature: full length LC
atggaaaccccagcgcagcttctcttcctcctgctactctggctcccagataccaccggagaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 22 - DNA, 1401 bp, Artificial Sequence
  Synthetic Polynucleotide
  misc_feature: full length HC
atggagtttgggctgagctggctttttcttgtggctattttaaaaggtgtccagtgtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 23 - DNA, 645 bp, Artificial Sequence
  Synthetic Polynucleotide
  misc_feature: mature LC
gaaattgtgttgacgcagtctccaggcaccctgtctttgtctccaggggaaagagccaccctctcctgtagggccagtcagagtgttcgcggcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgttttactgtcagcagtatggtagttcacctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 24 - DNA, 1344 bp, Artificial Sequence
  Synthetic Polynucleotide
  misc_feature: mature HC
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattactgggagtggtggtagtacatactacgcagactccgtgaagggccggttcaccatctccagagacaattccaagaacacgctgtatctgcaaatgaacagcctgagagccgaggacacggccgtatattactgtgcgaaagatccagggactacggtgattatgagttggttcgacccctggggccagggaaccctggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 25 - protein, 20 aa, Artificial Sequence
  Synthetic Polypeptide
  MISC_FEATURE: signal peptide of full length LC of SEQ 3
METPAQLLFLLLLWLPDTTG

SEQ ID NO: 26 - protein, 19 aa, Artificial Sequence
  Synthetic Polypeptide
  MISC_FEATURE: signal peptide of full length HC of SEQ 4
MEFGLSWLFLVAILKGVQC

SEQ ID NO: 27 - protein, 10 aa, Artificial Sequence
  Synthetic Polypeptide
GFTFSSYAMS

SEQ ID NO: 28 - protein, 206 aa, Homo sapiens
TLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 30 - protein, 8148 aa, Homo sapiens
AGAGGCCGCTGAGGCCGCGGCGCCCGCCAGCCTGTCCCGCGCCATGGCCCCGCGCGCCCGGCGGCGCCGCCCGCTGTTCGCGCTGCTGCTGCTCTGCGCGCTGCTCGCCCGGCTGCAGGTGGCTTTGCAGATCGCTCCTCCATGTACCAGTGAGAAGCATTATGAGCATCTGGGACGGTGCTGTAACAAATGTGAACCAGGAAAGTACATGTCTTCTAAATGCACTACTACCTCTGACAGTGTATGTCTGCCCTGTGGCCCGGATGAATACTTGGATAGCTGGAATGAAGAAGATAAATGCTTGCTGCATAAAGTTTGTGATACAGGCAAGGCCCTGGTGGCCGTGGTCGCCGGCAACAGCACGACCCCCCGGCGCTGCGCGTGCACGGCTGGGTACCACTGGAGCCAGGACTGCGAGTGCTGCCGCCGCAACACCGAGTGCGCGCCGGGCCTGGGCGCCCAGCACCCGTTGCAGCTCAACAAGGACACAGTGTGCAAACCTTGCCTTGCAGGCTACTTCTCTGATGCCTTTTCCTCCACGGACAAATGCAGACCCTGGACCAACTGTACCTTCCTTGGAAAGAGAGTAGAACATCATGGGACAGAGAAATCCGATGCGGTTTGCAGTTCTTCTCTGCCAGCTAGAAAACCACCAAATGAACCCCATGTTTACTTGCCCGGTTTAATAATTCTGCTTCTCTTCGCGTCTGTGGCCCTGGTGGCTGCCATCATCTTTGGCGTTTGCTATAGGAAAAAAGGGAAAGCACTCACAGCTAATTTGTGGCACTGGATCAATGAGGCTTGTGGCCGCCTAAGTGGAGATAAGGAGTCCTCAGGTGACAGTTGTGTCAGTACACACACGGCAAACTTTGGTCAGCAGGGAGCATGTGAAGGTGTCTTACTGCTGACTCTGGAGGAGAAGACATTTCCAGAAGATATGTGCTACCCAGATCAAGGTGGTGTCTGTCAGGGCACATGTGTAGGAGGTGGTCCCTACGCACAAGGCGAAGATGCCAGGATGCTCTCATTGGTCAGCAAGACCGAGATAGAGGAAGACAGCTTCAGACAGATGCCCACAGAAGATGAATACATGGACAGGCCCTCCCAGCCCACAGACCAGTTACTGTTCCTCACTGAGCCTGGAAGCAAATCCACACCTCCTTTCTCTGAACCCCTGGAGGTGGGGGAGAATGACAGTTTAAGCCAGTGCTTCACGGGGACACAGAGCACAGTGGGTTCAGAAAGCTGCAACTGCACTGAGCCCCTGTGCAGGACTGATTGGACTCCCATGTCCTCTGAAAACTACTTGCAAAAAGAGGTGGACAGTGGCCATTGCCCGCACTGGGCAGCCAGCCCCAGCCCCAACTGGGCAGATGTCTGCACAGGCTGCCGGAACCCTCCTGGGGAGGACTGTGAACCCCTCGTGGGTTCCCCAAAACGTGGACCCTTGCCCCAGTGCGCCTATGGCATGGGCCTTCCCCCTGAAGAAGAAGCCAGCAGGACGGAGGCCAGAGACCAGCCCGAGGATGGGGCTGATGGGAGGCTCCCAAGCTCAGCGAGGGCAGGTGCCGGGTCTGGAAGCTCCCCTGGTGGCCAGTCCCCTGCATCTGGAAATGTGACTGGAAACAGTAACTCCACGTTCATCTCCAGCGGGCAGGTGATGAACTTCAAGGGCGACATCATCGTGGTCTACGTCAGCCAGACCTCGCAGGAGGGCGCGGCGGCGGCTGCGGAGCCCATGGGCCGCCCGGTGCAGGAGGAGACCCTGGCGCGCCGAGACTCCTTCGCGGGGAACGGCCCGCGCTTCCCGGACCCGTGCGGCGGCCCCGAGGGGCTGCGGGAGCCGGAGAAGGCCTCGAGGCCGGTGCAGGAGCAAGGCGGGGCCAAGGCTTGAGCGCCCCCCATGGCTGGGAGCCCGAAGCTCGGAGCCAGGGCTCGCGAGGGCAGCACCGCAGCCTCTGCCCCAGCCCCGGCCACCCAGGGATCGATCGGTACAGTCGAGGAAGACCACCCGGCATTCTCTGCCCACTTTGCCTTCCAGGAAATGGGCTTTTCAGGAAGTGAATTGATGAGGACTGTCCCCATGCCCACGGATGCTCAGCAGCCCGCCGCACTGGGGCAGATGTCTCCCCTGCCACTCCTCAAACTCGCAGCAGTAATTTGTGGCACTATGACAGCTATTTTTATGACTATCCTGTTCTGTGGGGGGGGGGGTCTGTTTTCCCCCCATATTTGTATTCCTTTTCATAACTTTTCTTGATATCTTTCCTCCCTCTTTTTTAATGTAAAGGTTTTCTCAAAAATTCTCCTAAAGGTGAGGGTCTCTTTCTTTTCTCTTTTCCTTTTTTTTTTCTTTTTTTGGCAACCTGGCTCTGGCCCAGGCTAGAGTGCAGTGGTGCGATTATAGCCCGGTGCAGCCTCTAACTCCTGGGCTCAAGCAATCCAAGTGATCCTCCCACCTCAACCTTCGGAGTAGCTGGGATCACAGCTGCAGGCCACGCCCAGCTTCCTCCCCCCGACTCCCCCCCCAGAGACACGGTCCCACCATGTTACCCAGCCTGGTCTCAAACTCCCCAGCTAAAGCAGTCCTCCAGCCTCGGCCTCCCAAAGTACTGGGATTACAGGCGTGAGCCCCCACGCTGGCCTGCTTTACGTATTTTCTTTTGTGCCCCTGCTCACAGTGTTTTAGAGATGGCTTTCCCAGTGTGTGTTCATTGTAAACACTTTTGGGAAAGGGCTAAACATGTGAGGCCTGGAGATAGTTGCTAAGTTGCTAGGAACATGTGGTGGGACTTTCATATTCTGAAAAATGTTCTATATTCTCATTTTTCTAAAAGAAAGAAAAAAGGAAACCCGATTTATTTCTCCTGAATCTTTTTAAGTTTGTGTCGTTCCTTAAGCAGAACTAAGCTCAGTATGTGACCTTACCCGCTAGGTGGTTAATTTATCCATGCTGGCAGAGGCACTCAGGTACTTGGTAAGCAAATTTCTAAAACTCCAAGTTGCTGCAGCTTGGCATTCTTCTTATTCTAGAGGTCTCTCTGGAAAAGATGGAGAAAATGAACAGGACATGGGGCTCCTGGAAAGAAAGGGCCCGGGAAGTTCAAGGAAGAATAAAGTTGAAATTTTAATTTGCATTTTTTTTGTCTAGATAAGAATAGCGTGAATAGATCCTCTTTTATTCGTAAATAATCGTGCATCTGTGGGTTAGCCTTGTAGAAGTGGAAAACATTCCATTTTCCAATGCATTTAAATGTAAAGCCAAATCTGCATGTTGTGAATTTAAGAAAACTTATTATCCTAAAGGTGCCTTTCTCTTGGCATCATCCCGCTTGTGAGAAGCCTAGAGGACGCTCCAGGTGGAAGGAAATCCCCTGGGTGGTTTTATCTTTTGTTACCCAGTGAGCACTGGTTCCCCGCAAATACTGGGGAAAAGCAAAAATACACAAGCAAGTTAAAATTAATTTTGCACATCTGGGAGGTTATAAAAGAAAGCACTAATAGTAGTCACTGCCCAGACTTTACTGGCCACAAATGCCCAGCTGAAGAGCATGACTGTGGATCACTGGTTTTTCCCTCCTGCTGGAAATGCTGGGGTGGTAGCGGTCGATTAGGATTTTCAGTGGAGAAGCACAGGACAGTTCTGTAATTTATGGGACTCCTTAGCCAACATAAAGAACTGCAGGAAATAACTGCACAGCCAGGAGGATCCGTTGGTGGGAATTTACCGTCATTCCTGCCCTTTTATTTAACATCATCCACAGAGAGATGTTATACAAATGGAGGAAACCATTATACCTTTTGATATGGAATATATTACAGAGTTACAGTTCACAAAGTAGAATGCTGAGCTGAAAACCCGAGTTTCTGCTGTGACTGTCATCTCACTGAGCACTTCGCTTTTCTTTGCTTTTATTTTTTCCTTCTATAAAAAGGCAATAATGATAATTTATAATAGTTTCCCTACCCAGAGATATTAGAAGTATGCTACAGTGAATGTTAAAGTACCTTGAGATCCTTAAATCAAAGGTGCTATATACATAAGTAAGACTCTACTTTCAGAAAAAGGTAATATTATTTCCTGCACTGATCCCTACTAATTCTATATTGATCCAAAGGCAACTCAATGCTAAAAAATGTATAGAAAATATAAGTCTGTGTCTGTGTACTGTAGAGATGTATGTGACAAGTGTAAACAAAATGAACTGAAGCAGTAATGAACAGTTATTAGGGGGAACATGATAAAGAGATTATATTAAGCTTATGTTTCACCATAAAATCCTTTTTATGGCTTACTAAAACCGAGCTCACTGTAAAATCATGATCCAACTTATTGCTAATCTTTATGATATGCTTATTCCTAATCTTTATGGTATGGTGTCAACCGTTCATTTGTATCTTATTGCTCATTCCCTGGACCACAGACTAGGGACAGAAAATACTTGCTTTAATAATATATATGCTGTTGATTTCACAAAAATTTATTAAAATACAGCCTGGGTACCCAGTGAAAGAGCTGAAATGGAAATGGAGTATCATGTTTCCTACTCACATTTTACTCAGCTGTCGGAAGGAAGAATGAATTTCTCAAGAGCAGTTTGAATGTGTTAGATCAGAACCTTCATGTGGTGGGGAAAATGGGATTATTTCAGTGTAACAAACCACCCAAAGACTTAGTGGCTTAAAATAGTCACCATTGGCCAGGCACAGCGGTTCATGCCTCCCAGTGCTTTGGGAGGCCAAGGCAGGAGGATCACTTGAGGACAGTAGTTAGAGATCAAACTGGGCAATGGAGTGAGACCTCGTCTCTACAAAAAATACAAAACTTATCTGGGCACGGTGGTGTGCACCTGCAGTCCTGGCTACTCAGGAGGCTGAGGCAGGAGGATGCTTGAGTCCAGGAGTTCAAGGTTACTGTGAGCTTTGATTGGGTCAGTGCACTCCAGTGTAAAAAAACAAAAATAAAAACCACCACTTATTTAGCTCCCAATTCTATGGGTTGGATCAGCTTGGGTGGTTCTTCAGGCCTGGTCCCTCCTGGCCTGGTTTGCTTACCCATTTACAATGACCCGGCTAGTTGGCCGGGCCAGGCTGTGTGCTCTCCACCTGTCTCCCATCCTTCCAGCCACCTAGCTGGGCATGCTCTCATGGTGGTGTCGAGCCTGAGAACAAGCAGAAACCTGCCAGGCTTCTTGAGGCCTAGGCTCTAACTGGCATGCTGTCACTTCTGCCACATTCTGTGGGCCAAAGCAGGTCACAAAGGCAGCCCAGATCCAAGGGATGGGGGAGCAGCATATCCCCCCATGGGAAGAGTTGCGGAATGAGAGGGTAGGGGGTGGACACACAGAGGGGAAGATGGCCCCCTTGCCATCTGTCCACCACAGGGCACTGCGTGGAGTAAAATGCTGCAGAGAAGAGTGCATTCTGGGAAAAAAAGAGGAACATATCCCAGCCCCTAACCAGTGAGTAGCTCCTTCTCTGGTGATTGAATTCCCTTAATGCACAGCACTATAGATTCCCACAATGAAAGGGACACCCACGTTCATATTTAAGTGCTGTGTCAAAATTCTTTCCAGAGCCAAATATAGGTGATTTGGGAATTGAGCCTACTGTACTGCTTCCACATTCCTTTGGCACAGAAAACTGAAAAGTCACAACTTCTAAAATCAAGAAAACTGGTGAGATACCTTGGGGTAGAGAAACCTGCATATAAACAGATAAAAGATCGAGTGTTCACCCTTCACCTCAGCTCAACAAACAAACTCTTGTTACGATTATACAAGGGTGAGCTTGAGCAAAGGTGCTGTCTTTGGTGGAAGAAACGTAGTTTTGAGTTGTTTGCTCTTTGACAGTCTCTGATGGCATGTGAGTCTGGTGCATTGCTGTGGGCTGCGCTGGGCACAAGTTGCATAACCCGCTTTTGGAAGAGGAGACAAAGCATTCAAAGCCCTAAACACTCTGTTGGTGAATCATCATGTTTATTGAGTGTGCTTTTAGTGGCAGGGCCCTGAGCACCTGGGAAAGGAGATGAGGAGCGCTGGAGGGTTCGGAGCAGAGAAGACGAGACAATGCTCTGCTCCAAGCGCCAGAGGAGGCTTCTGGGAAGGGGCTGTCTGTGGAATTTCTGACCCCCAGAGCCCTGCTGGTGAAGTCTTGCAACCACTAAAGCTTTGCCAGAGAAGAGGAGGGACCTGACACTGGGCAGTGGCGGTGCCAGCAGTGAGCACAGGCGGGGTCTAGGTGGGATCACTGAAGCCAAGCCAAGTGGTGCAGGGGGAGCCGCCCTGGACCAACACCCCCATGGCTGCCCCACTGCGGAACTGCCTACTGCGGCAAAACAATCCTATGTCCCCCAAGGTTTCAACAAAGAGGAGGACCGAATAAATGTCCCAGTGGGCAGGCACACCCACAAGACTTGCTGCTCCTCCTGACCCACCCGCATGGCTCTGCACAGCTGTGAGTCCAGTTTTCTCTCTTCCTACCTCCCTCTTACCCAGTTATCCTGACCCCAGGGTGAGGTTGAAACTCATGCTAGGCTAACATCTCTTTCCTTCATCCCATTCATAAAACTGGCATACAGGAATGTTTTAAAAAAATCTTTAAGATTACCAGGAGTATAAAAGTTAAAATGACAAAATTACAGGCCTGTTACCAGATCTTCGTACTAACCAAGGAGCTGGTGACACTACCCTCTTCCCTGTGTGATAGAAAATGCAGACGTTTTGCAGGCAGACCTGGTTCATGACTCAGCGCTGTGACTTGTGGACTTATCTTGTGCAGCTACATCACTTCTTAGCCTTGTTTTCAACATGCGTAAGCAGAAGGTACTAATATTAGTCTTTAGGGCTGGTTTGAGGATGACGTGAGATGAGAGACAACCAAGCACTCACTGGCAGTTCCTAGAAGATTCCAGTAGTAGAGATCTGACTGTCCTACACTACTTTCCCTCCCTGTCTCTGACACTTGTTCATTCCTGGGCTCAGGAGCCATCTCAGTCATCATGTCGGACCACGTAACTATAACCAAGCTCTGCTGCCCACCTGGGACACCTTCCCACTCTTTGCAACTAGGTCGGAGGGACGCACCTTCTCCACAGTTGACAGGACTGTTATGGTTGACCCAAAAGTTGTCATCTTCTGACATGACACACTGAGGGAAGTAGACTTACCCAATATAACTTCACATTATACTGTCTTAAAAGACTATGATTAAAATGGGCATCAGTGTAGGCAACATGTAGGGCGCAAAGAATAGCCAGATGTGAAGGTTGAAAGGCTATGTGGGCTAGTTTGTCATGGTATTCCTGGTGCCTCTCCCCGTTTGGGAAGAATAACACATTGACCACCGGCTCAGAGGAGGTGGCATTATAGGTCCAGGCCCCTTCAGGAGGGCACTGGAGTCAATGAAAGCTTTTGCTCTCACCCAGGCTCTTGGTTTCTCACGGGCAGGGGTAGCCCAGGAAACAAAGGCTGTATAGACTTTAAGTCGTGAGAAAAACGACATTCTTATGGTGAGGCTCTTAGAGACTGTCTCCATTTAAGCAGCAGCAGTAAAACATCTTACCCTAAAATGAAGAAGCAAATCCATTTTTATTGGCTTTTATAGCCTCTCTCTTTCAATGACAAAGAAACCATTAAAATGAATGTTTAAAGTTCTTAGAAGAGCTTTCTCTAAATATTTACTCTGTCAGTGGAAAAATAAACATTTTTTACATCGGGTCTATTGAGTTTGTTATATACAGTAAAATTCACCCTTTTTAGATATATAGTTTTATGAGTTTTAACAAAAATACATAGTTGTGTAACCACCACCATCACAATCAAGATATAGAATATTTCAACATCCCAAAGTTCCCATTTCCCTTTTCAGTCAATCCCTTCCCCACTACCCCTAGCCCCTGGCAACCATTATCTGTTCTCTCTTCCCATAGTTTCACCTTTTCCAATTTGTCGTGTAGAGTTACACAGCTCATAGCCATTTGAGTCTGGCTTCTTTCCACATAATGTCCTTGAGATCCATCGGTGGGTACACCAGTAGTTCCATTTTTTTTCTGAGTAGTAGTCCATTGTATGGATGTACTGCGGTTTATTCACCAACATATGGGTTCTTTCCTGTTTGGGGAGATTACAAATAAAGCTACTATAAAAA

SEQ ID NO: 31 - protein, 317 aa, Homo sapiens; identical to SEQ ID NO: 12

SEQ ID NO: 32 - DNA, 2201 bp, Homo sapiens
gcccgctcgcccgcgcgccccaggacccaaagccgggctccaagtcggcgccccacgtcgaggctccgccgcagcctccggagttggccgcagacaagaaggggagggagcgggagagggaggagagctccgaagcgagagggccgagcgccatgcgccgcgccagcagagactacaccaagtacctgcgtggctcggaggagatgggcggcggccccggagccccgcacgagggccccctgcacgccccgccgccgcctgcgccgcaccagccccctgccgcctcccgctccatgttcgtggccctcctggggctggggctgggccaggttgtctgcagcgtcgccctgttcttctatttcagagcgcagatggatcctaatagaatatcagaagatggcactcactgcatttatagaattttgagactccatgaaaatgcagattttcaagacacaactctggagagtcaagatacaaaattaatacctgattcatgtaggagaattaaacaggcctttcaaggagctgtgcaaaaggaattacaacatatcgttggatcacagcacatcagagcagagaaagcgatggtggatggctcatggttagatctggccaagaggagcaagcttgaagctcagccttttgctcatctcactattaatgccaccgacatcccatctggttcccataaagtgagtctgtcctcttggtaccatgatcggggttgggccaagatctccaacatgacttttagcaatggaaaactaatagttaatcaggatggcttttattacctgtatgccaacatttgctttcgacatcatgaaacttcaggagacctagctacagagtatcttcaactaatggtgtacgtcactaaaaccagcatcaaaatcccaagttctcataccctgatgaaaggaggaagcaccaagtattggtcagggaattctgaattccatttttattccataaacgttggtggattttttaagttacggtctggagaggaaatcagcatcgaggtctccaacccctccttactggatccggatcaggatgcaacatactttggggcttttaaagttcgagatatagattgagccccagtttttggagtgttatgtatttcctggatgtttggaaacattttttaaaacaagccaagaaagatgtatataggtgtgtgagactactaagaggcatggccccaacggtacacgactcagtatccatgctcttgaccttgtagagaacacgcgtatttacagccagtgggagatgttagactcatggtgtgttacacaatggtttttaaattttgtaatgaattcctagaattaaaccagattggagcaattacggggtgaccttatgagaaactgcatgtgggctatgggaggggttggtccctggtcatgtgccccttcgcagctgaagtggagagggtgtcatctagcgcaattgaaggatcatctgaaggggcaaattcttttgaattgttacatcatgctggaacctgcaaaaaatactttttctaatgaggagagaaaatatatgtatttttatataatatctaaagttatatttcagatgtaatgttttctttgcaaagtattgtaaattatatttgtgctatagtatttgattcaaaatatttaaaaatgtcttgctgttgacatatttaatgttttaaatgtacagacatatttaactggtgcactttgtaaattccctggggaaaacttgcagctaaggaggggaaaaaaatgttgtttcctaatatcaaatgcagtatatttcttcgttctttttaagttaatagattttttcagacttgtcaagcctgtgcaaaaaaattaaaatggatgccttgaataataagcaggatgttggccaccaggtgcctttcaaatttagaaactaattgactttagaaagctgacattgccaaaaaggatacataatgggccactgaaatctgtcaagagtagttatataattgttgaacaggtgtttttccacaagtgccgcaaattgtaccttttttgttttttcaaaatagaaaagttattagtggtttatcagcaaaaaagtccaattttaatttagtaaatgttatcttatactgtacaataaaaacattgcctttgaatgttaattttttggtacaaaaataaatttatatgaaaacctgc